(19) United States
(12) Patent Application Publication
(10) Pub. No.: US 2015/0285789 A1
(43) Pub. Date: Oct. 8, 2015

(54) ASSAYS, SYSTEMS, AND METHODS FOR OBTAINING PERSONALIZED ANABOLIC PROFILES

(71) Applicant: BOSTON MEDICAL CENTER CORPORATION, Boston, MA (US)

(72) Inventor: Monty Montano, Brookline, MA (US)

(21) Appl. No.: 14/423,013

(22) PCT Filed: Aug. 20, 2013
(86) PCT No.: PCT/US2013/055749

§ 371 Date: Feb. 20, 2015

Related U.S. Application Data
(60) Provisional application No. 61/684,959, filed on Aug. 20, 2012.

(51) Int. Cl. G01N 33/50; A61K 31/436; G06F 19/00; C12N 5/077

(52) U.S. Cl. CPC G01N 33/5061; C12N 5/0658; A61K 31/436; G06F 19/345; G06F 19/3487; C12N 2501/155

(57) ABSTRACT

Disclosed herein are novel assays, systems and kits for generating a personalized or stratified diagnostic report, e.g., to facilitate selection of an appropriate treatment of a musculoskeletal disease or disorder in a subject. The assays and systems are based on detecting and/or measuring in vitro anabolic responses of musculoskeletal cells or precursor cells thereof to a panel of test compositions (e.g., test compositions each comprising at least one agent selected to maintain and/or increase muscle and/or bone growth), and ranking the ability of each test composition to stimulate muscle and bone growth. The resultant ranks of the test compositions can then be used to identify, select, and/or optimize a treatment regimen for the subject who is determined to have, or have a risk, a musculoskeletal disease or disorder. Methods for treating and/or preventing a musculoskeletal disease or disorder are also provided herein.

### CROSS REFERENCE TO RELATED APPLICATIONS

This application claims the benefit under 35 U.S.C. §119(e) of U.S. Provisional Application No. 61/684,959 filed Aug. 20, 2012, the content of which is incorporated herein by reference in its entirety.

### TECHNICAL FIELD

The inventions provided herein generally relate to assays, systems, and kits for generating a personalized anabolic profile, which can be used in diagnostics, therapeutic and/or nutritional decision support. The inventions also relate to methods for selecting a treatment regimen for a subject determined to have a musculoskeletal disease or disorder.

### BACKGROUND OF THE DISCLOSURE

Loss of muscle mass is increasingly common in the aging population and is present in a variety of debilitating muscle wasting associated disease. It can also be associated with chemotherapy, and weight loss. Muscle wasting and bone loss can reduce quality of life, increase risk for mortality and pose a substantial burden on the healthcare system. For example, in industrialized countries (e.g., North America, Europe, Japan), the overall prevalence of cachexia (due to any disease) is growing and currently about 1%, i.e., about nine million patients. Musculoskeletal wasting includes both muscle decline and/or bone loss. Muscle decline indications include, for example, HIV associated wasting, cachexia and muscular dystrophy. Bone loss indications include, for example, HIV associated bone loss, osteopenia and osteoporosis. For HIV/AIDS, the United States has a 0.6% rate of prevalence, with approximately 1.2 million HIV infected individuals. For cachexia, in the United States, cancer cachexia affects more than 1.3 million people (˜30% of all individuals with cachexia) (C.C.M.H. Group, 2012). The total number of individuals inflicted with cachexia is ˜4 million people in the US. For muscular dystrophy, about 500-600 male newborns are usually diagnosed with muscular dystrophy each year in the US. Osteoporosis is a major health risk for about 28 million Americans. In the United States, about 10 million individuals have osteoporosis and about 18 million more have low bone mass (NIAMS, 2012).

Current diagnostic and treatment procedures do not distinguish between different types and/or causes of musculoskeletal decline and do not provide guidance for anabolic therapy. For example, existing diagnostics include measurement of creatine kinase (CK) and phosphocreatine (PCr) in muscle biopsies as a measure of muscle decline. Urine urea is used to infer rapid loss of muscle. Electromyography (EMG) is used to measure neuromuscular function using a surface electrode. Standardized reference values for body composition (e.g., National Health and Nutrition Examination Survey (NHANES)) are used in assessing loss of skeletal muscle. Body composition assessments through the use of computed tomography (CT) image analysis or dual-energy X-ray absorptiometry (DXA) can be used to quantify loss of skeletal muscle or bone, respectively. While there is a desire to use less invasive procedures such as CT or DXA, these less-invasive procedures (e.g., CT or DXA) do not provide information about muscle function or personalized anabolic therapeutic options.

The current standard of care for muscle wasting is to provide nutritional supplementation and anabolic supplements such as testosterone, analogs of testosterone (e.g., DHT) growth hormone or analogs of growth hormone. The current standard of care for bone loss is vitamin D supplementation, the use of bisphosphonates and/or bone resorption antagonists. The relative anabolic efficacy of these compounds—on a patient specific level—is unknown and not currently part of routine care. Human genetic variation and life history influence, often unpredictably, the response to therapeutic intervention. For example, HIV progression and response to drugs can be influenced by genetic polymorphisms (A. Telenti et al., 2008 Annu Rev. Pharmacol. Toxicol. 48: 227). Cancer cachexia can vary in severity and response (Tan B. H. et al. 2011 J. Genet 90: 165). Muscular dystrophy can vary in progression and responsiveness (Pegoraro E. et al., 2010 Neurology 76: 219). Additionally, although anabolic compounds promote mass gains in muscle and/or bone, they do so with varying efficacy that depends on many factors, including age (See, e.g., Banerjee C. et al., 2011 Immun. Aging 8:5). These diagnostic-treatment deficiencies limit treatment options to help guide patient and disease specific treatment.

In 2010 over 18 million people in the United States were estimated to suffer from age-associated decline in muscle mass and function (e.g., sarcopenia). Although anabolic supplementation has been used in treating sarcopenia, HIV muscle wasting and cachexia, anabolic responses vary in individuals and there are consistently 15-25% of individuals that do not benefit and are in effect non-responders. See, e.g., Montano et al. Journal of Clinical Endocrinology & Metabolism, 2007, 92 (7): 2793-2802; Sardar et al. HIV Clin Trials, 2010 July-August, 11(4): 220-229; and Houtman et al., Anal Chim Acta, 2009, 637(1-2): 247-258. The financial burden on healthcare is predicted to increase as the US population ages, in part due to loss in muscle mass and the associated increased risk for functional decline. This anticipated burden poses an immediate challenge to identify effective diagnostic tools that better match patients with treatment options. Accordingly, there is a need for development of an assay or a diagnostic test that can be used to provide a more personalized guidance on anabolic therapy, thus improving the treatment outcome and/or response rate.

### SUMMARY

Current diagnostic and treatment procedures do not distinguish between different types and/or causes of musculoskeletal decline and do not provide guidance for anabolic therapy specific to an individual. For example, there are no predicate products (e.g., FDA approved medical device) or commercially-available kits, which can be used to guide a personalized or stratified anabolic therapy. Conventional diagnostics typically measure muscle loss, and do not indicate type of deficit or responsiveness to anabolic alternatives. Accordingly, there is a need for development of assays, methods and/or kits that can provide a personalized or stratified, and scalable, anabolic guide to maintain muscle health and/or optimize treatment of musculoskeletal wasting, thus improving health outcomes and reducing healthcare costs associated with musculoskeletal decline in muscle and/or bone disease as well as in aging.

The inventions provided herein generally relate to assays, methods, systems, and kits, e.g., for profiling anabolic responses of a subject or a population subgroup to a panel of anabolic agents or compositions selected to maintain and/or increase muscle and/or bone growth. The assays, methods, systems, and kits described herein, in part, rely on ranking relative efficacies of the anabolic agents or compositions in stimulating muscle and/or bone growth of subject-specific cells (i.e., patient-specific cells) or a panel of cells representing different population subgroups, thereby generating a personalized or stratified diagnostic report. In some embodiments, the panel of cells representing different population subgroups can be stratified based one or a plurality of (e.g., at least two or more) feature(s) (e.g., phenotypic feature(s) including, but not limited to, age, gender, body mass index (BMI), condition, and/or ethnicity) of the population subgroups. Thus, an anabolic agent or composition can be selected, recommended and/or optionally administered to a subject or a patient in need thereof based on the personalized or stratified diagnostic analysis. In some embodiments, an anabolic agent or composition can be selected, recommended and/or optionally administered to a subject or patient based on a subject-specific or patient-specific anabolic profile generated using his/or own biopsy and/or blood sample. In alternative embodiments, an anabolic agent or composition can be selected, recommended and/or optionally administered to a subject or patient based on a stratified anabolic profile generated using tissue specimens of a matching population subgroup as the subject, wherein the subject is matched or associated to an appropriate population subgroup based on one or a plurality of pre-determined feature(s) such as phenotypic feature(s). By way of example only, for a 30-year old diabetic Caucasian woman who is in need of muscle augmentation and/or mitigation of muscle or bone loss, an optimal anabolic agent or composition can be selected for her based on a personalized anabolic profiling (which requires her own biopsy or blood sample) and/or a stratified anabolic profiling of a population subgroup of about 30-year old (e.g., 25-35-year old) diabetic Caucasian women.

Unlike the existing diagnostic methods such as CT and MRI, or blood biomarkers such as creatine phosphor-kinase (CPK), the generated functional anabolic profiles can provide information about muscle and/or bone function in response to a variety of anabolic agents or compositions, which can in turn be used to make diagnostic, and/or therapeutic or prophylactic decisions. For example, in some embodiments, the generated functional anabolic profiles can be used to diagnose an anabolic deficiency, and/or a defect in and/or an imbalance between anabolic growth pathway(s) in a subject. In some embodiments, the generated functional anabolic profiles can be used to identify and/or optimize a therapeutic or nutritional option for treatment of a muscle wasting-associated disease or disorder. In other embodiments, the generated functional anabolic profiles can be used to identity and/or optimize a prophylactic option to prevent or mitigate muscle loss and to optimize and maintain muscle heath. Accordingly, methods for diagnosing, treating and/or preventing muscle wasting or a musculoskeletal disease or disorder in a subject are also provided herein.

In one aspect, provided herein relates to cell-based assays, e.g., which can be carried out to obtain personalized or stratified anabolic profiles. The assay comprises: (a) contacting a population of musculoskeletal cells or precursor cells thereof with a plurality of test compositions each comprising at least one agent selected to increase and/or maintain muscle and/or bone growth, to profile anabolic responses of the cells to the test compositions; (b) subjecting the musculoskeletal cells or precursor cells thereof to at least one analysis, including, e.g., at least two analyses, to quantify muscle growth and/or bone growth of the musculoskeletal cells or precursor cells in response to the test compositions; and (c) ranking anabolic efficacy of the plurality of the test compositions based on the quantified muscle growth and/or bone growth, thereby providing anabolic profiles for muscle and/or bone growth of the assayed cells.

In some embodiments, the musculoskeletal cells or precursor cells thereof for use in the assay described herein can be obtained or derived from a muscle biopsy or a blood sample of a subject or patient seeking for an anabolic treatment or supplement. In one embodiment, muscle stem cells derived from a subject's biopsy or blood sample can be subjected to the assay described herein. Thus, the generated anabolic profile for muscle growth and/or bone growth can be personalized to the specific subject or patient.

In alternative embodiments, the musculoskeletal cells or precursor cells thereof for use in the assay described herein can be obtained or derived from cells or tissue specimens representing one or more different population subgroups. The cells or tissue specimens representing one or more different population subgroups can be obtained from a cell or tissue depository. In these embodiments, a stratified anabolic profile of a population subgroup that shares at least one or more features with a subject seeking for an anabolic treatment and/or supplement can be used to determine an optimal treatment and/or supplement for the subject. Examples of a feature can be a phenotypic feature for population stratification including, but not limited to, age groups, gender, ethnicity, body types, body mass index (BMI), blood types, activity levels, a condition such as chronic or acute diseases and/or psychophysiological disorders, genetic polymorphisms, diet, drug resistance, treatment regime such as chemotherapy, drastic/abnormal weight loss, geographical location, and any combinations thereof. In some embodiments, the stratification can be performed based on age and gender.

In some embodiments, the musculoskeletal cells or precursor cells thereof for use in the assay described herein can be obtained or derived from a biological sample (e.g., but not limited to, a muscle biopsy and/or blood sample) of subjects or individuals who are determined to suffer from or have a risk for muscle loss and/or bone loss (e.g., a musculoskeletal disease or disorder). Examples of subjects or individuals who are at risk for a musculoskeletal disease or disorder include, but are not limited to, athletes, aging individuals, individuals having a chronic disease or disorder (e.g., but not limited to, cancer, chronic obstructive pulmonary disease (COPD), chronic kidney disease (CKD), chronic liver failure (CLF), and chronic infections), individuals suffering from malnutrition, or any combinations thereof. Examples of a musculoskeletal disease or disorder include, but are not limited to, muscle loss, muscle wasting, muscle wasting associated with HIV infection, muscle wasting in cancer survivors, cachexia, muscular dystrophy, osteopenia, osteoporosis, sarcopenia, an age-related musculoskeletal disease or disorder, or a musculoskeletal disease or disorder associated with anabolic resistance, a musculoskeletal disease or disorder associated with excessive weight loss, or any combinations thereof.

In some embodiments, the musculoskeletal cells or precursor cells thereof for use in the assay described herein can be obtained or derived from a biological sample of subjects or individuals who have previously shown non-responsiveness or resistance to at least one or more anabolic agents.

In some embodiments, the musculoskeletal cells or precursor cells thereof for use in the assay described herein can be obtained or derived from a biological sample of subjects or individuals who are seeking to maintain and/or enhance muscle and/or bone health.

In order to determine specific anabolic responses (e.g., muscle growth or bone growth) of the musculoskeletal cells or precursor cells thereof to one or more test compositions, the cells are cultured in appropriate conditions optimized for each specific anabolic response (e.g., muscle growth or bone growth). For example, to determine the muscle growth-response of the musculoskeletal cells or precursor cells thereof to a plurality of test compositions, it can be desirable to culture and/or maintain the cells in a muscle cell-specific condition (e.g., a condition optimal to muscle cell differentiation) during the contact with the test compositions. In some embodiments, the muscle cell-specific condition can include culturing in a substrate material with a defined stiffness optimal to muscle cell differentiation. For example, the muscle cell-specific condition can include culturing in a substrate material with a defined stiffness of about 5 kPa to about 50 kPa, or about 10 kPa to about 20 kPa.

Similarly, to determine the bone growth-response of subject-specific cells to a plurality of test compositions, it can be desirable to culture and/or maintain the musculoskeletal cells or precursor cells thereof in a bone cell-specific condition (e.g., a condition optimal to bone cell differentiation) during the contact with the test compositions. In some embodiments, the bone cell-specific condition can include culturing in a substrate material with a defined stiffness optimal to bone differentiation. For example, the bone cell-specific condition can include culturing in a substrate material with a defined stiffness of about 10 kPa to about 150 kPa, or about 20 kPa to about 100 kPa. While not necessary, in some embodiments, the bone cell-specific condition can further include culturing in the presence of a bone formation-inducing agent. Examples of a bone formation-inducing agent can include, but are not limited to, bone morphogenic factor (BMP) (e.g., BMP-1, BMP-2, BMP-3, BMP-4, BMP-5 and BMP-6), transforming growth factor (TGF), insulin-like growth factor (IGF), basic fibroblast growth factor (bFBF), osteogenic protein (OP) (e.g., OP-1, OP-2 and OP-3), osteogenic factors, osteoconductive factors, osteoinductive factors, and any combinations thereof. In one embodiment, the bone formation-inducing agent can include bone morphogenetic protein-2 (BMP-2).

Quantitation of anabolic responses (e.g., muscle growth and/or bone growth) can be performed by any methods known in the art. For example, muscle growth of a subset of the musculoskeletal or precursor cells thereof in response to a test composition can be quantified based on a distribution of the number of nuclei per cell. The number of nuclei per cell can be determined, for example, by cell imaging. Thus, in one embodiment, an increase in muscle growth of the musculoskeletal or precursor cells thereof induced by a test composition can be quantified by an increase in the number of multi-nucleated cells formed by fusion of the musculoskeletal cells or precursor cells thereof (e.g., mononucleated muscle cells), as compared to a condition without the test composition.

Bone growth can be characterized by differentiation of muscle cells or bone precursor cells thereof to bone cells. For example, in some embodiments, an increase in bone growth of the musculoskeletal or precursor cells thereof induced by a test composition can be characterized by an increase in the number of bone cells differentiated from the musculoskeletal cells or precursor cells thereof (e.g., muscle cells or bone precursor cells thereof), as compared to a condition without the test composition. Any art-recognized methods can be used to characterize bone differentiation. For example, in one embodiment, the bone cells can be identified by detecting expression of a bone marker. An exemplary bone marker includes, but not limited to, alkaline phosphatase (ALP), type I collagen propetides, osetocalcin, and any combinations thereof.

The test compositions used in the assay described herein can each independently comprise one or more agents selected to increase and/or maintain muscle and/or bone growth. In some embodiments, at least some of the test compositions can comprise two or more agents selected to increase and/or maintain muscle and/or bone growth. The agent(s) included in the test compositions can include a therapeutic agent that has already been indicated for anabolic treatment (e.g., FDA-approved anabolic drugs or over-the-counter anabolic drugs), off-label FDA-approved drugs or over-the-counter drugs, an anabolic supplement, a candidate agent to be assessed for its anabolic efficacy, or any combinations thereof. Thus, in some embodiments, the assays described herein can be used to identify a novel anabolic compound or a novel combination of anabolic compounds suitable for a subject's or a population subgroup's musculoskeletal condition. In other embodiments, the assays described herein can be used to select or optimize a treatment regimen for a subject with a musculoskeletal condition, e.g., selecting a specific anabolic agent or combination therapy that stimulate muscle and bone growth in the subject, and/or optimizing the dosage and/or administration schedule of the selected anabolic agent(s) for a personalized treatment. Accordingly, in some embodiments, the assay can further comprise identifying or selecting at least one of the test compositions for administration to the subject, wherein the at least one of the test compositions is selected based on the rankings of their anabolic efficacies in the assay. The selected test composition for administration to the subject can provide a therapeutic effect for treatment of a musculoskeletal disease or disorder in a subject, or a prophylactic effect for optimizing and maintaining muscle health in a subject.

In some embodiments, the agent(s) included in the test compositions can include a molecule that is involved in an anabolic growth pathway. Examples of an anabolic growth pathway can include, but are not limited to, an amino acid pathway, an androgen receptor (AR): testosterone (T) pathway, a Wnt pathway, a calcium pathway, an IGF pathway, an insulin pathway, a follistatin pathway, a growth hormone pathway, an adhesion G-protein coupled receptor (GPCR) pathway, a myostatin pathway, and a FGF pathway. By way of example only, if the musculoskeletal or precursor cells thereof corresponding to a subject or a population subgroup do not respond to a subset of test compositions associated with a specific anabolic growth pathway, the subject can be diagnosed for having a defect in the specific anabolic pathway, or a disease or disorder associated with the defective anabolic pathway. Accordingly, in some embodiments, the assay can further comprise identifying or diagnosing an anabolic deficiency or a defect in or an imbalance among anabolic pathways in a subject or population subgroup based on the anabolic responses of the respective cells to the test compositions.

In some embodiments, the assays described herein can be employed as part of a clinical decision support to optimize or select a treatment regimen for a subject determined to have, or have a risk for, a musculoskeletal disease or disorder. Accordingly, another aspect provided herein relates to a method of optimizing or selecting a treatment regimen for a subject determined to have, or have a risk for, a musculoskeletal disease or disorder. The method comprises subjecting the musculoskeletal cells or precursor cells thereof obtained or derived from a subject determined to have, or have a risk for, a musculoskeletal disease or disorder, or a group of individuals sharing a similar background and symptoms as the subject, to one or more embodiments of the assay described herein. The test compositions can be ranked based on its efficacy to stimulate muscle and/or bone growth as determined in the assay. If some of the test compositions show an anabolic efficacy above a pre-determined threshold (e.g., anabolic response of the musculoskeletal or precursor cells thereof in the absence of the test composition), at least one of those test compositions can be selected, based on their ranking in the assay described herein, for administration to the subject. If none of the test compositions demonstrates an anabolic efficacy above the pre-determined threshold, none of the test compositions is selected or recommended for the treatment.

Methods of treating a subject determined to have, or have a risk for, a musculoskeletal disease or disorder are also provided herein. In one aspect, the method comprises subjecting the musculoskeletal cells or precursor cells thereof obtained or derived from a subject determined to have, or have a risk for, a musculoskeletal disease or disorder, or a group of individuals sharing a similar background and symptoms as the subject, to one or more embodiments of the assay described herein. If any of the test compositions demonstrates an anabolic efficacy above a certain threshold (e.g., anabolic response of the subject-specific cells in the absence of the test composition), at least one of those test compositions can be selected based on its ranking in the assay to treat the subject. In such embodiments, the method can further comprise prescribing or administering an effective amount of the selected test composition to the subject. On the other hand, if none of the test compositions demonstrates an anabolic efficacy above the threshold, none of the test compositions is selected or recommended for the treatment.

In another aspect, a method of treating a subject determined to have, or have a risk for, a musculoskeletal disease or disorder comprises administering to a subject determined to have, or have a risk for, a musculoskeletal disease or disorder, an effective amount of a test composition selected based on its ranking in the assay described herein. In some embodiments, the method can further comprise performing the assay with the musculoskeletal cells or precursor cells thereof obtained or derived from the subject, or a group of individuals sharing a similar background and symptoms as the subject.

In some embodiments, the assays described herein can be employed as part of a preventive care for individuals seeking to mitigate or prevent loss in muscle and bone, e.g., on a routine basis to extend health-span. Accordingly, methods of preventing a musculoskeletal disease or disorder in a subject, or maintaining or increasing muscle and/or bone mass in a subject are also provided herein. In one aspect, the method comprises subjecting the musculoskeletal cells or precursor cells thereof obtained or derived from a subject determined to have a muscle and/or bone loss, or experience a symptom associated with an onset of a muscle and/or bone loss, or from a group of individuals sharing a similar background and symptoms as the subject, to one or more embodiments of the assay described herein. If any of the test compositions indicates a reduction or delay in the onset of muscle and/or bone loss, at least one of those test compositions can be selected based on its ranking in the assay as a preventative supplement. In such embodiments, the method can further comprise prescribing or administering an effective amount of the selected test composition to the subject. On the other hand, if none of the test compositions indicates a reduction or delay in the onset of muscle and bone loss, none of the test compositions is selected or recommended as a preventative supplement.

In another aspect, the method of preventing a musculoskeletal disease or disorder in a subject, or maintaining or increasing muscle and/or bone mass in a subject comprises administering to a subject determined to have a loss in muscle and/or bone, or experience a symptom associated with an onset of a loss in muscle and/or bone, an effective amount of a test composition selected based on its ranking in the assay described herein. In some embodiments, the method can further comprise performing the assay with the musculoskeletal cells or precursor cells thereof obtained or derived from the subject, or a group of individuals sharing a similar background and symptoms as the subject.

In some embodiments of the methods of various aspects described herein, the composition that works best for a particular population of individuals with respect to the muscle and/or bone growth as determined from a stratification profile based upon using the assay described herein can be selected and administered to the subject. In other embodiments, other factors such as side effects and/or price of the drug, and/or other drugs that the subject is taking can be considered when selecting the test composition for treating the subject. In such embodiments, the test composition with a lower rank and an anabolic efficacy above a pre-determined threshold (e.g., anabolic response of the musculoskeletal or precursor cells thereof in the absence of the test composition) can be selected and administered to the subject instead.

Not only can the anabolic profiles generated by the assay described herein provide personalized or stratified information about which test composition indicates a higher anabolic efficacy for a specific subject or a subset of population, but it can also determine anabolic resistance of the specific subject or the subset of population. For example, if a subset of the test compositions associated with a specific anabolic pathway score a low rank and/or do not reach a pre-determined threshold value of anabolic efficacy, it indicates that the specific subject or the subset of population can develop an anabolic resistance to the molecules associated with the specific anabolic pathway. Accordingly, methods for determining an anabolic resistance in a subject or a subset of populations are also provided herein. The method comprises subjecting the musculoskeletal cells or precursor cells thereof obtained or derived from a subject or a subset of populations to one or more embodiments of the assay described herein. When the anabolic efficacy of at least one of the test compositions is determined to be below a pre-determined threshold, it indicates that the subject is or the subset of the population are non-responsive or resistant to the at least one of the test compositions.

In some embodiments, the methods of various aspects described herein do not necessarily require a biological sample from a subject to perform the assay as described herein. Instead, a database comprising anabolic profiles for a plurality of population subgroups stratified by at least one feature such as phenotypic feature can be created and established. Thus, a subject seeking an anabolic treatment can be matched to one of the population subgroups in the database based on at least one feature such as phenotypic feature (e.g., age, gender, ethnicity, condition and/or BMI), thereby selecting an anabolic agent based on the rankings of the anabolic agents in the matching population subgroup. Accordingly, in another aspect, provided herein is a method of selecting an anabolic agent for a subject in need of anabolic augmentation and/or mitigation of muscle and/or bone loss. The method comprises (a) creating a database comprising anabolic information for a plurality of population subgroups stratified by at least one feature, wherein the anabolic information for each of the population subgroups comprises rankings of a plurality of anabolic agents based on their anabolic efficacy in each of the population subgroups; and (b) mapping a subject who is in need of anabolic augmentation or muscle loss reduction to one of the plurality of the population subgroups based on the at least one phenotypic feature, thereby selecting at least one anabolic agent for the subject based on the ranking of the anabolic agents in the matching population subgroup. Examples of a feature can be a phenotypic feature to stratify the population subgroups including, but not limited to, age groups, gender, ethnicity, condition, body types, body mass index (BMI), blood types, activity levels, chronic diseases, acute diseases, genetic polymorphisms, diet, drug resistance, treatment regime such as chemotherapy, drastic/abnormal weight loss, geographical location, and any combinations thereof. In some embodiments, the subject can be mapped or associated to one of the population subgroups based on age and gender.

In some embodiments, the method can further comprise administering to the subject the selected anabolic agent. Accordingly, provided herein is also a method of treating a subject who is in need of anabolic augmentation and/or mitigation of muscle and/or bone loss, which comprises administering at least one selected anabolic agent to the subject, wherein the at least one selected anabolic agent is determined based on a process comprising: (a) providing a database comprising anabolic information for a plurality of population subgroups stratified by at least one feature such as a phenotypic feature, wherein the anabolic information for each of the population subgroups comprises rankings of a plurality of anabolic agents based on their anabolic efficacy in each of the population subgroups; and (b) mapping the subject to one of the plurality of the population subgroups based on the at least one feature such as the phenotypic feature, thereby selecting the at least one anabolic agent for the subject based on the ranking of the anabolic agents in the matching population subgroup.

In some embodiments, the anabolic efficacy of the anabolic agents can be determined based on the effect of the anabolic agents on fusion of muscle precursor cells to form multi-nucleated cells. Additionally or alternatively, the anabolic efficacy of the anabolic agents can be determined based on the effect of the anabolic agents on differentiation of muscle cells or bone precursor cells to bone cells. Accordingly, in some embodiments, the database can be created by a method comprising: (a) for each of the plurality of the population subgroups, quantifying muscle growth and/or bone growth of the musculoskeletal cells or precursor cells thereof obtained or derived from the population subgroup, upon the contact of the musculoskeletal cells or precursor cells thereof with the plurality of the anabolic agents; and (b) ranking anabolic efficacy of the plurality of the anabolic agents based on the quantified muscle growth and/or bone growth for each of the plurality of the population subgroups.

The methods of any aspects described herein can be used to facilitate the treatment and/or prevention of any musculoskeletal disorder or disease. Examples of a musculoskeletal disorder or disease can include, but are not limited to, muscle loss, muscle wasting, muscle wasting associated with HIV infection, muscle wasting in cancer survivors, cachexia, muscular dystrophy, osteopenia, osteoporosis, sarcopenia, an age-related musculoskeletal disease or disorder, or a musculoskeletal disease or disorder associated with anabolic resistance, a musculoskeletal disease or disorder associated with excessive weight loss, or any combinations thereof.

In some embodiments, the subjects amenable to the methods of any aspects described herein can include, but are not limited to, individuals suffering or having a risk for a musculoskeletal disease or disorder, athletes, aging individuals, individuals having a chronic disease or disorder (e.g., but not limited to, cancer, chronic obstructive pulmonary disease (COPD), chronic kidney disease (CKD), chronic liver failure (CLF), and chronic infections), individuals suffering from malnutrition, individuals afflicted with HIV infection, cancer survivors, individuals showing excessive weight loss, individuals that have previously shown non-responsiveness or resistance to at least one or more anabolic agents, or any combinations thereof.

Computer systems for use in any aspects of the assays and/or methods described herein are also provided. For example, one embodiment provided herein is a computer system for generating anabolic profiles for at least one or more subjects. The computer system comprises: (a) a determination module configured to receive at least one or more samples each comprising a population of the musculoskeletal cells or precursor cells thereof and perform the following steps: (i) contacting the musculoskeletal cells or precursor cells thereof with a plurality of test compositions each comprising at least one agent selected to increase and/or maintain muscle and/or bone growth; and (ii) subjecting the musculoskeletal cells or precursor cells thereof to at least one analysis (including, e.g., at least two analyses) to quantify muscle growth and/or bone growth of the musculoskeletal cells or precursor cells thereof in response to the test compositions; (b) a storage device configured to store data output from said determination module; and (c) an analysis module configured to rank anabolic efficacy of the test compositions based on the data output from said determination module; and (d) a display module for displaying a content based in part on the data output from said determination module. The content displayed in the display module can comprise a signal indicative of at least a partial ranking of the anabolic efficacy of the test compositions, or a signal indicative of at least one or more test compositions recommended for the subject's treatment, or a signal indicative of no test composition recommended for the subject.

A sample received by the determination module can contain musculoskeletal cells or precursor cells thereof obtained or derived from a biological sample (e.g., a muscle biopsy or a blood sample) of a subject who is seeking an anabolic treatment. Alternatively, a sample can contain musculoskeletal cells or precursor cells thereof obtained or derived from a panel of tissue specimens or cells representing one or more different population subgroups. The panel of tissue specimens or cells representing one or more different population subgroups can be obtained from a tissue or cell depository. In some embodiments, the musculoskeletal cells or precursor cells thereof can contain cells from individuals that share at least one feature such as a phenotypic feature (e.g., but not limited to, age, gender, BMI, condition, and ethnicity). For example, in one embodiment, the musculoskeletal cells or precursor cells thereof can contain cells from different population subgroups characterized by age or age groups and gender. In some embodiments, a sample can contain musculoskeletal cells or precursor cells thereof obtained or derived from a subject who is determined to have or have a risk for a musculoskeletal disease or disorder described herein.

The determination module can be configured in any manner to accommodate different types of analyses selected to quantify muscle growth and/or bone growth of the musculoskeletal or precursor cells thereof. In some embodiments, the determination module can be configured to determine the number of multi-nucleated cells formed by fusion of mononucleated musculoskeletal cells or precursor cells thereof for quantifying muscle growth. For example, the determination module can be configured to include a microscope and an imaging system that permit examining and/or capturing images of the musculoskeletal cells or precursor cells thereof for muscle growth analysis (e.g., quantifying formation of multi-nucleated cells and/or fusion of mononucleated muscle cells). In some embodiments, the determination module can be further configured to determine the number of bone cells differentiated from the musculoskeletal cells or precursor cells thereof (e.g., muscle cells or bone precursor cells) for quantifying bone growth. By way of example only, the determination module can be configured to perform immunostaining, protein expression analysis, and/or nucleic acid expression analysis on the cells, e.g., to detect the bone cells based on expression of a bone marker. In one embodiment, the bone marker is alkaline phosphatase (ALP). Other examples of a bone marker can include, but are not limited to type I collagen propetides and/or osetocalcin. The images and/or data collected by the determination module can be stored in the storage device for subsequent analyses.

In some embodiments, the analysis module can comprise at least one image analysis algorithm to quantify muscle growth and/or bone growth based on the images of cells captured by the determination module and stored in the storage device. The image analysis algorithm can be programmed to quantify the number of multi-nucleated cells formed by fusion of mononucleated musculoskeletal cells or precursor cells thereof in each image. Alternatively or additionally, the image analysis algorithm can be programmed to quantify the number of bone cells present in each image, e.g., based on expression of a bone marker described herein.

In some embodiments, the analysis module can further comprise a comparison algorithm adapted to compare the data output from the determination module with reference data stored on the storage device. The reference data can include anabolic data (e.g., muscle and bone growth) from a negative control (e.g., in the absence of the test composition(s)); anabolic data (e.g., muscle and bone growth) from a positive control (e.g., in the presence of an anabolic agent that is well known to stimulate muscle and/or bone growth); anabolic data (e.g., muscle and bone growth) of one or more subjects from at least one previous time point; and/or anabolic data (e.g., muscle and bone growth) of one or more normal healthy subjects without any known muscle or bone loss.

A computer readable physical medium having computer readable instructions recorded thereon to define software modules for implementing a method on a computer is also described herein. The computer readable storage medium comprises: (a) instructions for analyzing the data stored on a storage device that in part comprises data indicative of anabolic responses of musculoskeletal cells or precursor cells thereof to a plurality of test compositions comprising at least one agent selected to increase and/or maintain muscle and/or bone growth; wherein the data analysis ranks anabolic efficacy of the test compositions based on the data stored on the storage device; and (b) instructions for displaying a content based in part on the data stored on the storage device. The content to be displayed can comprise a signal indicative of at least a partial ranking of the anabolic efficacy of the test compositions, or a signal indicative of at least one test composition recommended for the subject's treatment, or a signal indicative of no test composition recommended for the subject.

Provided herein is also a processor-readable medium including instructions that, when executed by a processing device, cause the processing device to perform a method comprising: (a) receiving subject-specific information comprising at least one feature such as a phenotypic feature; (b) mapping, by the processing device, a subject to one of a plurality of population subgroups in a database based on the at least one feature such as the phenotypic feature, wherein the database comprises anabolic information for the plurality of the population subgroups stratified or characterized by the at least one feature such as the phenotypic feature, and wherein the anabolic information for each of the population subgroups comprises rankings of a plurality of anabolic agents based on their anabolic efficacy in each of the population subgroups; and (c) displaying a content based in part on the anabolic information of the matching population subgroup, wherein the content comprises a signal indicative of at least a partial ranking of the anabolic efficacy of the anabolic agents, or a signal indicative of at least one anabolic agent recommended for the subject, or a signal indicative of no anabolic agent recommended for the subject. The content can be displayed on a screen, a monitor, or paper. In some embodiments, the processing device can be a personal digital assistant (PDA), smart-phone, cellular telephone, a computer, a tablet PC, or any combinations thereof.

A further aspect provides kits that can be used in the assays, systems, and methods of any aspects described herein. For example, in some embodiments, the kits can be used to generate a personalized diagnostic report that ranks each subject's response to the test compositions. In other embodiments, the kits can be used as diagnostic kits for optimizing or selecting an anabolic treatment of a musculoskeletal disease or disorder. In one embodiment, a kit comprises (a) a plurality of test compositions each comprising at least one agent selected to maintain and/or increase muscle and/or bone growth; (b) a first container containing a first substrate material optimized for muscle growth and/or differentiation; and optionally (c) a second container containing a second substrate material optimized for bone growth and/or differentiation.

In some embodiments, the first substrate material and the second substrate material can be pre-aliquoted or disposed into individual wells of a micro-titer plate for cell culture. In other embodiments, the first substrate material and the second substrate material can be contained in a vial or a tube.

In some embodiments, each of the test compositions can be pre-distributed into individual wells of a micro-titer plate for cell culture. In some embodiments, the test compositions can be each pre-mixed into individual aliquots of the first and second substrate material.

In some embodiments, the kit can further comprise at least one micro-titer plate. In some embodiments, the kit can further comprise at least one reagent, e.g., but not limited to, cell culture medium, a cell stain (e.g., DAPI), an agent for detecting a bone marker (e.g., an antibody to a bone marker such as ALP).

In some embodiments, the kit can further comprise an agent to facilitate purification or isolation of muscle cells or precursor cells thereof from a subject's specimen (e.g., a muscle biopsy or a blood sample). For example, anti-CD45 and anti-CD46 magnetic beads can be included in the kit for use in purification or isolation of muscle cells from a muscle biopsy. In another embodiment, the kit can be used with a blood sample. Using induced pluripotent stem (iPS) cell technology, blood cell-derived muscle and bone cells are then used to generate patient specific muscle and bone cells for ex vivo therapeutics. In these embodiments, the kit can further comprise stem cell differentiation factors to generate iPS cells.

### BRIEF DESCRIPTION OF THE DRAWINGS

FIG. 1 is a schematic of an exemplary protocol for obtaining muscle anabolic profile using muscle cells from individual subjects. In step 1, cells are purified using CD45 and CD56-identifying molecules. Cells are generally all mononucleated (one nucleus per cell, as indicated in the image and graph). In step 2, cells are plated in a specific ECM scaffold and treated with an array of anabolic compounds. About 48 hours later, cells are stained with DAPI and nuclei are then visualized and digitally recorded to quantify fusion index and determine rank of response for each subject.

FIGS. 2A-2B shows partial results of an exemplary muscle anabolic or myogenic screen of known and novel anabolic compounds that promote muscle growth. In FIG. 2A, the left panel indicates results of a test plate with a grid of wells that contain muscle cells simultaneously exposed to different compounds from an FDA library and other compound libraries. The number within each well is a score indicating the potency of each compound in stimulating muscle growth. The right panel shows an example of one well, D14, which displays a potency index or fusion index of 4.0 (compared with untreated 0.0), a metric that indicates cells with 3 or more nuclei (nuclei are shown in darker shades within cells appearing translucent). FIG. 2B shows examples of anabolic compounds (e.g., pro-muscle compounds) identified in the muscle anabolic (myogenic) screen, and the corresponding well identification in the plate as shown in FIG. 2A.

FIG. 3 shows an example of brightfield images of muscle cells in response to an anabolic compound promoting bone growth. Cells were treated with media (Negative control), BMP-2 (Positive control), or a combination of a compound (e.g., compound 92) and BMP-2 for about 48 hours, and then assayed for ALP staining. The left panel is an image showing no ALP signal from a negative control. The center panel is an image showing induction of ALP in positive-control cells, e.g., cells in the presence of BMP-2. The right panel is an image showing robust induction of ALP in the presence of compound 92.

FIGS. 4A-4B show a schematic of an exemplary protocol for high-throughput screening of small molecules to investigate BMP-2 induced promotion of osteoblast formation. FIG. 4A shows an overview of C2C12 cells switched to differentiation media (DM) (low serum) and that received either no BMP-2 (negative control), BMP-2 only (positive control), or BMP-2+compound*. The level of alkaline phosphatase (ALP) activity measured colorimetrically was scored in all wells using three criteria (see Exemplary materials and methods in the Examples section). Compounds that enhanced ALP expression were considered for further analysis. FIG. 4B is a schematic of 5405 compounds tested on C2C12 cells in a 384-well plate format, in duplicate for BMP-2 induced ALP expression*. ALP intensity images were acquired using a Digilab Plate reader. Images were analyzed using three independent criteria and considered for secondary screening and validation in the MC3T3 pre-osteoblast cell line Enhancement of the mRNA and protein levels of the mature osteoblast markers RunX2 and osterix was tested. *Plates were run in duplicate.

FIG. 5 is a Venn diagram depiction of the 3 analysis approaches. ImageJ analysis was used to find compounds that were three standard deviations above the positive controls. 211 compounds were identified with ImageJ. Digilab analysis was used to find compounds that were in the 95th percentile and 31 compounds were identified under this category. The compounds were also analyzed by visual inspection with 44 noted. Of these, 18 compounds were common to all three analyses. Functional categories of the 18 compounds are indicated in the inset box.

FIGS. 6A-6B show that rapamycin and FK-506 increase BMP-2 induced phosphorylation of Smad 1/5/8. MC3T3 pre-osteoblast cells were plated at a density of 8×10⁵ cells per 9.6 cm² well. BMP-2 was added to differentiate the cells with rapamycin (FIG. 6A) or FK-506 (FIG. 6B) at a concentration of 100 ng/mL. Total protein was collected at 5 and 10 min and analyzed by western blot with antibodies to phospho-Smad 1/5/8 and total Smad 1/5/8. Graphs are shown as a ratio of phosphorylated and total Smad 1/5/8 and compared to untreated samples. Shown are representative results (average of duplicates) of at least three independent experiments. (*indicates p-value=0.001).

FIGS. 7A-7B show that rapamycin and FK-506 increase BMP-2 induced Runx2 and Osx transcripts. MC3T3 pre-osteoblast cells were plated at a density of 8×10⁵ cells per 9.6 cm² well. BMP-2 was added to differentiate the cells with rapamycin (FIG. 7A) or FK-506 (FIG. 7B) at a concentration of 100 ng/mL. Cells were harvested at 6 h and 24 h time points, RNA was purified and target transcripts Runx-2 and Osx were analyzed by qRT-PCR. UT=untreated and Stim=stimulated with the indicated compound. Values represent fold change compared to untreated samples after normalization using the ΔΔCT method. Shown are representative results (average of duplicates) of at least three independent experiments. (*indicates p-value=0.001 for both Runx2 and Osx).

FIGS. 8A-8B show that rapamycin and FK-506 induce osteoblast differentiation independently of BMP-2. The same experiments were done as shown in FIGS. 6A-6B and FIGS. 7A-7B, except rapamycin and FK-506 were added to the MC-3T3 cells without BMP-2. (FIG. 8A) Phosphorylation of Smad 1/5/8 was observed at 5, 10 and 30 min after stimulation and compared to total Smad 1/5/8 levels via western blot. The graph represents the ratio of P-Smad to total Smad and is representative of 3 independent experiments. (FIG. 8B) RNA was collected 24 h after simulation and analyzed by qRT-PCR for Runx2 and Osx levels. The values for the graph were determine by ΔΔCT and compared to the untreated sample and were normalized to 18S. This was representative of three independent experiments (*indicates p-value=0.02 for both Runx2 and Osx).

FIGS. 9A-9B show that FK-506 induces late differentiation markers. MC-3T3 cells were plated at a density of 8×10⁵ cells per 9.6 cm² well and treated with 100 ng/mL FK-506 in the presence or absence of 100 ng/mL BMP-2. (FIG. 9A) Media were replaced with fresh compound stimulation every two days and then RNA was collected and purified for qRT-PCR of Ocn mRNA transcripts on day 14. Values in the graph represent fold change compared to untreated samples after normalization using the ΔΔCT method. Shown are representative results (average of duplicates) of at least three independent experiments. (*indicates p-value=0.001). (FIG. 9B). Media were replaced with fresh compound simulation every two days and then stained on day 21 with Alizarin-Red. Pictures are representative of three independent experiments. Quantification was done using ImageJ (*indicates p-values=0.02).

FIGS. 10A-10B show that TGFβ inhibits osteoblast differentiation and rapamycin rescues this inhibition while increasing induction of Smad 7 transcripts. MC3T3 cells were treated with media containing 1 ng/mL of TGFβ1 for 24 h and then the media were replaced with BMP-2 or BMP-2 with 100 ng/mL rapamycin. RNA was collected 24 h afterwards and analyzed by qRT-PCR and the ΔΔCT method. (FIG. 10A) Values of Runx2 and Osx transcripts are represented as fold change compared with untreated samples (*indicates p-value=0.02). (FIG. 10B) Values of Smad 7 transcripts. Shown are representative results (average of duplicates) of at least three independent experiments. (*indicates p-value=0.03).

FIG. 11 is a graphical model of role for rapamycin and FK-506 promoting osteoblastogenesis.

FIG. 12 is a block diagram showing an example of a system for determining anabolic profiles from a population of musculoskeletal cells or precursor cells thereof obtained from at least one subject.

FIG. 13 is a block diagram showing exemplary instructions on a computer readable medium for assessing anabolic profiles of a subject, e.g., to optimize or select a treatment regimen for the subject determined to have a musculoskeletal disease or disorder.

FIG. 14 is a schematic diagram showing an exemplary process of generating a personalized anabolic profile based on a personal biopsy or a panel of cells representing a diverse set of individuals or a panel of cells representing a population subgroup that shares at least one feature such as a phenotypic feature (e.g., but not limited to, age, gender, condition, and ethnicity) with a subject in need of muscle augmentation or mitigation of muscle and/or bone loss.

### DETAILED DESCRIPTION OF THE INVENTION

Human genetic variation and life history influence, often unpredictably, the response to therapeutic intervention for treatment of a musculoskeletal disease or disorder, such as HIV-associated musculoskeletal disease or disorder. Current diagnostic and treatment procedures do not distinguish between different types and/or causes of musculoskeletal decline and do not provide guidance for anabolic therapy specific to an individual. For example, current diagnostic options for evaluating muscle loss include measuring blood levels of creatine kinase (CK), which is an indirect measure of muscle loss that may occur in response to many non-muscle pathologies. Additional diagnostics include body composition analysis using CT or MRI imaging to qualitatively evaluate muscle tissue. The current standard of care for muscle wasting is to provide nutritional supplementation and off-label prescriptions for anabolic agents. While existing diagnostics are used to identify muscle loss, they do not provide targeted decision support for patients to evaluate which, among the many treatment options available, are more effective for their unique anabolic needs. Accordingly, there is a need for development of assays and/or kits that can provide a more personalized, and scalable, anabolic guide to optimize treatment of musculoskeletal wasting, improve health outcomes and/or reduce healthcare costs associated with musculoskeletal decline in muscle and bone disease as well as in aging.

Various aspects provided herein generally relate to assays, methods, systems, and kits, e.g., for profiling anabolic responses such as skeletal muscle and bone cell growth of individuals (e.g., a mammalian subject such as a human) or different population subgroups in response to a panel of anabolic compounds. The assays, methods, systems, and kits described herein are, in part, based on ranking relative abilities of various anabolic compounds (including candidate agents or compositions to be assessed for their anabolic effects) to stimulate muscle and/or bone growth of subject-specific cells or patient-specific cells, e.g., collected from a biological sample (e.g., a muscle microbiopsy or a blood sample), or of a panel of cells representing different population subgroups, thus generating a personalized or stratified anabolic diagnostic report. In some embodiments, the panel of cells representing different population subgroups can be stratified based one or a plurality of (e.g., at least two or more) feature(s) of the population subgroups. Examples of a feature can be a phenotypic feature for population stratification including, but not limited to, age groups, gender, ethnicity, body types, body mass index (BMI), blood types, condition, activity levels, chronic diseases, acute diseases, genetic polymorphisms, diet, drug resistance, treatment regime such as chemotherapy, drastic/abnormal weight loss, geographical location, and any combinations thereof. The generated subject-specific or stratified anabolic profiles can be used to make therapeutic decisions, e.g., selecting a test composition for treating and/or preventing muscle and/or bone loss or a musculoskeletal disease or disorder in the subject, based on the ranking of the test composition in one or more embodiments of the assay described herein. Thus, in some embodiments, an anabolic agent or composition can be selected, recommended and/or optionally administered to a subject or patient based on a subject-specific or patient-specific anabolic profile generated using his/or own biopsy and/or blood sample. Alternatively, an anabolic agent or composition can be selected, recommended, and/or taken or otherwise administered to a subject or patient based on a stratified anabolic profile generated using tissue specimens of a matching population subgroup as the subject, based on one or a plurality of pre-determined feature such as phenotypic feature(s), e.g., but not limited to, age, gender, ethnicity, condition, and/or body mass index (BMI). By way of example only, for a 30-year old diabetic Caucasian woman who is in need of muscle augmentation and/or mitigation of muscle or bone loss, an optimal anabolic agent or composition can be selected for this woman, based on a personalized anabolic profiling (which requires her own biopsy or blood sample), and/or a stratified anabolic profiling of a population subgroup of about 30-year old (e.g., 25-35-year old) diabetic Caucasian women. Factors can also be based on whether the subject is taking the anabolic agent in response to the subject's circumstance or condition such as chemotherapy or massive and sudden weight loss.

Unlike the existing diagnostic methods such as CT and MRI or blood biomarkers such as creatine phosphor-kinase (CPK), embodiments of the assays, methods, systems, and kits described herein can provide information about muscle and/or bone function in response to a variety of test compositions, which can be in turn used to make diagnostic, and/or therapeutic or prophylactic decisions. For example, in some embodiments, the generated functional anabolic profiles can be used to diagnose an anabolic deficiency, and/or a defect in and/or an imbalance among anabolic growth pathway(s) in a subject. In some embodiments, the generated functional anabolic profiles can be used to identify and/or optimize a therapeutic option for treatment of a muscle wasting-associated disease or disorder. In other embodiments, the generated functional anabolic profiles can be used to identity and/or optimize a prophylactic option to prevent or mitigate muscle loss and to optimize and maintain muscle heath. Accordingly, methods for diagnosing, treating and/or preventing muscle wasting or a musculoskeletal disease or disorder in a subject are also provided herein. Methods for determining a risk for anabolic resistance or potential anabolic resistance in a subject are also described herein.

### Cell-Based Assays

One aspect provided herein relates to cell-based assays using musculoskeletal cells or precursor cells thereof to generate anabolic profiles specific for individual subjects (personalized anabolic profiles) or representing different population subgroups (stratified anabolic profiles). The assay comprises: (a) contacting a population of musculoskeletal cells or precursor cells thereof with a plurality of test compositions (e.g., at least two or more test compositions) to profile anabolic responses of the cells to the test compositions, wherein each of the test compositions comprises at least one agent selected to maintain and/or increase muscle and/or bone growth; (b) subjecting the musculoskeletal cells or precursor cells thereof to at least one analysis to quantify muscle growth or bone growth of the musculoskeletal cells or precursor cells thereof in response to the test compositions; and (c) ranking anabolic efficacies of the plurality of the test compositions based on the quantified muscle growth and/or bone growth, thereby providing anabolic profiles (e.g., muscle anabolic profiles and/or bone anabolic profiles) of the assayed cells. In some embodiments, the musculoskeletal cells or precursor cells thereof are subjected to at least two analyses to quantify their muscle growth and bone growth in response to the test compositions.

As used herein, the term “anabolic profile” refers to anabolic responses of the musculoskeletal cells or precursor cells thereof to a variety of anabolic agents or compositions. The anabolic responses of the musculoskeletal cells or precursor cells thereof can be characterized by quantifying muscle growth and/or bone growth of the cells as described in detail below. In some embodiments, muscle growth of the musculoskeletal cells or precursor cells thereof can be characterized by formation of multi-nucleated muscle cells (e.g., individual muscle cells each containing at least two or more nuclei). In some embodiments, bone growth of the musculoskeletal cells or precursor cells thereof can be characterized by formation of bone cells.

In some embodiments, the assay can be used to generate personalized anabolic profiles for individual subjects or patients. Accordingly, in some embodiments, the musculoskeletal cells or precursor cells thereof for use in the assay described herein can be obtained or derived from a biological sample of a subject or patient seeking for an anabolic treatment or supplement. For example, the musculoskeletal cells or precursor cells thereof can be obtained or derived from a muscle biopsy or microbiopsy and/or a blood sample of a subject or patient. In one embodiment, muscle stem cells derived from a subject's biopsy or blood sample can be subjected to the assay described herein. Thus, the generated anabolic profile for muscle growth and/or bone growth can be personalized to the specific subject or patient.

In some embodiments, the assay can be used to generate stratified anabolic profiles for distinct population subgroups. In these embodiments, the musculoskeletal cells or precursor cells thereof for use in the assay described herein can be obtained or derived from cells or tissue specimens representing one or different population subgroups. The cells or tissue specimens representing one or more different population subgroups can be obtained from a cell or tissue depository. As used herein, the term “population subgroups” refers to subsets or subgroups of a population stratified by at least one or more (including, e.g., at least two, at least three, at least four or more) feature of the population. Examples of a feature can be a phenotypic feature for population stratification including, but not limited to, age or age groups, gender, ethnicity or races, body types, weights, heights, body mass index (BMI), blood types, activity levels (e.g., sedentary lifestyle or work such as a secretary in office vs. active lifestyle or work such as an athlete), a condition such as chronic or acute diseases (e.g., but not limited to, diabetes, cancer, osteoporosis, HIV infection, infection, musculoskeletal diseases or disorders, metabolic diseases or disorders, and psychophysiological disorders), genetic polymorphisms, diet (e.g., but not limited to, vegetarian, and gluten-free), living habits (e.g., but not limited to, smoking and alcohols), drug resistance, treatment regime such as chemotherapy, drastic weight loss, geographical location (e.g., individuals living in the west coast vs. east coast of the United States, or individuals living in the United States vs. in Asian countries) and environmental factors associated therewith, and any combinations thereof. By way of example only, in one embodiment, population subgroups can be stratified by age or age groups, gender, ethnicity, body mass index (BMI), and any combinations thereof. In some embodiments, a stratified anabolic profile of a population subgroup that shares at least one or more phenotypic features with a subject seeking for an anabolic treatment and/or supplement can be used to determine an optimal treatment and/or supplement for the subject. One can create bands or population subgroups based upon sex (gender) and age. The age groupings can be 20 years, 15 years, 10 years, 5 years, etc. One can group women based upon whether they are in a child-bearing years or not, pregnant or not, under 21 years, or over 50 years of age, etc.

As used herein, the term “contacting” refers to any suitable means for delivering, or exposing, an agent or a test composition to at least one cell in vitro. Exemplary delivery methods include, but are not limited to, direct delivery to cell culture medium, delivery to an in vitro substrate material (e.g., an extracellular matrix (ECM) scaffold) in which cells are seeded, e.g., via perfusion or injection, or other delivery method well known to one skilled in the art. In one embodiment, a test composition is added to the cell culture medium in which the musculoskeletal cells or precursor cells thereof are cultured. In another embodiment, a test composition is distributed or mixed into a substrate material (e.g., an ECM scaffold) in which the musculoskeletal cells or precursor cells thereof are placed. The term “treatment” or “treated” as used herein, with respect to exposing cells to an agent, e.g., cells treated with an agent, is used herein interchangeably with the term “contacting.”

The contact of the musculoskeletal cells or precursor cells thereof with a plurality of test compositions (e.g., 2, 3, 4, 5, 6, 7, 8, 9, 10, 20, 30, 40, 50, 100, 150, 200, 300, 400, or more) can be performed in vitro in any assay container. For example, in one embodiment, the musculoskeletal cells or precursor cells thereof are placed in a multi-well microtiter plate (e.g., 48-well plate, 96-well plate, 384-well plate or 1536-well plate), wherein the cells in each well are contacted with one test composition, except where the cells are used as a negative control (e.g., in the absence of a test composition).

In order to determine specific anabolic responses (e.g., muscle growth and/or bone growth) of the musculoskeletal cells or precursor cells thereof to one or more test compositions, the cells can be cultured in separate conditions optimized for muscle growth and/or bone growth. For example, to determine the muscle growth-response of the musculoskeletal cells or precursor cells thereof to a plurality of test compositions, it can be desirable to culture and/or maintain the cells in a muscle cell-specific condition (e.g., a condition optimal to muscle differentiation) during the contact with the test compositions. In some embodiments, the muscle cell-specific condition can include culturing in a substrate material, which is further described in detail below, for example, with a defined stiffness optimal to muscle differentiation such as a stiffness of about 5 kPa to about 50 kPa, or about 10 kPa to about 20 kPa.

To determine the bone growth-response of the musculoskeletal cells or precursor cells thereof to a plurality of test compositions, it can be desirable to culture and/or maintain the cells in a bone cell-specific condition (e.g., a condition optimal to bone differentiation) during the contact with the test compositions. In some embodiments, the bone cell-specific condition can include culturing in a substrate material, which is further described in detail below, e.g., with a defined stiffness optimal to bone differentiation such as a defined stiffness of about 10 kPa to about 150 kPa, or about 20 kPa to about 100 kPa. While not necessary, in some embodiments, the bone cell-specific condition can further include culturing in the presence of a bone formation-inducing agent. An exemplary bone formation-inducing agent includes, but not limited to, bone morphogenetic protein-2 (BMP-2).

The musculoskeletal cells or precursor cells thereof can be contacted with different test compositions for any period of time, e.g., minutes, hours, or days. In some embodiments, the population of the musculoskeletal cells or precursor cells thereof can be contacted with a plurality of test compositions for at least about 6 hours, at least about 12 hours, at least about 18 hours, at least about 24 hours, at least about 2 days, at least about 3 days, at least about 4 days, at least about 5 days, at least about 6 days, at least about 7 days or longer. In some embodiments, the population of musculoskeletal cells or precursor cells thereof can be contacted with a plurality of test compositions for at least about 48 hours or longer. In some embodiments, the population of musculoskeletal cells or precursor cells thereof can be contacted with a plurality of test compositions for at least about 1 week, at least about 2 weeks, at least about 3 weeks, at least about 4 weeks or longer.

In some embodiments, the musculoskeletal cells or precursor cells thereof can be contacted with a test composition for an amount of time sufficient to increase muscle growth, e.g., by at least about 10% or more, as compared to the musculoskeletal cells or precursor cells thereof in the absence of the test composition. For example, in some embodiments, the musculoskeletal cells or precursor cells thereof can be contacted with a test composition for an amount of time sufficient to increase muscle growth by at least about 10%, at least about 20%, at least about 30%, at least about 40%, at least about 50%, at least about 60%, at least about 70%, at least about 80%, at least about 90%, at least about 95% or more, as compared to the musculoskeletal cells or precursor cells thereof in the absence of the test composition. In some embodiments, the musculoskeletal cells or precursor cells thereof can be contacted with a test composition for an amount of time sufficient to increase muscle growth by at least about 1.5-fold, at least about 2-fold, at least about 3-fold, at least about 4-fold, at least about 5-fold, at least about 6-fold, or longer, as compared to the musculoskeletal cells or precursor cells thereof in the absence of the test composition. Methods for determining muscle growth are known in the art, for example, but not limited to, microscopic imaging, nucleic staining, cell staining, protein expression, nucleic acid expression, and any combinations thereof. By way of example only, muscle growth can be detected by examining the morphological change of mononucleated muscle cells or muscle precursor cells to form multi-nucleated muscle cells (e.g., muscle cells each independently having at least 2 nuclei, at least 3 nuclei, or more). In some embodiments, the nuclei of the cells can be stained in situ and imaged using a high throughput analysis instrument, e.g., a microscopic imaging system. Thus, in one embodiment, the muscle growth of the musculoskeletal cells or precursor cells thereof induced by a test composition can be quantified by an increase in the number of multi-nucleated cells formed by fusion of the musculoskeletal cells or precursor cells thereof, as compared to muscle growth in the absence of the test composition. Nuclei distribution (e.g., the number of nucleic per cell distribution) can be generated for each test composition and the relative ranks of the test compositions for promoting muscle growth can then be ranked to generate a muscle anabolic profile.

In some embodiments, the musculoskeletal cells or precursor cells thereof can be contacted with a test composition for an amount of time sufficient to increase bone growth, e.g., by at least about 10% or more, as compared to the musculoskeletal cells or precursor cells thereof in the absence of the test composition. For example, in some embodiments, the musculoskeletal cells or precursor cells thereof can be contacted with a test compositions for an amount of time sufficient to increase bone growth by at least about 10%, at least about 20%, at least about 30%, at least about 40%, at least about 50%, at least about 60%, at least about 70%, at least about 80%, at least about 90%, at least about 95% or longer, as compared to the musculoskeletal cells or precursor cells thereof in the absence of the test composition. In some embodiments, the musculoskeletal cells or precursor cells thereof can be contacted with a test composition for an amount of time sufficient to increase bone growth by at least about 1.5-fold, at least about 2-fold, at least about 3-fold, at least about 4-fold, at least about 5-fold, at least about 6-fold, or longer, as compared to the musculoskeletal cells or precursor cells thereof in the absence of the test composition. By way of example only, bone growth can be determined by detection of bone or osteoblast phenotype cells differentiated from precursor cells thereof such as muscle cells, muscle stem cells, and/or bone precursor cells. Thus, in one embodiment, the bone growth of the musculoskeletal cells or precursor cells thereof induced by a test composition can be quantified by an increase in the number of bone or osteoblast phenotype cells differentiated from the musculoskeletal cells or precursor cells thereof (e.g., muscle cells, muscle stem cells, and/or bone precursor cells), as compared to bone growth in the absence of the test composition. Methods of identifying bone or osteoblast phenotype cells are known in the art, and such exemplary methods are described in detail below, including, e.g., detection of a bone or osteoblast phenotype cell by expression of a bone marker (e.g., but not limited to, alkaline phosphatase).

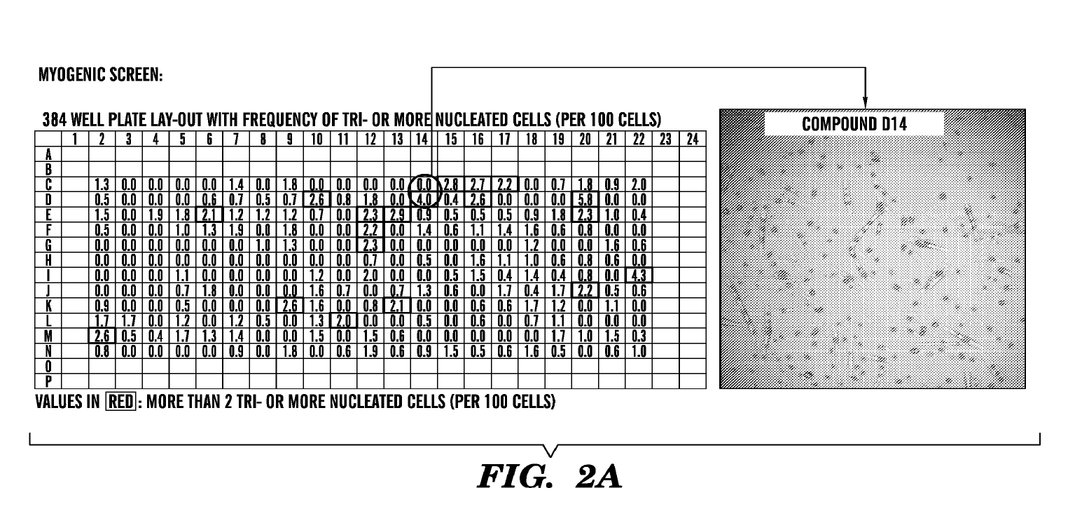

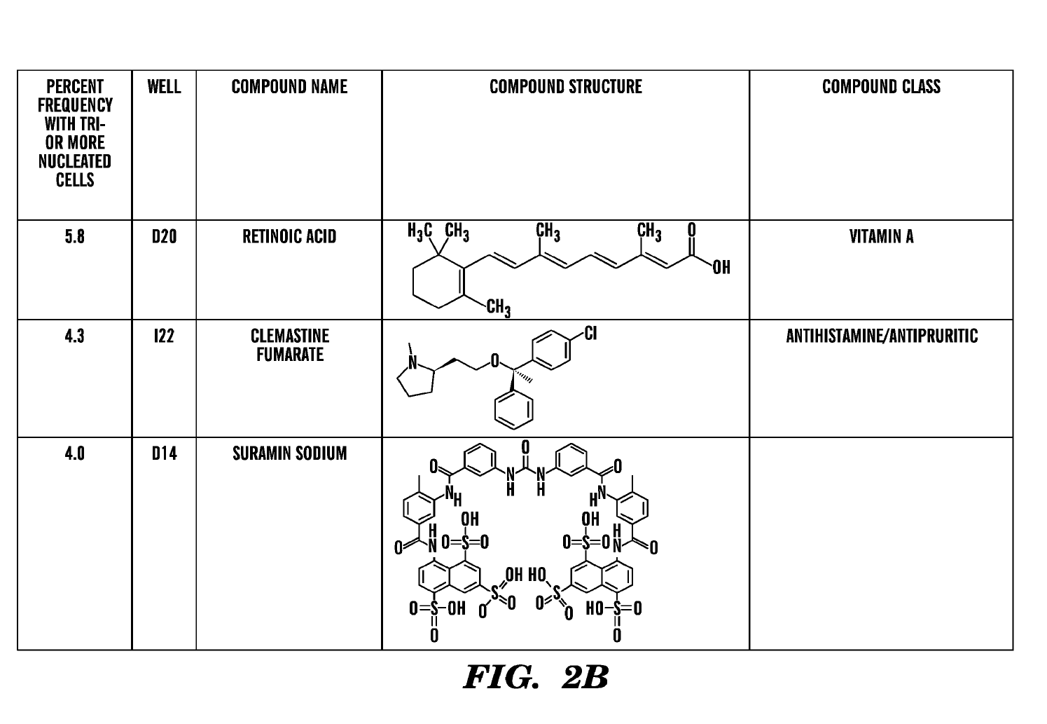

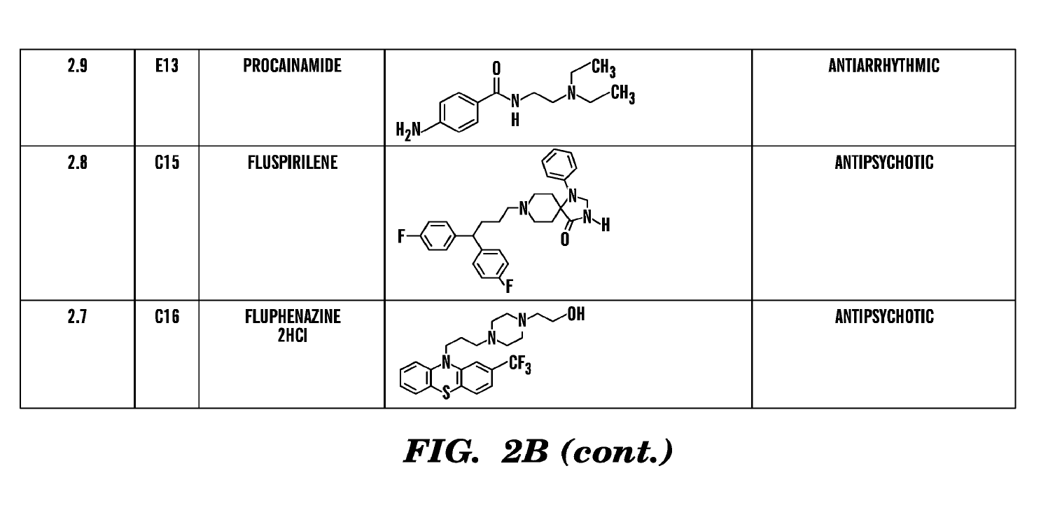

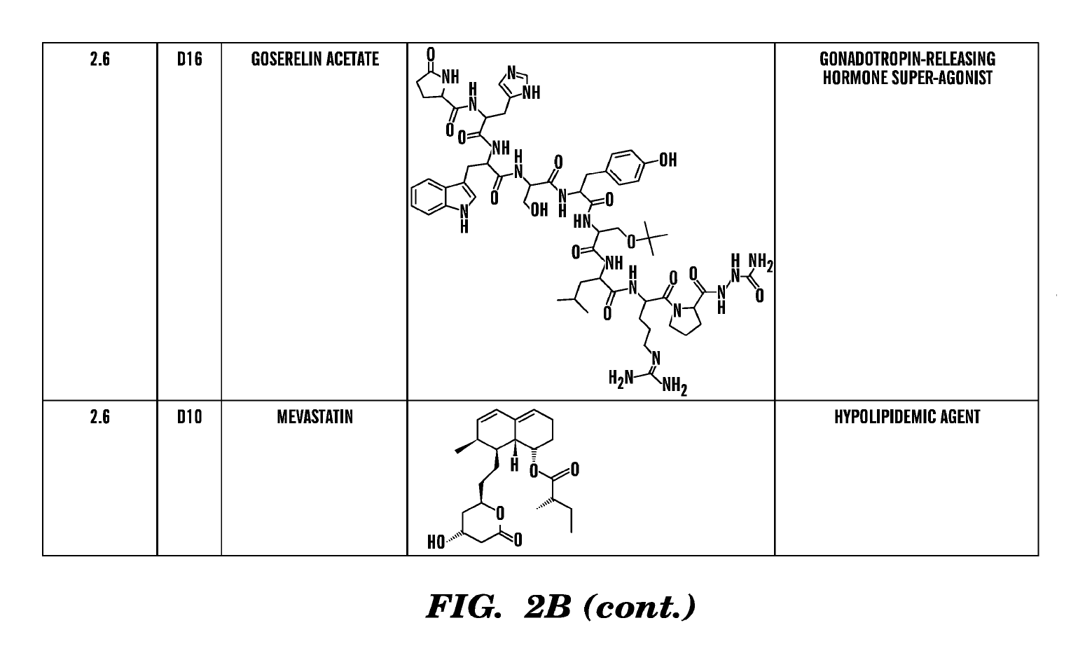

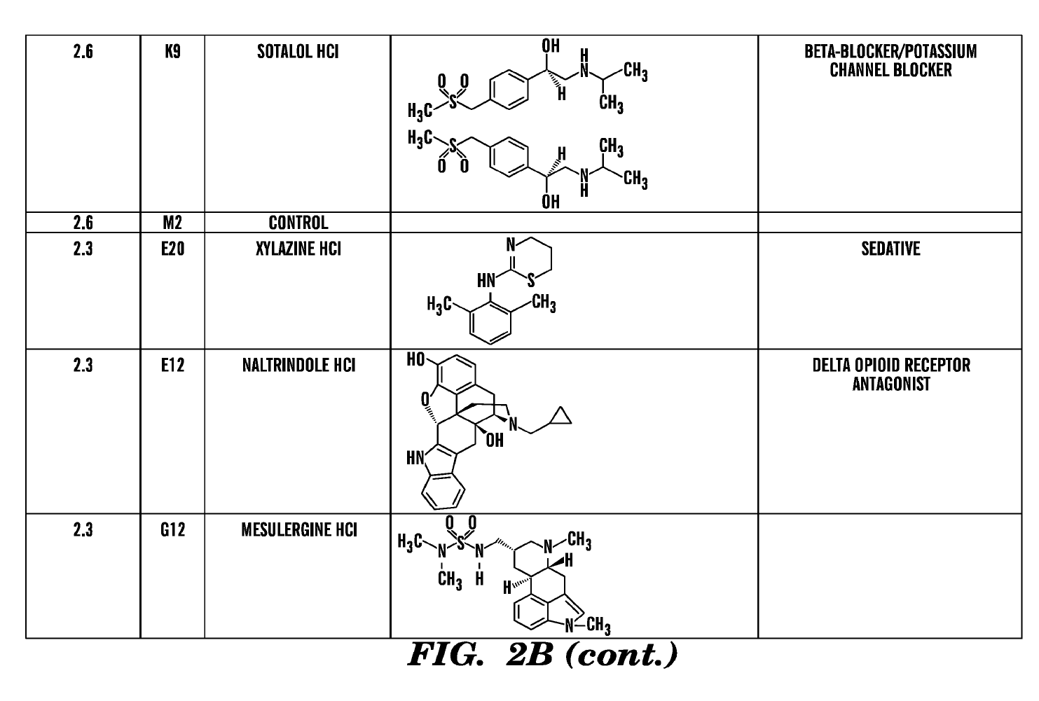

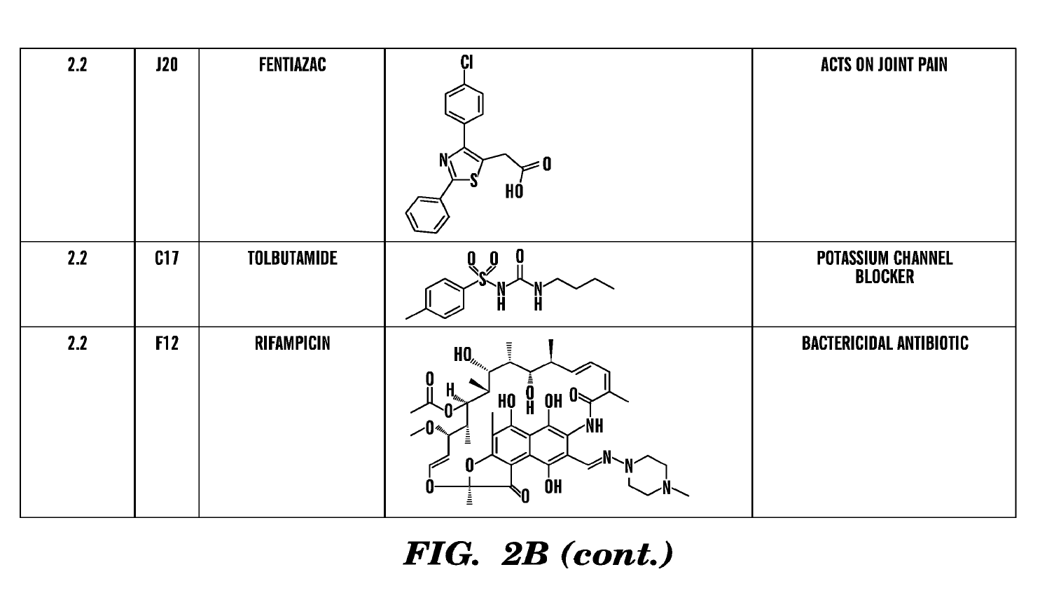

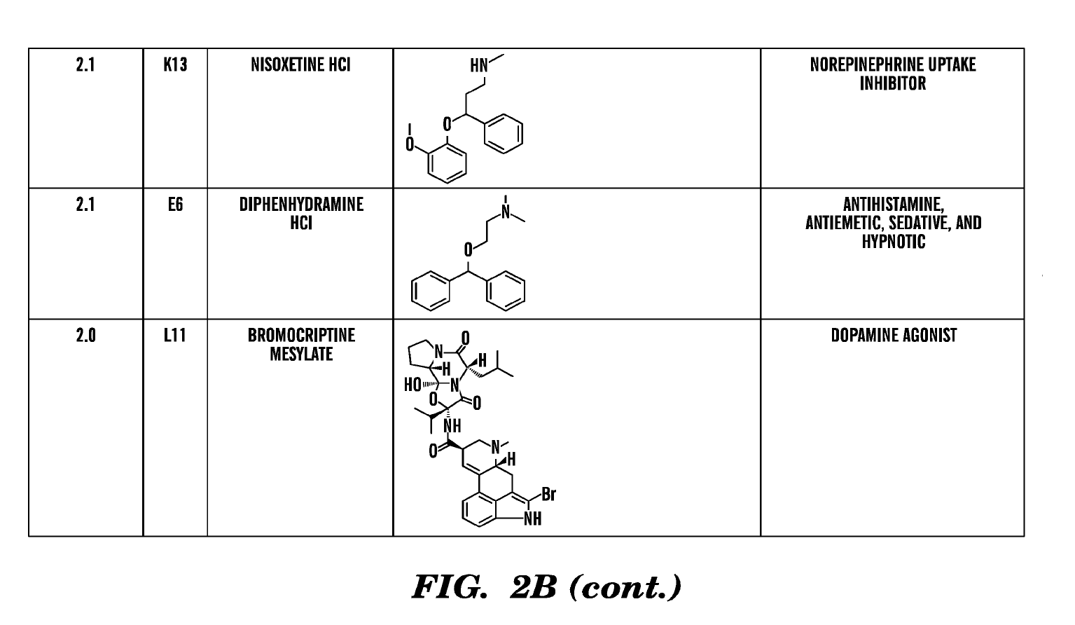

In accordance with various embodiments of the assay described herein, the anabolic efficacies of the plurality of the test compositions are ranked based on the abilities of the test compositions to stimulate muscle growth and/or bone growth of the musculoskeletal cells or precursor cells thereof. The ranking of the anabolic efficacies of the test compositions can be performed by any art-recognized algorithms, which can vary with the methods used to quantify the muscle and/or bone growth. For example, in the analysis of muscle cell proliferation and/or differentiation, images of the cells treated with various test compositions can be analyzed, e.g., with imaging analysis programs such as ImageJ or MATHLAB, for the presence or absence of multi-nucleated muscle cells (e.g., 2 or more nuclei in a cell) formed from mononucleated cells. A fusion or anabolic efficacy index can be determined for each test composition, e.g., based on the number of multi-nucleated cells (e.g., 2 or more nuclei in a cell). In some embodiments, a fusion or anabolic efficacy index can be defined as a ratio of the total number of nuclei involved in cells having at least 2 nuclei, including, e.g., at least 3 nuclei, to the total number of nuclei present in all of the cells (including both mononucleated and multi-nucleated cells). For example, as shown in FIG. 2A, a higher fusion or anabolic efficacy index determined for a test composition indicates that the test composition is capable of inducing a larger fraction of mononucleated cells to fuse together to form multi-nucleated cells. Accordingly, based on the fusion index determined for each test composition, an anabolic rank of the test compositions can be performed based on the abilities of the test compositions to stimulate muscle cell proliferation or differentiation.

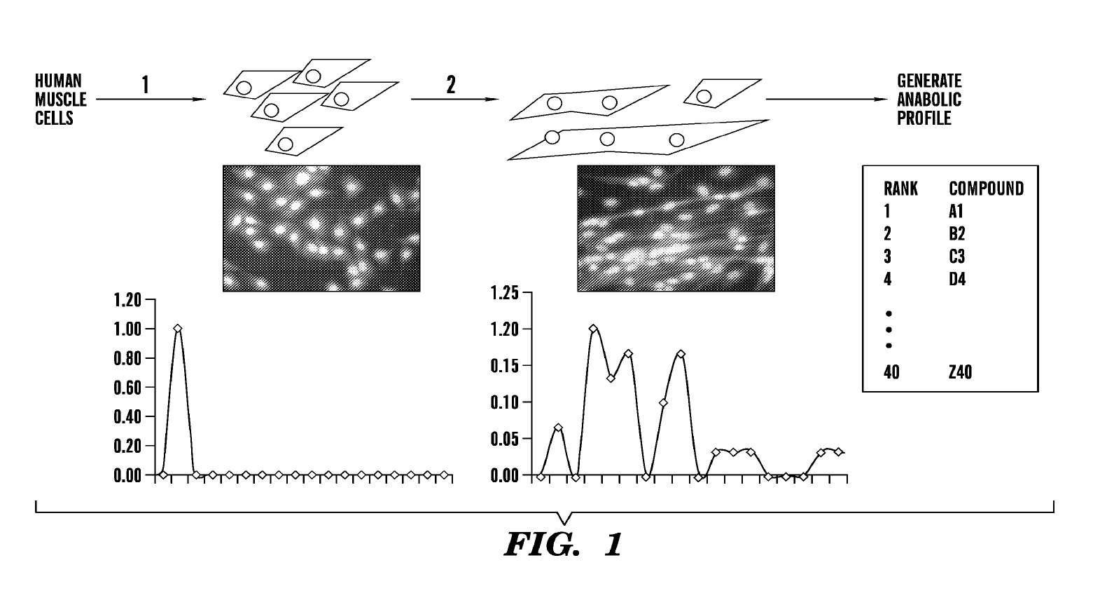

In some embodiments, a fusion or anabolic efficacy index can be defined as the frequency of cells with two or more nuclei per cell, including three or more nucleic per cell. Accordingly, in some embodiments, a fusion distribution can be computed to determine the anabolic rank of the test compositions. By way of example only and not construed to be limiting, a fusion distribution can be determined as follows: Prior to contact with a test composition or an anabolic agent, the number of musculoskeletal or precursor cells thereof in the assay can be determined by any art-recognized method, for example, by high-throughput digital cell imaging with phase contrast microscopy. In addition, the number of nucleus or nuclei within each cell can be determined and/or tabulated. See, e.g., the example fusion distribution corresponding to step 1 (prior to addition of a test composition) of FIG. 1. The initial number of nucleus per cell prior to the anabolic treatment (e.g., prior to the contact of the musculoskeletal cells or precursor cells thereof with a test composition or an anabolic agent) is generally 1.0, i.e., one nucleus per cell. The corresponding nuclear frequency distribution (or fusion distribution) would therefore be 100% for one nucleus/cell, and 0% for two or more nuclei/cell (e.g., 0% for two nuclei/cell, 0% for three nuclei/cell, . . . 0% for ten+nuclei or more per cell. After anabolic treatment, e.g., for a pre-determined period of time, e.g., at least about 6 hours or longer, the process of determining cell number and the number of nuclei per cell can be repeated and displayed as a frequency distribution of 1-10+nuclei/cell. See, e.g., the example fusion distribution corresponding to step 2 (after addition of a test composition) of FIG. 1. The distribution of nuclei per cell can represent the anabolic signature for the tested composition or anabolic agent/composition. Each test composition or anabolic agent/composition can have a unique anabolic signature. Accordingly, in these embodiments, the anabolic rank of the test compositions can be computed based on anabolic treatment of the musculoskeletal cells or precursor cells thereof and computing the frequency of cells with two or more nuclei per cell, which can be compared to the untreated cells (a baseline). In some embodiments, a higher threshold value of a fusion index, e.g., frequency of cells with three or more nuclei per cell, can be used to minimize random variation. Accordingly, in some embodiments, the anabolic rank of the test compositions can be computed as a function of the frequency of cells with three or more nucleic per cell. The highest frequency would be defined as highest rank=1, the second highest frequency as rank=2, etc. In cases where the rank is equivalent, the tie can be broken based on the higher median number of nuclei/cell in the anabolic signature, albeit in this example they have the same frequency of three or more nuclei per cell.

In the analysis of bone cell proliferation and/or differentiation, a ranking of the test compositions with respect to their individual abilities to stimulate bone cell proliferation or differentiation can be performed. In some embodiments, the ranking of the test compositions can be determined based on expression of at least one bone marker (e.g., ALP) in the cells.

In some embodiments where muscle growth and bone growth are both quantified, two separate anabolic profiles or anabolic ranking of the test compositions (e.g., one based on the test composition's ability to induce muscle proliferation and/or differentiation, and another based on the test composition's ability to induce bone proliferation and/or differentiation) can be generated. Alternatively, a combined anabolic profile or anabolic ranking of the test compositions can be generated. For example, a weighted average of the muscle growth and bone growth can be used to generate a combined anabolic profile or anabolic ranking of the test compositions.

In one embodiment, the cell-based assay can comprise (a) contacting muscle cells or muscle precursor cells thereof, which are CD45 negative and CD56 positive and are depleted of fibroblasts, with an array of test compositions each comprising at least one anabolic agent in the presence of growth media; (b) quantifying muscle growth of the cells in response to the test compositions by detecting the number of multi-nucleated cells formed from mononucleated cells, thereby generating a distribution of the number of nuclei per cell distribution; and (c) ranking the test compositions based on their efficacy in promoting muscle growth (cell fusion). The muscle cells or muscle precursor cells (e.g., muscle stem cells) for use in the assay can be isolated from a muscle tissue or biopsy of a subject, or can be provided as banked specimens, and plated in a multi-well culture plate (e.g., a 384 well plate). In some embodiments, it can be desirable to prepare duplicates or triplicates for each test composition. In some embodiments where personal biopsy-derived cells are used, the growth media can comprise autologous serum derived from a blood sample.

### Musculoskeletal Cells or Precursor Cells Thereof and Collection and Preparation of the Same

Musculoskeletal cells or precursor cells thereof used in one or more embodiments of the assays, methods, and systems described herein can generally be collected from one or more subjects, or can include established cell lines. In some embodiments where the assay is used to generate a personalized anabolic profile, the musculoskeletal cells or precursor cells thereof used in the assays, methods, and systems described herein can be collected from a single subject, e.g., who is seeking for personalized guidance on an anabolic treatment. Accordingly, in these embodiments, the musculoskeletal cells or precursor cells thereof used in the assay, method and/or system described herein are subject-specific, and can be obtained or derived from a biological sample of the subject. For example, subject-specific musculoskeletal cells or precursor cells thereof can be obtained or derived from a muscle biopsy and/or a blood sample of the subject.

In some embodiments, the musculoskeletal cells or precursor cells thereof used in one or more embodiments of the assays, methods, and systems described herein can be obtained or derived from cells or tissue specimens representing at least one or more population subgroups, including, e.g., 1, 2, 3, 4, 5, 6, 7, 8, 9, 10, 20, 30, 40, 50, 75, 100, 200, 300, 400, 500 or more population subgroups. The cells or tissue specimens can be obtained from a cell or tissue depository and should be generally well-documented such that they can be stratified to represent different population subgroups based on one or a plurality of features of the population. Examples of the features can include phenotypic features for population stratification including, but not limited to, age or age groups, gender, ethnicity or races, body types, weights, heights, body mass index (BMI), blood types, activity levels (e.g., sedentary lifestyle or work such as a secretary in office vs. active lifestyle or work such as an athlete), chronic or acute diseases (e.g., but not limited to, diabetes, cancer, osteoporosis, HIV infection, infection, musculoskeletal diseases or disorders, metabolic diseases or disorders, and psychophysiological disorders), genetic polymorphisms, diet (e.g., but not limited to, vegetarian, and gluten-free), living habits (e.g., but not limited to, smoking and alcohols), drug resistance, treatment regime such as chemotherapy, drastic/abnormal weight loss, geographical location (e.g., individuals living in the west coast vs. east coast of the United States, or individuals living in the United States vs. in Asian countries) and environmental factors associated therewith, and any combinations thereof. In these embodiments, distinct anabolic profiles for different population subgroups (stratified anabolic profiles) can be generated using one or more embodiments of the assays described herein. If a subject is seeking for guidance on an anabolic treatment, the subject can be matched with one of the stratified population subgroups based on at least one or more features such as phenotypic features as described above and be recommended with an anabolic agent based on its respective ranking in a stratified anabolic profile of the matching population subgroup.

In some embodiments, the musculoskeletal cells or precursor cells thereof for use in the assay, method, system and/or kit described herein can be obtained or derived from a biological sample (e.g., but not limited to, a muscle biopsy and/or blood sample) of subjects or individuals who are determined to suffer from or have a risk for muscle loss and/or bone loss (e.g., a musculoskeletal disease or disorder). Examples of subjects or individuals who are at risk for a musculoskeletal disease or disorder include, but are not limited to, athletes, aging individuals, individuals having a chronic disease or disorder (e.g., but not limited to, cancer, chronic obstructive pulmonary disease (COPD), chronic kidney disease (CKD), chronic liver failure (CLF), and chronic infections), individuals suffering from malnutrition, or any combinations thereof. Examples of a musculoskeletal disease or disorder include, but are not limited to, muscle loss, muscle wasting, muscle wasting associated with HIV infection, muscle wasting in cancer survivors, cachexia, muscular dystrophy, osteopenia, osteoporosis, sarcopenia, an age-related musculoskeletal disease or disorder, or a musculoskeletal disease or disorder associated with anabolic resistance, a musculoskeletal disease or disorder associated with excessive weight loss, or any combinations thereof.

In some embodiments, the musculoskeletal cells or precursor cells thereof for use in the assay, method, system and/or kit described herein can be obtained or derived from a biological sample of subjects or individuals who have previously shown non-responsiveness or resistance to at least one or more anabolic agents.

In some embodiments, the musculoskeletal cells or precursor cells thereof for use in the assay, method, system and/or kit described herein can be obtained or derived from a biological sample of subjects or individuals who are seeking to maintain and/or enhance muscle and/or bone health.

In some embodiments, the musculoskeletal cells or precursor cells thereof for use in the assay, method, system and/or kit described herein can be obtained or derived from a biological sample of subjects or individuals who are normal healthy subjects without any known bone and/or muscle loss.

As used herein, the term “musculoskeletal cells” generally refers to cells associated with muscle, bone, cartilage, connective tissue or any combinations thereof. In some embodiments, musculoskeletal cells used in the assays, methods, systems and/or kits described herein can include muscle cells. Depending on the type of musculoskeletal disease or disorder, and/or the location of symptoms which can include pain or weakness, the musculoskeletal cells can be obtained or derived from a muscle biopsy of any tissue (e.g., heart tissue, leg muscles such as quandriceps, and arm muscles such as biceps, shoulder muscles such as deltoid) in a subject.

As used herein, the term “precursor cells thereof” generally refers to precursor cells or stem cells that can be differentiated into musculoskeletal cells, including, but are not limited to, muscle precursor cells, bone precursor cells, and/or any cells that can be differentiated into muscle cells or bone cells. Muscle precursor cells can encompass any cells that differentiate towards the myogenic lineage upon treatment with at least one known myoblast-promoting (e.g., but not limited to, bFGF, aFGF, IGF-1, and NGF). In one embodiment, muscle precursor cells can include mononucleated myoblasts. Bone precursor cells can encompass cells that differentiate towards the osteoblast lineage upon treatment with at least one known osteoblast-promoting agents (e.g., but not limited to type I collagen, fibrinogen, fibrin, fibrinogen, osteocalcin, osteonectin, TGF-β, Vitamin D3, basic fibroblast growth factor, or bone morphogenic protein 2). In one embodiment, bone precursor cells can include osteoprogenitor cells or preosteoblasts. Without limitations, muscle precursor cells and/or bone precursor cells can be obtained or derived from a muscle biopsy and/or from peripheral blood progenitor cells or stem cells, e.g., using induced pluripotent cell technology known in the art. Accordingly, in some embodiments, a blood sample can be collected from a subject as a source of musculoskeletal cells or precursor cells thereof in one or more embodiments of the assays, methods, and/or systems described herein.

In some embodiments, the musculoskeletal cells or precursor cells thereof for use in the assays, methods, kits, and/or systems described herein can be pre-selected for CD45 negative and/or CD56-positive. Without wishing to be bound by theory, the cells that are CD45-negative and CD56-positive can be muscle-derived stem cells. Accordingly, in some embodiments, muscle-derived stem cells or muscle precursor stem cells can be used in the assays, methods, kits and/or systems described herein. Methods for isolating muscle-derived stem cells from a muscle biopsy are known in the art. See, e.g., Sharifiaghdas et al., Urol J. 2011 Winter; 8(1):54-9 or Example 4 for example methods to prepare human muscle-derived stem cells or muscle precursor stem cells) from a muscle biopsy. For example, CD45-positive musculoskeletal cells or precursor cells thereof can be first removed from the population of cells using magnetic beads coated with CD45-binding molecules. The remaining CD45-negative musculoskeletal cells or precursor cells thereof can be further positively selected for CD56-positive cells, e.g., using magnetic beads coated with CD56-binding molecules, which are considered as muscle-derived stem cells or muscle precursor stem cells.

In some embodiments, the musculoskeletal cells or precursor cells thereof for use in the assays, methods, kits, and/or systems described herein exclude fibroblasts. The fibroblasts can be removed from the population of musculoskeletal or precursor cells thereof using any methods known in the art. For example, fibroblasts can be removed from the population of cells using magnetic beads and/or by performing a fibroblast plate adherence depletion. An exemplary fibroblast plate adherence depletion assay includes plating the cell population to a non-ECM-coated plate where fibroblasts can attach to the plate in the absence of the ECM, while other cells cannot. Thus, the non-attached musculoskeletal or precursor cells thereof can be collected, e.g., by centrifugation.

Collections of a biopsy or a blood sample for at least one analysis performed in the assays and/or systems described herein are well known to those skilled in the art. The sample can be obtained by removing a sample of cells from a subject, but can also be accomplished by using previously isolated cells (e.g. isolated by another person). In addition, the cells can be freshly collected or a previously collected sample.

In some embodiments, the sample for use in one or more embodiments of the assay or system described herein can be a frozen sample, e.g., a frozen tissue or blood sample. The frozen sample can be thawed before employing assays and systems described herein. After thawing, a frozen sample can be centrifuged before being subjected to assays and systems described herein.

To collect a muscle tissue, by way of example only, a patient's muscle biopsy, e.g., a percutaneous muscle biopsy, can be performed by a trained medical personnel. To collect a blood sample, by way of example only, the patient's blood can be drawn by trained medical personnel directly into anti-coagulants such as citrate, EDTA PGE, and theophylline. The whole blood can be separated into the plasma portion, the cells, and platelets portion by refrigerated centrifugation at 3500 g for 2 minutes. After centrifugation, the supernatant is the plasma and the pellet is RBC. Since platelets have a tendency to adhere to glass, it is preferred that the collection tube be siliconized. Another method of isolating red blood cells (RBCs) is described in Best, C A et al., 2003, J. Lipid Research, 44:612-620. In order to collect peripheral blood stem cell from a blood sample of a subject, prior to the collection, the subject can be given a medication to promote the growth and release of stem cells from the bone into the blood. The stem cells are then collected using a special machine called a cell separator.

### Cell Culture and Substrate Materials Employed in the Assays, Systems or Kits Described Herein

In order to determine different anabolic responses (e.g., muscle growth and/or bone growth) of the musculoskeletal cells or precursor cells thereof to one or more test compositions, in some embodiments, the cells can be cultured and/or maintained in separate conditions optimized for promoting muscle cell growth/proliferation and/or bone cell growth/proliferation, prior to and/or during the contact with the test compositions. For example, the musculoskeletal cells or precursor cells thereof can be cultured in a growth medium optimized to mimic the original microenvironment of a subject or pooled subjects within a population subgroup from whom the cells were collected. Without wishing to be bound by theory, serum from individual subjects can have different effects on muscle growth and/or bone growth. By way of example only, it has been previously shown in mouse models that transfer of serum from younger subjects to older subjects can accelerate muscle regeneration and conversely, serum from older subjects to younger subjects can inhibit muscle growth. Accordingly, in some embodiments, serum collected from a blood sample from a subject for personalized biopsy or from pooled subjects within a population subgroup can be added to the growth medium to re-create the microenvironment of the host. The serum can be added to the growth medium in any amount. For example, the serum can be added to reach at least about 1% or higher by volume in the growth medium, including, e.g., but not limited to, at least about 5%, at least about 10%, at least about 20%, at least about 30% or more, by volume in the growth medium. In one embodiment, the growth medium has about 10% (by volume) of serum.

While not necessary, the growth medium in which the musculoskeletal cells or precursor cells thereof are cultured prior to or after addition of the test compositions described herein can have the same or different composition.

In some embodiments, the cells can be additionally or alternatively cultured and/or maintained in a separate substrate material specific to muscle differentiation/growth and bone differentiation/growth, prior to and/or during the contact with the test compositions.

The term “substrate material” as used herein refers to a material that facilitates muscle and/or bone growth and/or differentiation. The substrate material can comprise a synthetic material, a natural material, or both. In some embodiments, the substrate material can comprise at least one biocompatible material that facilitates, permits, or enhances deposition of new tissue matrix (e.g., muscle- or bone-related matrices). In some embodiments, the substrate material can comprise mineralized materials, such as calcium sulfate or calcium phosphate, a biopolymer, a metal, allograft muscle tissue, autograft muscle tissue, demineralized bone matrix, or any combinations thereof.

In one embodiment, the substrate material includes extracellular matrix. Extracellular matrix (ECM) for culture of muscle cells or precursor cells thereof obtained from a subject can include collagen, non-collagenous glycoproteins, proteoglycans, or any combinations thereof. The extracellular matrix can include any art-recognized molecules such as growth factor (e.g., fibroblast growth factor) to mimic the native environment of a musculoskeletal tissue for promotion of muscle or bone differentiation and/or growth. See, e.g., Kjaer M. (2004) Physiol. Rev. 84: 649, for typical ECM components in a muscle.

The substrate material can be planar in shape or form a scaffold. In one embodiment, the substrate material employed in the assays, systems or kits for culturing musculoskeletal cells or precursor cells thereof forms a scaffold. The term “scaffold” as used herein generally refers to a 3-dimensional supporting structure that promotes muscle and/or bone growth. Non-limiting examples of a scaffold includes a gel, a solid, a matrix, a hydrogel, a sponge, a mesh, a membrane and any combinations thereof. In some embodiments, the scaffold substrate can be porous.

In one embodiment, the substrate material for use in the assays, systems and kits described herein includes an ECM scaffold, e.g., a scaffold comprising at least one type of extracellular matrix molecules, fibers, and/or fibrils, including at least two, at least three, at least four or more extracellular matrix molecules, fibers, and/or fibrils.

In some embodiments, the substrate material for use in the assays, systems and kits described herein can include Matrigel and/or Engelbreth-Holm-Swarm sarcoma ECM.

Muscle Cell-Specific Culture Conditions:

The stiffness of the substrate material can also affect anabolic responses of cells to a test composition. Thus, it is desirable to have a substrate material with a defined stiffness optimal to promote muscle and/or bone growth and/or differentiation. For example, to determine muscle growth-responses of musculoskeletal cells or precursor cells thereof to a test composition, in some embodiments, the cells can be cultured and/or maintained, prior to and/or during the contact with a test composition, in a substrate material with a defined stiffness optimal to promote muscle cell proliferation and/or differentiation, e.g., a defined stiffness of about 5 kPa to about 50 kPa, or about 10 kPa to about 20 kPa. In some embodiments, the stiffness of the substrate material for stimulation of muscle cell proliferation and/or differentiation can vary with the microenvironment of the tissue from which the musculoskeletal cells or precursor cells thereof were collected or derived.

In some embodiments, the musculoskeletal or precursor cells thereof (e.g., but not limited to, muscle precursor stem cells) can be cultured in a growth medium adapted to induce cell differentiation. For example, the musculoskeletal or precursor cells thereof (e.g., but not limited to, muscle precursor stem cells) can be cultured in a growth medium adapted to induce cell fusion to form multi-nucleated muscle cells, and/or to induce differentiation of non-differentiated stem cells or muscle precursor cells to form muscle cells. In some embodiments, the growth medium can comprise a serum purified or collected from a blood sample of a specific subject or pooled subjects within a population subgroup as described above.

Bone Cell-Specific Culture Conditions:

Similarly, to determine bone growth-responses of musculoskeletal cells or precursor cells thereof to a test composition, in some embodiments, the cells can be cultured and/or maintained, prior to and/or during the contact with a test composition, in a substrate material with a defined stiffness optimal to promote bone cell proliferation and/or differentiation, e.g., a defined stiffness of about 10 kPa to about 150 kPa, or about 20 kPa to about 100 kPa. In some embodiments, the stiffness of the substrate material for stimulation of bone cell proliferation and/or differentiation can vary with the microenvironment of the tissue from which the musculoskeletal cells or precursor cells thereof were collected or derived.

In some embodiments, the stiffness, composition and/or structure of the substrate material can be adjusted such that the substrate material is osteoconductive, osteoinductive, osteogenic, or any combinations thereof. For example, while not necessary, in some embodiments, the substrate material for stimulation of bone growth and/or differentiation can include an osteoconductive, osteoinductive, osteogenic agent, e.g., bone morphogenetic protein-2 (BMP-2), any derivatives thereof, or any art-recognized bone-inducing agents such as the agents disclosed in U.S. Pat. No. 7,897,588, the content of which is incorporated by reference.

As used herein, the term “osteoconductive” generally refers to the ability of a material or agent to facilitate the migration of osteogenic cells within a substrate material. In some embodiments, the porosity of the substrate material can affect its osteoconductivity. As used herein, the term “osteoinductive” generally refers to the ability to induce non-differentiated stem cells or osteoprogenitor cells (osteoblasts), which is a component of osseous (bone) tissue, to differentiate into osteoblasts. The simplest test of osteoinductivity is the ability to induce the formation of bone in tissue locations such as muscle, which do not normally form bone (ectopic bone growth). It is generally understood that a substrate material can be made osteoinductive by adding growth factors such as rhBMP-2 (recombinant human bone morphogenic protein-2) to it. The mineralization and the addition of growth factors can affect the osteoinductivity of the substrate material. As used herein, the term “osteogenic” generally refers to the ability of forming new bone cells or tissue. Osteogenesis is the process of laying down new bone material using osteoblasts. Osteoblasts build bone by producing osteoid to form an osteoid matrix, which is composed mainly of Type I collagen. Osseous tissue comprises the osteoid matrix and minerals (mostly with calcium phosphate) that form the chemical arrangement termed calcium hydroxyapatite. Osteoblasts are typically responsible for mineralization of the osteoid matrix to form osseous tissue. Without wishing to be bound by a theory, the osteoconductivity and osteoinductivity of the substrate material can have an impact on osteogenesis.

In some embodiments, the musculoskeletal cells or precursor cells thereof (e.g., but not limited to, muscle or bone precursor stem cells) can be cultured in a growth medium adapted to induce cell differentiation. For example, the musculoskeletal or precursor cells thereof (e.g., but not limited to, muscle or bone precursor stem cells) can be cultured in a growth medium adapted to differentiate muscle cells, bone precursor cells, non-differentiated stem cells to become bone cells. In some embodiments, the growth medium can comprise a serum purified or collected from a blood sample of a specific subject or pooled subjects within a population subgroup as described above.

While not necessary, in some embodiments, the musculoskeletal cells or precursor cells thereof can be cultured in the presence of a bone formation-inducing agent. Examples of a bone formation-inducing agent can include, but are not limited to, bone morphogenic factor (BMP) (e.g., BMP-1, BMP-2, BMP-3, BMP-4, BMP-5 and BMP-6), transforming growth factor (TGF), insulin-like growth factor (IGF), basic fibroblast growth factor (bFBF), osteogenic protein (OP) (e.g., OP-1, OP-2 and OP-3), osteogenic factors, osteoconductive factors, osteoinductive factors, and any combinations thereof. In one embodiment, the bone formation-inducing agent can include bone morphogenetic protein-2 (BMP-2).

In vitro culture of muscle cells and precursor cells thereof for muscle cell proliferation/differentiation or bone cell proliferation/differentiation are known in the art. A skilled artisan can optimize muscle cell-specific culture conditions and bone cell-specific culture conditions for musculoskeletal cells or precursor cells thereof.

In some embodiments, rather than harvesting muscle cells from a subject's muscle biopsy for use in the assays, systems, and/or kits described herein, stem cells collected from a subject (e.g., a subject's blood sample) can be used in the assays, methods, systems and/or kits described herein and be cultured in an appropriate condition suitable for muscle cell differentiation or bone cell differentiation. Methods for generation of muscle cells from stem cells are known in the art. See, e.g., Salani S et al. “Generation of skeletal muscle cells from embryonic and induced pluripotent stem cells as an in vitro model and for therapy of muscular dystrophies” (2012) J Cell Mol Med. 16:1353; Yoshida Y and Yamanaka S. “Recent Stem Cell Advances: Induced Pluripotent Stem Cells for Disease Modeling and Stem Cell-Based Regeneration Circulation.” (2010) Circulation. 122: 80; and Bilousova G. et al., “Osteoblasts derived from induced pluripotent stem cells form calcified structures in scaffolds both in vitro and in vivo.” (2011) Stem Cells. 29: 206-16.

### Muscle Growth of Musculoskeletal Cells or Precursor Cells Thereof (e.g., Mononucleated Muscle Cells, Muscle Precursor Cells Thereof Including Blood-Derived Cells) and Exemplary Methods/Analyses to Quantify Muscle Growth

In one or more embodiments of the assays described herein, the musculoskeletal cells or precursor cells thereof after contact with a plurality of test compositions can be subjected to at least one or more analyses to quantify muscle growth of the cells in response to the test compositions. In one embodiment, the musculoskeletal cells or precursor cells thereof include mononucleated muscle cells. The mononucleated muscle cells, myoblasts, are uniquely different from other cells in the body in a number of ways: 1) myoblasts naturally differentiate to form muscle tubules capable of muscle contraction, 2) when myoblasts fuse to form myotubes, these cells become post mitotic (stop dividing) with maturation, and 3) as myotubes, the cells express large amounts of protein which is produced in the cells due to multinucleation. Accordingly, as used herein, the term “muscle growth,” in some embodiments, refers to an increase in differentiation of mononucleated muscle cells, myoblasts, e.g., collected from a muscle biopsy, into myotubes, or multi-nucleated muscle cells. For example, the term “muscle growth” refers to an increase in the number of mononucleated muscle cells, myoblasts, differentiated into multi-nucleated muscle cells by at least about 10%, at least about 20%, at least about 30%, at least about 40%, at least about 50%, at least about 60%, at least about 70%, at least about 80%, at least about 90%, at least about 95%, or more, as compared to the number of myoblasts in the absence of a test composition described herein. In other embodiments, the term “muscle growth” refers to an increase in proliferation of myoblasts. For example, the proliferation of myoblasts is increased by at least about 10%, at least about 20%, at least about 30%, at least about 40%, at least about 50%, at least about 60%, at least about 70%, at least about 80%, at least about 90%, at least about 95%, or more, as compared to the number of myoblasts in the absence of a test composition described herein. In some embodiments, the term “muscle growth” refers to both an increase in proliferation of myoblasts (e.g., by at least about 10%) and an increase in differentiation of the mononucleated myoblasts into multi-nucleated muscle cells (e.g., by at least about 10% in cell number), as compared to the number of myoblasts in the absence of a test composition described herein.

Any art-recognized methods and/or analyses to quantify muscle growth of myoblasts or blood-derived muscle cells in vitro can be employed in one or more embodiments of the assay described herein. For example, the muscle growth of myoblasts in vitro can be determined by examining the formation of multinucleated muscle cells (myotubes) by fusion of mononucleated muscle cells (myoblasts), e.g., by microscopy and/or histological methods.

Alternatively, the muscle growth of myoblasts in vitro can be determined by detecting one or more proteins and/or genes involved in muscle cell differentiation. Genes, such as muscle creatine kinase, troponin, caveolin 3, α-actin, and myosin, are reported to be expressed predominantly in the skeletal muscles. A family of transcription factors specifically expressed in the muscles, including myoD, myogenin, myf-5, and MRF-4/herculin/myf-6, have been cloned. These factors are phosphorylated nuclear proteins containing a helix-loop-helix (bHLH) motif, as required for both dimerization and DNA binding, and are believed to be determinants of the cell-specific differentiation program (Olson and Klein (1994), Genes & Dev. 8:1-8). When one of these factors is introduced into non-myogenic cells, differentiation into mature muscle cells is initiated (Weintraub et al. (1991), Science 251:761-766). The myoD family, a group of transcription factors, has been found to direct muscle formation, inhibit proliferation, activate differentiation and induce a contractile phenotype. While myoD and myf-5 are expressed within the proliferating myoblasts, myogenin and MRF-4 are not expressed until the myoblasts withdraw from the cell cycle in response to mitogen withdrawal. Based on these findings, it was demonstrated that myogenin and MRF-4 activate and maintain the expression of muscle-specific genes (Emerson (1993), Curr. Opin. Genet. Dev. 3:265-274), while myoD and myf-5 are thought to play a role in the proliferation of myoblasts. Other cell-cycle regulatory proteins, such as RB (Shiio et al. (1996), Oncogene 12:1837-1845, Wang et al. (1997), Cancer Research 57:351-354), p21 (Guo et al. (1995), Mol. Cell Biol. 15:3823-3829), cyclin D, cdk2, cdk4 (Kiess et al. (1995), Oncogene 10:159-166) and tumor suppressor gene p53 (Soddu et al. (1996), J. Cell Biol. 134:193-204) are involved in the muscle cell differentiation program. Recently, caveolin 3 (Song et al. (1996), J. Cell Biol. 271:15160-15165), α-dystroglycan (Kostrominova and Tanzer (1995), J. Cell Biochem. 58:527-534) and DNA methyltransferases (Takagi et al. (1995), Eur. J. Biochem. 231:282-291) have been shown to play positive roles in myogenic differentiation. Methods for detecting expression of proteins and/or genes in cells are known in the art and can be used herein to detect the presence or absence of any protein and/or genes that induce myoblast proliferation and/or differentiation. Other proteins and/or genes involved in myocyte differentiation, e.g., the ones discussed in U.S. Pat. No. 6,670,450, can also be employed to quantify muscle growth in vitro.

### Bone Growth of Musculoskeletal Cells or Precursor Cells Thereof (e.g., Muscle Cells, or Bone Precursor Cells Thereof Including Blood-Derived Cells) and Exemplary Methods/Analyses to Quantify Bone Growth

In one or more embodiments of the assay described herein, the musculoskeletal cells or precursor cells thereof after contact with a plurality of test compositions can be subjected to at least one or more analyses to quantify in vitro bone growth of the cells in response to the test compositions. As used herein, the term “bone growth,” in some embodiments, refers to an increase in differentiation of muscle cells or precursor cells thereof into bone or osteoblast phenotype cells. For example, the term “bone growth” refers to an increase in the number of muscle cells or bone precursor cells differentiated into bone or osteoblast phenotype cells by at least about 10%, at least about 20%, at least about 30%, at least about 40%, at least about 50%, at least about 60%, at least about 70%, at least about 80%, at least about 90%, at least about 95%, or more, as compared to the cells in the absence of a test composition described herein. In some embodiments, the term “bone growth” can further encompass an increase in proliferation of osteoblast phenotype cells. For example, the proliferation of osteoblast phenotype cells can be increased by at least about 10%, at least about 20%, at least about 30%, at least about 40%, at least about 50%, at least about 60%, at least about 70%, at least about 80%, at least about 90%, at least about 95%, or more, as compared to the cells in the absence of a test composition described herein. In some embodiments, the term “bone growth” refers to both an increase in differentiation of the muscle cells and/or bone precursor cells into bone or osteoblast phenotype cells (e.g., by at least about 10% in cell number) and an increase in proliferation of the osteoblast phenotype cells (e.g., by at least about 10%), as compared to the cells in the absence of a test composition described herein.

As used herein, the term “osteoblast phenotype cells” refers to cells displaying at least one phenotype or characteristic associated with osteoblasts. The osteoblast phenotype cells including terminally or non-terminally differentiated cells can be derived from bone precursor cells thereof including stem cells. Examples of osteoblast-associated phenotype or characteristic include but are not limited to, cuboidal morphology of the cells, production of alkaline phosphatase (ALP), production of type I collagen production, production of osteocalcine, production of mineralized extracellular matrix, expression of one or more specific marker transcripts, such as but not limited to, AP-1 family members, Runx2, Fra-2, alkaline phosphatase, osteocalcin, β-catenin, CCAAT/enhancer binding protein (C/EBP), and ATF4, or any combinations thereof. In some embodiments, the osteoblast phenotype cells can advance in differentiation all the way to terminal differentiation (e.g., with the production of a mineralized extracellular matrix), and these cells are termed as “terminally differentiated osteoblasts” herein.

Accordingly, in order to quantify the bone growth in musculoskeletal cells or precursor cells thereof after contact with a test composition, methods for determining the activity of alkaline phosphatase (ALP), the expression of type I collagen, and osteocalcine, and any mineralization of the extracellular matrix can be employed in one or more embodiments of the assay or systems described herein.

In one embodiment, the musculoskeletal cells or precursor cells thereof after contact with a test composition can be subjected to an analysis of ALP level or activity expressed by the osteoblast phenotype cells. For example, alkaline phosphatase (ALP) activity can be determined by using para-nitrophenol phosphatase as a substrate, using the technique described by L. Lecoeur and J. P. Ouhayoun, Biomaterials 18, 989-993 (1997). The quantity of para-nitrophenol formed upon hydrolysis of the substrate can be determined by measuring the absorbance at 410 nm, which is converted into nanomoles of enzyme using a calibration curve established on the basis of known concentrations of para-nitrophenol. A test can also be performed for detecting in situ the activity of alkaline phosphatase on cell cultures fixed with formaldehyde, using a kit for semi-quantitative histochemical detection of alkaline phosphatase (kits sold by Sigma Chemical Co., reference 86R). Alkaline phosphatase activity can then be visualized on the cell mat by a reddish color. Alternatively, ALP level can be determined by staining as described in the Examples.

In some embodiments, the musculoskeletal cells or precursor cells thereof after contact with a test composition can be subjected to an analysis of collagen production, osteocalcine expression, or both. For example, the presence of collagens (e.g., of type I and of type II) or osteocalcine can be determined by an immunoassay, e.g., antibody-based assay and immunostaining, and/or gene expression measurements (e.g., PCR, and/or quantitative PCR).

In some embodiments, the musculoskeletal cells or precursor cells thereof after contact with a test composition can be subjected to an analysis of possible mineralization of the extracellular matrix. For example, mineralization of the extracellular matrix can be detected by using the von Kossa stain test implemented using the technique described by Cheng et al., Endocrinology 134: 277-285 (1994).

In some embodiments, the musculoskeletal cells or precursor cells thereof after contact with a test composition can be subjected to an analysis to determine expression of at least one art-recognized marker for osteoblast differentiation, e.g., but not limited to, AP-1 family members, Runx2, Fra-2, alkaline phosphatase, osteocalcin, β-catenin, CCAAT/enhancer binding protein (C/EBP), and ATF4, or to determine a gene expression profile associated with osteoblast differentiation. Additional bone markers, e.g., as described in U.S. Pat. App. No. US 2004/0101818, can be utilized in one or more embodiments of the assay or system described herein to quantify the presence or absence of bone growth of the musculoskeletal cells or precursor cells thereof in response to a test composition. For example, the presence of bone growth of the musculoskeletal cells or precursor cells thereof in response to a test composition can be indicated by an increase in expression of at least one art-recognized marker for osteoblast differentiation, e.g., but not limited to, AP-1 family members, Runx2, Fra-2, alkaline phosphatase, osteocalcin, β-catenin, CCAAT/enhancer binding protein (C/EBP), and ATF4, by at least about 10%, at least about 20%, at least about 30%, at least about 40%, at least about 50%, at least about 60%, at least about 70%, at least about 80%, at least about 90%, at least about 95% or more, as compared to the cells in the absence of the test composition.

Exemplary Methods of Determining Expression Level of Markers for Differentiated Muscle Cells and/or Bone Cells

Measuring Protein Expression:

Various methods known in the art can be used to determine expression of markers or genes specific for differentiated muscle cells and bone cells as described earlier. In some embodiments, the protein expression level of markers or genes specific for differentiated muscle cells or bone cells as described earlier can be measured to quantify muscle growth or bone growth, respectively, of musculoskeletal cells or precursor cells thereof in response to a test composition. By way of example only, the levels of protein markers can be measured by contacting a test sample with an antibody-based binding moiety that specifically binds to at least one of the markers specific for differentiated muscle or bone cells, or to a fragment thereof. Formation of the antibody-protein complex is then detected by a variety of methods known in the art.

The term “antibody-based binding moiety” or “antibody” can include immunoglobulin molecules and immunologically active determinants of immunoglobulin molecules, e.g., molecules that contain an antigen binding site which specifically binds (immunoreacts with) to the markers specific for differentiated muscle or bone cells. The term “antibody-based binding moiety” is intended to include whole antibodies, e.g., of any isotype (IgG, IgA, IgM, IgE, etc), and includes fragments thereof which are also specifically reactive with the markers described herein specific for differentiated muscle or bone cells. Antibodies can be fragmented using conventional techniques. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, dAbs and single chain antibodies (scFv) containing a VL and VH domain joined by a peptide linker. The scFv's can be covalently or non-covalently linked to form antibodies having two or more binding sites. Thus, “antibody-base binding moiety” includes polyclonal, monoclonal, or other purified preparations of antibodies and recombinant antibodies. The term “antibody-base binding moiety” is further intended to include humanized antibodies, bispecific antibodies, and chimeric molecules having at least one antigen binding determinant derived from an antibody molecule. In some embodiments, the antibody-based binding moiety can be detectably labeled.

“Labeled antibody”, as used herein, includes antibodies that are labeled by a detectable means and include, but are not limited to, antibodies that are enzymatically, radioactively, fluorescently, and chemiluminescently labeled. Antibodies can also be labeled with a detectable tag, such as c-Myc, HA, VSV-G, HSV, FLAG, V5, or HIS. The detection and quantification of the marker proteins in test samples correlate to the intensity of the signal emitted from the detectably labeled antibody.

In some embodiments, the antibody-based binding moiety can be detectably labeled by linking the antibody to an enzyme. The enzyme, in turn, when exposed to its substrate, will react with the substrate in such a manner as to produce a chemical moiety which can be detected, for example, by spectrophotometric, fluorometric or by visual means. Enzymes which can be used to detectably label the antibodies against the marker protein specific for differentiated muscle or bone cells can include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

Detection can also be accomplished using any of a variety of other immunoassays. For example, by radioactively labeling an antibody, it is possible to detect the antibody through the use of radioimmune assays. The radioactive isotope can be detected by such means as the use of a gamma counter or a scintillation counter or by autoradiography. Isotopes which are particularly useful for the purpose of the present invention are ³H, ¹³¹I, ³⁵S, ¹⁴C, and ¹²⁵I.

It is also possible to label an antibody with a fluorescent compound. When the fluorescently labeled antibody is exposed to light of the proper wavelength, its presence can then be detected due to fluorescence. Examples of the most commonly used fluorescent labeling compounds include, but not limited to, CYE dyes, fluorescein isothiocyanate, rhodamine, phycoerytherin, phycocyanin, allophycocyanin, o-phthaldehyde and fluorescamine.

An antibody can also be detectably labeled using fluorescence emitting metals such as ¹⁵²Eu, or others of the lanthanide series. These metals can be attached to the antibody using such metal chelating groups as diethylenetriaminepentaacetic acid (DTPA) or ethylenediaminetetraacetic acid (EDTA).

An antibody also can be detectably labeled by coupling it to a chemiluminescent compound. The presence of the chemiluminescent-antibody is then determined by detecting the presence of luminescence that arises during the course of a chemical reaction. Examples of chemiluminescent labeling compounds can include, but not limited to, luminol, luciferin, isoluminol, theromatic acridinium ester, imidazole, acridinium salt and oxalate ester.

Without limitations, levels of the marker specific for differentiated muscle or bone cells can be detected by immunoassays, such as enzyme linked immunoabsorbant assay (ELISA), radioimmunoassay (RIA), Immunoradiometric assay (IRMA), Western blotting, immunocytochemistry or immunohistochemistry, each of which are described in more detail below. In some embodiments, immunoassays such as ELISA or RIA can be used for determining expression levels of the markes specific for muscle and/or bone cells. Antibody arrays or protein chips can also be employed, see for example U.S. Patent Application Nos. 2003/0013208A1; 2002/0155493A1; 2003/0017515 and U.S. Pat. Nos. 6,329,209; 6,365,418, which are herein incorporated by reference in their entirety. Commercially available antibodies and/or immunoassays (such as ELISA) for detecting specific markers for differentiated muscle or bone cells can be used in the assays and/or systems described herein.

Immunoassays:

The most common enzyme immunoassay is the “Enzyme-Linked Immunosorbent Assay (ELISA).” ELISA is a technique for detecting and measuring the concentration of an antigen using a labeled (e.g. enzyme linked) form of the antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem., 22:895-904.

In a “sandwich ELISA”, an antibody (e.g. anti-enzyme) is linked to a solid phase (i.e. a microtiter plate) and exposed to a biological sample containing antigen (e.g. enzyme). The solid phase is then washed to remove unbound antigen. A labeled antibody (e.g. enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and B-galactosidase. The enzyme linked antibody reacts with a substrate to generate a colored reaction product that can be measured.

In a “competitive ELISA”, antibody is incubated with a sample containing antigen (i.e. enzyme). The antigen-antibody mixture is then contacted with a solid phase (e.g. a microtiter plate) that is coated with antigen (i.e., enzyme). The more antigen present in the sample, the less free antibody that will be available to bind to the solid phase. A labeled (e.g., enzyme linked) secondary antibody is then added to the solid phase to determine the amount of primary antibody bound to the solid phase.

In an “immunohistochemistry assay” a test sample is tested for specific proteins by exposing the test sample to antibodies that are specific for the protein that is being assayed. The antibodies are then visualized by any of a number of methods to determine the presence and amount of the protein present. Examples of methods used to visualize antibodies are, for example, through enzymes linked to the antibodies (e.g., luciferase, alkaline phosphatase, horseradish peroxidase, or beta-galactosidase), or chemical methods (e.g., DAB/Substrate chromagen). The sample is then analyzed microscopically, for example, by light microscopy of a sample stained with a stain that is detected in the visible spectrum, using any of a variety of such staining methods and reagents known to those skilled in the art.

Alternatively, “Radioimmunoassays” can be employed. A radioimmunoassay is a technique for detecting and measuring the concentration of an antigen using a labeled (e.g., radioactively or fluorescently labeled) form of the antigen. Examples of radioactive labels for antigens include ³H, and ¹²⁵I. The concentration of antigen enzyme in a test sample or a biological sample can be measured by having the antigen in the biological sample compete with the labeled (e.g. radioactively) antigen for binding to an antibody to the antigen. To ensure competitive binding between the labeled antigen and the unlabeled antigen, the labeled antigen is present in a concentration sufficient to saturate the binding sites of the antibody. The higher the concentration of antigen in the sample, the lower the concentration of labeled antigen that will bind to the antibody.

In a radioimmunoassay, to determine the concentration of labeled antigen bound to antibody, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by performing a “solid-phase radioimmunoassay” where the antibody is linked (e.g., covalently) to Sepharose beads, polystyrene wells, polyvinylchloride wells, or microtiter wells. By comparing the concentration of labeled antigen bound to antibody to a standard curve based on samples having a known concentration of antigen, the concentration of antigen in the biological sample can be determined.

An “Immunoradiometric assay” (IRMA) is an immunoassay in which the antibody reagent is radioactively labeled. An IRMA requires the production of a multivalent antigen conjugate, by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

In some embodiments, Western blotting (Towbin et at., Proc. Nat. Acad. Sci. 76:4350 (1979)) can be used to measure expression levels of specific markers for differentiated muscle or bone cells, wherein a suitably treated sample is run on an SDS-PAGE gel before being transferred to a solid support, such as a nitrocellulose filter. Detectably labeled anti-enzyme antibodies can then be used to assess enzyme levels, where the intensity of the signal from the detectable label corresponds to the amount of enzyme present. Levels can be quantified, for example by densitometry.

In addition to immunoassays, the expression level of at least one of the serum/plasma biomarkers can be determined by mass spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos. 20030199001, 20030134304, 20030077616, which are herein incorporated by reference. Mass spectrometry methods are well known in the art and have been used to quantify and/or identify molecules (see, e.g., Li et al. (2000) Tibtech 18:151-160; Rowley et al. (2000) Methods 20: 383-397; and Kuster and Mann (1998) Curr. Opin. Structural Biol. 8: 393-400).

In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. See, e.g., U.S. Pat. No. 5,118,937 (Hillenkamp et al.), and U.S. Pat. No. 5,045,694 (Beavis & Chait).

In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094. Software programs such as the Biomarker Wizard program (Ciphergen Biosystems, Inc., Fremont, Calif.) can be used to aid in analyzing mass spectra, e.g., comparing the signal strength of peak values from spectra of a test subject sample and a control sample (e.g., a normal healthy person). The mass spectrometers and their techniques are well known to those of skill in the art.

Measuring mRNA Expression:

In some embodiments, the mRNA expression of specific markers for differentiated muscle or bone cells can be measured to quantify muscle growth or bone growth, respectively, of the musculoskeletal cells or precursor cells thereof in response to a test composition. Real time PCR is an amplification technique that can be used to determine expression levels of mRNA corresponding to a protein of interest. (See, e.g., Gibson et al., Genome Research 6:995-1001, 1996; Heid et al., Genome Research 6:986-994, 1996). Real-time PCR evaluates the level of PCR product accumulation during amplification. This technique permits quantitative evaluation of mRNA levels in multiple samples. For mRNA levels, mRNA can be extracted from a biological sample, e.g. cultured cells after contact with a test composition, and cDNA is prepared using standard techniques. Real-time PCR can be performed, for example, using a Perkin Elmer/Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, beta-actin) primers and probes can be obtained commercially from, for example, Perkin Elmer/Applied Biosystems (Foster City, Calif.). To quantitate the amount of the specific nucleic acid of interest in a sample, a standard curve is generated using a control. Standard curves can be generated using the Ct values determined in the real-time PCR, which are related to the initial concentration of the nucleic acid of interest used in the assay. Standard dilutions ranging from 10¹-10⁶ copies of the gene of interest are generally sufficient. In addition, a standard curve is generated for the control sequence. This permits standardization of initial content of the nucleic acid of interest in a test sample to the amount of control for comparison purposes.

Methods of real-time quantitative PCR using TaqMan probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994.

The TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq, results in the cleavage of the TaqMan probe. This cleavage separates the 5′ fluorescent dye and the 3′ quenching agent, thereby resulting in an increase in fluorescence as a function of amplification (see, for example, Perkin-Elmer).

In another embodiment, detection of RNA transcripts can be achieved by Northern blotting, wherein a preparation of RNA is run on a denaturing agarose gel, and transferred to a suitable support, such as activated cellulose, nitrocellulose or glass or nylon membranes. Labeled (e.g., radiolabeled) cDNA or RNA is then hybridized to the preparation, washed and analyzed by methods such as autoradiography.

Detection of RNA transcripts can further be accomplished using known amplification methods. For example, mRNA can be reverse-transcribed into cDNA followed by polymerase chain reaction (RT-PCR); or, to use a single enzyme for both steps as described in U.S. Pat. No. 5,322,770, or reverse transcribe mRNA into cDNA followed by symmetric gap lipase chain reaction (RT-AGLCR) as described by R. L. Marshall, et al., PCR Methods and Applications 4: 80-84 (1994). One suitable method for detecting enzyme mRNA transcripts is described in reference Pabic et al. Hepatology, 37(5): 1056-1066, 2003, which is herein incorporated by reference.

In situ hybridization visualization can also be employed, wherein a radioactively labeled antisense RNA probe is hybridized with target biomarkers in a test sample, washed, cleaved with RNase and exposed to a sensitive emulsion for autoradiography. The samples can be stained with haematoxylin to demonstrate the histological composition of the sample, and dark field imaging with a suitable light filter shows the developed emulsion. Non-radioactive labels such as digoxigenin can also be used.

Alternatively, mRNA expression can be detected on a DNA array, chip or a microarray. Oligonucleotides corresponding to enzyme are immobilized on a chip which is then hybridized with labeled nucleic acids of a test sample obtained from a patient. Positive hybridization signal is obtained with the sample containing biomarker transcripts. Methods of preparing DNA arrays and their use are well known in the art. (See, for example U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

Methods for Using the Assays, Systems, and/Kits Described Herein

Physiologic response to anabolic use is complex and varies with a number of phenotypic and/or genetic factors. See, e.g., Montano et al., Ageing Res Rev 2011, 2011, 10(2): 216-224. For example, physiologic response to anabolic use can be age-dependent. Anabolic use is recognized to increase muscle mass in both young and older individuals. With the progressive aging of the human population, there is a decline in muscle mass, strength and function, a phenomenon that has motivated the use of anabolics, even among those individuals with subclinical decline. While there are biomarkers in serum that can be associated with aging and anabolic use (e.g., testosterone use), and despite similar gains in muscle strength and mass, older men differed from younger men in the serum response profile of these selected biomarkers. In accordance with embodiments of various aspects described herein, the assays, methods, systems and/or kits described herein are developed for generating personalized or stratified anabolic profiles that can account for the existence of complex mechanisms for anabolic response (including, e.g., anabolic resistance), age-sensitivity, as well as other environmental/personalized factors that can influence biomarker robustness. For example, based on a personalized or stratified anabolic profiling (which indicates relative rankings of anabolic efficacies of the test compositions comprising one or more anabolic agents), a test composition can be specifically selected for treatment and/or prevention of a musculoskeletal disease or disorder in a subject or a population subgroup.

Personalized medicine is part of a continuum of care, from one-size-fits-all, to population stratification into subgroups that share features, to individuals as personalized individuals with unique genetic polymorphisms, phenotypic characteristics and/or environmental life histories. Personalized medicine is a treatment model in which patients can benefit from treatment protocols that are tailored to their unique profiles.

In some embodiments, a personalized muscle anabolic profiling can be generated by stratifying a cohort into subpopulations, for example, based on at least one or more stratification features, including, e.g., but not limited to, genetic polymorphisms, phenotypic characteristics and/or environmental life histories, to identify differential anabolic response among different subpopulations. Examples of stratification features include, but are not limited to, age groups, gender, ethnicity (e.g., Caucasians vs. Asians or African Americans), body types, body mass index (BMI), blood types, activity levels (e.g., sedentary work or lifestyle vs. athletic or active lifestyle), chronic and/or acute diseases or disorders, diet or nutrition, habits (e.g., smoking, alcoholic), drug resistance, treatment regime such as chemotherapy, drastic/abnormal weight loss, genetic polymorphisms, geographical locations, and any combinations thereof. The anabolic profiles of these strata can be generated using one or more embodiments of the assay described herein, where the musculoskeletal or precursor cells thereof used in the assay include a panel of cells representing different population subgroups. By way of example only, in one embodiment, muscle precursor stem cells (MPCs) from distinct individuals (e.g., young vs. old, male vs. female), can be screened against a selected panel of test compositions or anabolic agents chosen to achieve broad coverage of known anabolic pathways as described herein. The muscle and/or bone growth response of the MPCs for each test composition or anabolic agent can be quantitatively measured using one or more embodiments as described above, thereby generating an anabolic growth diagnostic report for subpopulations, for example, based on age and sex. An individual can then be mapped to a specific population subgroup based on certain characteristics of the individual, and obtain an anabolic profile representing the population subgroup to which the individual is belonged, without performing a personal biopsy. By way of example only, an individual can be mapped to a specific population subgroup based on age, gender, and/or ethnicity. By integrating patient specific information into a treatment selection process for subjects who are in need of muscle augmentation and/or mitigation of muscle and/or bone loss (including ones who are in need for treatment of a musculoskeletal disease or disorder), a stratified diagnostics and therapeutics can be provided to complement standard-of-care based guidelines.

In some embodiments, a more personalized anabolic profiling can be generated based on a muscle biopsy- or blood sample-derived muscle and/or bone precursor cells thereof specific for an individual.

In some embodiments, an individual can be mapped to a population subgroup to first identify a subset of anabolic agents that the individual are likely to respond. A more personalized anabolic profiling specific for the subset of anabolic agents can be generated based on the individual's muscle biopsy- or blood sample-derived muscle and/or bone precursor cells thereof.

In some embodiments, stratification profiles can be prepared as discussed above. These stratification profiles can be used in the sale of anabolic products. Thus, for example, one particular product may work better for women over 50 years old than another product, which might be better for women under 50 years old. This information could be included on the product in a health food store where products can be displayed by such characteristics. The information could also be provided by a smart phone application.

Accordingly, another aspect provided herein relates to a method of optimizing or selecting a treatment regimen for a subject determined to have, or have a risk for, a musculoskeletal disease or disorder, where the method comprises performing one or more embodiments of the assay described herein. For example, the method can comprise subjecting the musculoskeletal cells or precursor cells thereof to one or more embodiments of the assays, systems and/or kits described herein, wherein the musculoskeletal cells or precursor cells thereof can be obtained or derived from (i) a subject determined to have, or have a risk for, a musculoskeletal disease or disorder; or from (ii) a group of individuals sharing a similar background and symptoms as the subject (based on at least one or at least two or more features such as phenotypic features described herein). The test compositions can be ranked based on its efficacy to stimulate muscle and/or bone growth as determined in the assay. In one embodiment, the test composition can be ranked based on its efficacy to stimulate muscle growth, for example, characterized by a fusion index as described herein. If some of the test compositions show an anabolic efficacy above a pre-determined anabolic threshold or a level of a control or reference (e.g., anabolic response of the musculoskeletal or precursor cells thereof in the absence of the test composition), at least one of those test compositions can be selected, based on their ranking in the assay described herein, for administration to the subject. If none of the test compositions demonstrates an anabolic efficacy above the pre-determined threshold, none of the test compositions is selected or recommended for the treatment.

In some embodiments of various aspects described herein where anabolic efficacy of a test composition is determined based on the ability of the test composition to induce fusion of the musculoskeletal cells or precursor cells thereof to form multi-nucleated cells, the anabolic threshold can be defined as the frequency of three or more nuclei per at least a subset of the cells used in the assay. In one embodiment, the anabolic threshold can be defined as the frequency of three or more nuclei per ˜100 cells, based on analysis of ˜1000 cells in replicate. Cells with one nucleus per cell or two nuclei per cell would be excluded and considered as sub-threshold random variation.

By employing one or more embodiments of the assays, systems and/or kits described herein, selection or optimization of a treatment regimen can include, but is not limited to, selection of a specific anabolic agent or combination therapy that stimulate muscle and/or bone growth in the subject, optimization of the dosage and/or administration schedule of the selected anabolic agent(s) for a personalized treatment, or both.

Methods of treating a subject determined to have, or have a risk for, a musculoskeletal disease or disorder are also provided herein. In one embodiment, the method comprises performing one or more embodiments of the assay described herein. For example, the method can comprise subjecting the musculoskeletal cells or precursor cells thereof to one or more embodiments of the assays, systems and/or kits described herein, wherein the musculoskeletal cells or precursor cells thereof can be obtained or derived from (i) a subject determined to have, or have a risk for, a musculoskeletal disease or disorder; or from (ii) a group of individuals sharing a similar background and symptoms as the subject (based on at least one or at least two or more features such as phenotypic features described herein). The test compositions can be ranked based on its efficacy to stimulate muscle and/or bone growth as determined in the assay. In one embodiment, the test composition can be ranked based on its efficacy to stimulate muscle growth, for example, characterized by a fusion index as described herein. If some of the test compositions show an anabolic efficacy above a pre-determined threshold or a level of a control or reference (e.g., anabolic response of the musculoskeletal or precursor cells thereof in the absence of the test composition), at least one of those test compositions can be selected, based on their ranking in the assay described herein, to treat the subject. In such embodiments, the method can further comprise prescribing or administering an effective amount of the selected test composition to the subject. However, if none of the test compositions demonstrates an anabolic efficacy above the pre-determined threshold, none of the test compositions is selected or recommended for the treatment.

In another embodiment, the method of treating a subject determined to have, or have a risk for, a musculoskeletal disease or disorder can comprise prescribing or administering an effective amount of a test composition to the subject who is determined to have or have a risk for a musculoskeletal disease or disorder, wherein the test composition was selected based upon its ranking in one or more embodiments of the assay described herein. The term “effective amount” as used herein refers to an amount sufficient to increase the muscle growth and/or bone growth as described herein, by at least about 10% or higher, as compared to the muscle growth and/or bone growth without administration of the test composition.

The terms “treatment” and “treating” as used herein, with respect to treatment of a disease, means preventing the progression of the disease, or altering the course of the disorder (for example, but are not limited to, slowing the progression of the disorder), or reversing a symptom of the disorder or reducing one or more symptoms and/or one or more biochemical markers in a subject, preventing one or more symptoms from worsening or progressing, promoting recovery or improving prognosis. For example, in the case of treating a musculoskeletal disease or disorder, therapeutic treatment refers to alleviation of at least one symptom associated with the musculoskeletal disease or disorder. Measurable lessening includes any statistically significant decline in a measurable marker or symptom, such as a reduction in muscle wasting and/or bone loss, and/or an increase in muscle and/or bone strength, after treatment. In one embodiment, at least one symptom of a musculoskeletal disease or disorder is alleviated by at least about 10%, at least about 15%, at least about 20%, at least about 30%, at least about 40%, or at least about 50%. In another embodiment, at least one symptom is alleviated by more than 50%, e.g., at least about 60%, or at least about 70%. In one embodiment, at least one symptom is alleviated by at least about 80%, at least about 90% or greater, as compared to a control (e.g., a subject having the same condition as the treated subject is administered without the test composition, or a subject whose anabolic profile does not recommend the test composition is administered with the test composition). In one embodiment, at least one marker associated with a musculoskeletal disease or disorder (e.g., creatine kinase) is alleviated by at least about 10%, at least about 15%, at least about 20%, at least about 30%, at least about 40%, or at least about 50%. In another embodiment, at least one marker associated with a musculoskeletal disease or disorder (e.g., creatine kinase) is alleviated by more than 50%, e.g., at least about 60%, or at least about 70%. In one embodiment, at least one marker associated with a musculoskeletal disease or disorder (e.g., creatine kinase) is alleviated by at least about 80%, at least about 90% or greater, as compared to a control (e.g., a subject having the same condition as the treated subject is administered without the test composition, or a subject whose anabolic profile does not recommend the test composition is administered with the test composition).

While some embodiments of the assays described herein can be employed as part of treatment of a musculoskeletal disease or disorder, other embodiments of the assays described herein can be employed as part of preventive care in individuals seeking to mitigate or prevent loss in muscle and bone, e.g., on a routine basis to extend health-span. Accordingly, methods of preventing a musculoskeletal disease or disorder in a subject are also provided herein. As used herein, the term “preventing” with respect to a condition or disorder refers to delaying or preventing the onset of a musculoskeletal disease or disorder described herein, or the onset of a muscle and/or bone loss, e.g., in a subject at risk of having a musculoskeletal disease or disorder, and/or a muscle and/or bone loss. In some embodiments, “preventing” a condition can also encompass inhibiting, decreasing, or slowing the progression or severity of the condition, e.g., in a subject being diagnosed with the condition. The onset, the progression or severity of such disorder or condition can be determined by detecting an increase in at least one symptom associated with the condition, or a decrease in the muscle/bone mass and/or function affected by the condition. Such detection methods for any muscle and/or bone loss as well as musculoskeletal disease or condition are known in the art, e.g., by imaging (e.g., X-ray, MRI, CT scan), electromyography, and blood/urine test for measuring expression levels of disorder-specific biomarkers (e.g., creatine kinase, urea).

In one embodiment, the method of preventing a musculoskeletal disease or disorder in a subject, or maintaining or increasing muscle and/or bone mass in a subject can comprise performing one or more embodiments of the assay described herein. For example, the method can comprise subjecting the musculoskeletal cells or precursor cells thereof to one or more embodiments of the assays, systems and/or kits described herein, wherein the musculoskeletal cells or precursor cells thereof can be obtained or derived from (i) a subject determined to have, or have a risk for, a muscle and/or bone loss, or experience at least one symptom associated with an onset of a muscle and/or bone loss; or from (ii) a group of individuals sharing a similar background and symptoms as the subject (based on at least one or at least two or more features such as phenotypic features described herein). The test compositions can be ranked based on its efficacy to stimulate muscle and/or bone growth as determined in the assay. In one embodiment, the test composition can be ranked based on its efficacy to stimulate muscle growth, for example, characterized by a fusion index as described herein. If some of the test compositions show an anabolic efficacy above a pre-determined threshold or a level of a control or reference (e.g., anabolic response of the musculoskeletal or precursor cells thereof in the absence of the test composition), it indicates that a subset of the test composition can reduce or delay muscle and/or bone loss. In these embodiments, at least one of those test compositions can be selected and recommended, based on their ranking in the assay described herein, as a preventative care or supplement. In such embodiments, the method can further comprise prescribing or administering an effective amount of the selected test composition to the subject. However, if none of the test compositions indicates a reduction or delay in muscle and bone loss, none of the test compositions is selected or recommended.

In another embodiment, the method of preventing a musculoskeletal disease or disorder in a subject, or maintaining or increasing muscle and/or bone mass can comprise prescribing or administering an effective amount of a test composition to the subject who is determined to have a loss in muscle and/or bone, or experience at least one symptom associated with an onset of a loss in muscle and/or bone, wherein the test composition was selected based upon its ranking in one or more embodiments of the assay described herein. In one embodiment, the effective amount administered to a subject is an amount sufficient to slow down a decrease in muscle and/or bone mass by at least about 10%, at least about 20%, at least about 30%, at least about 40%, at least about 50%, at least about 60%, at least about 70%, at least about 80%, at least about 90%, or higher, as compared to the muscle and/or bone loss without administration of the selected test composition. In one embodiment, the effective amount administered to a subject is an amount sufficient to maintain the muscle and/or bone condition (e.g., no significant change in the muscle and/or bone condition) over a period of time, e.g., at least about 1 month, at least about 2 months, at least about 3 months, at least about 6 months, or longer.

In some embodiments of the methods of various aspects described herein, the composition with the highest rank (i.e., highest anabolic efficacy, if the test compositions are ranked in a descending order of anabolic efficacy) with respect to the muscle and/or bone growth as determined in the assay described herein can be selected and administered to the subject. Stated another way, in some embodiments, the composition that works best for a particular population of individuals with respect to muscle and/or bone growth as determined from a stratification profile based upon using the assay described herein can be selected and administered to the subject. In other embodiments, other factors such as side effects and/or price of the drug, and/or other drugs that the subject is taking can be considered when selecting the test composition for treating the subject. In such embodiments, the test composition with a lower rank and an anabolic efficacy above a pre-determined threshold (e.g., anabolic response of the musculoskeletal or precursor cells thereof in the absence of the test composition) can be selected and administered to the subject instead.

In some embodiments of the methods of any aspects described herein, a skilled practitioner (e.g., a clinical advisor) can provide insight into the analysis of pro-anabolic compounds influencing muscle growth and any potential contra-indications of selected compounds, based on their rank in the resultant anabolic profile. Not only can the anabolic profiles generated by the assays, systems and/or kits described herein provide personalized or stratified information about which test composition indicates a higher anabolic efficacy for a specific subject or a subset of population, but it can also determine anabolic resistance of the specific subject or the subset of population. For example, if a subset of the test compositions associated with a specific anabolic pathway score a low rank (corresponding to low anabolic efficacy if the test compositions are ranked in a descending order of anabolic efficacy) and/or do not reach a pre-determined threshold value of anabolic efficacy, it indicates that the specific subject or the subset of population can develop an anabolic resistance to the molecules associated with the specific anabolic pathway, and/or can experience an imbalance among the anabolic pathways. Accordingly, methods for determining an anabolic resistance in a subject or a subset of populations are also provided herein. The method comprises subjecting the musculoskeletal cells or precursor cells thereof obtained or derived from a subject or a subset of populations to one or more embodiments of the assays, systems, and/or kits described herein. When the anabolic efficacy of at least one of the test compositions is determined to be below a pre-determined threshold, it indicates that the subject is or the subset of the population are non-responsive or resistant to the at least one of the test compositions.

In some embodiments, the methods of various aspects described herein do not necessarily require a biological sample from a subject to perform the assay as described herein. Instead, a database comprising anabolic profiles for a plurality of population subgroups stratified by at least one feature such as phenotypic feature as described herein can be created and established. Thus, a subject seeking an anabolic treatment or supplement can be matched or associated to one of the population subgroups in the database based on at least one feature such as phenotypic feature (e.g., age, gender, ethnicity, condition, and/or BMI), thereby selecting an anabolic agent based on the rankings of the anabolic agents for the matching population subgroup. Accordingly, in another aspect, provided herein is a method of selecting an anabolic agent for a subject in need of anabolic augmentation and/or mitigation of muscle and/or bone loss. The method comprises (a) providing or creating a database comprising anabolic information for a plurality of population subgroups stratified or characterized by at least one feature, wherein the anabolic information for each of the population subgroups comprises rankings of a plurality of anabolic agents based on their anabolic efficacy in each of the population subgroups; and (b) mapping or associating a subject who is in need of anabolic augmentation or muscle loss reduction to one of the plurality of the population subgroups based on the at least one feature such as phenotypic feature, thereby selecting at least one anabolic agent for the subject based on the ranking of the anabolic agents in the matching population subgroup.

In some embodiments, the method can further comprise providing a computer system or processing device, the computer system or processing device including a processor and associated memory, a user input component and an output component. In these embodiments, the method can further comprise connecting the computer system or processing device to the database. Examples of the computer or processor device include, but are not limited to, a personal digital assistant (PDA), smart-phone, cellular phone, a computer, a tablet PC, and any combination thereof.

In some embodiments, the method can further comprise, prior to the mapping or association, inputting into the computer system or processing device at least one feature associated with the subject in need of an anabolic treatment (e.g., a subject who is in need of anabolic augmentation or mitigation of muscle loss or bone loss).

In some embodiments, the mapping or association of the subject to a specific population subgroup can further comprise searching the database for anabolic information of an associated population subgroup characterized by the input feature. Examples of a subject's feature can include phenotypic features for population stratification including, but not limited to, age or age groups, gender, ethnicity or races, body types, condition, weights, heights, body mass index (BMI), blood types, activity levels (e.g., sedentary lifestyle or work such as a secretary in office vs. active lifestyle or work such as an athlete), chronic or acute diseases (e.g., but not limited to, diabetes, cancer, osteoporosis, HIV infection, infection, musculoskeletal diseases or disorders, metabolic diseases or disorders, and psychophysiological disorders), genetic polymorphisms, diet (e.g., but not limited to, vegetarian, and gluten-free), living habits (e.g., but not limited to, smoking and alcohols), drug resistance, treatment regime such as chemotherapy, drastic/abnormal weight loss, geographical location (e.g., individuals living in the west coast vs. east coast of the United States, or individuals living in the United States vs. in Asian countries) and environmental factors associated therewith, and any combinations thereof.

In some embodiments, the method can further comprise selecting the at least one anabolic agent for the subject based on the ranking of the anabolic agents in the associated population subgroup. In some embodiments, the method can further administering to the subject the selected anabolic agent. Accordingly, provided herein is also a method of treating a subject who is in need of anabolic augmentation and/or mitigation of muscle and/or bone loss, which comprises administering at least one selected anabolic agent to the subject, wherein the at least one selected anabolic agent is determined based on a process comprising: (a) providing a database comprising anabolic information for a plurality of population subgroups stratified or characterized by at least one feature such as a phenotypic feature, wherein the anabolic information for each of the population subgroups comprises rankings of a plurality of anabolic agents based on their anabolic efficacy in each of the population subgroups; and (b) mapping the subject to one of the plurality of the population subgroups based on the at least one feature such as the phenotypic feature, thereby selecting the at least one anabolic agent for the subject based on the ranking of the anabolic agents in the matching population subgroup.

In some embodiments, the stratification profiles can be used in the sale of anabolic agents or products. Thus, for example, one particular anabolic agent or product may work better for women over 50 years old than another product, which might be better for women under 50 years old. As another example, a specific anabolic agent or product (e.g., a product that does not require an insulin pathway) may work better for a diabetic subject than another product which requires an insulin pathway. This kind of information can be included on the packaging of the anabolic agent or product in a health food store where products can be displayed by such characteristics. This kind of information can also be included on the packaging of prescription or over-the-counter anabolic drugs in a pharmacy store. The information could also be provided by a smart phone application.

In some embodiments, the anabolic efficacy of the anabolic agents can be determined based on the effect of the anabolic agents on fusion of muscle precursor cells to form multi-nucleated cells. Additionally or alternatively, the anabolic efficacy of the anabolic agents can be determined based on the effect of the anabolic agents on differentiation of muscle cells or bone precursor cells to bone cells. Accordingly, in some embodiments, the database can be created by a method comprising: (a) for each of the plurality of the population subgroups, quantifying muscle growth and/or bone growth of the musculoskeletal cells or precursor cells thereof obtained or derived from the population subgroup, upon the contact of the musculoskeletal cells or precursor cells thereof with the plurality of the anabolic agents; and (b) ranking anabolic efficacy of the plurality of the anabolic agents based on the quantified muscle growth and/or bone growth for each of the plurality of the population subgroups.

The database used in the method described herein can comprise anabolic profiles of at least 2 or more population subgroups, including, e.g., 2, 3, 4, 5, 6, 7, 8, 9, 10, 20, 30, 40, 50, 60, 70, 80, 90, 100, 150, 200, 250, 300, 350, 400, 450, 500 or more distinct population subgroups, depending on the size of the database (e.g., the number and/or diversity of the individuals). For example, the population of individuals can be stratified into two subgroups by gender (i.e., male vs. female), and each subgroup can be further stratified into smaller groups based on age or age groups, ethnicity, condition and/or body mass index (BMI). In some embodiments, the population of individuals can be stratified based on disease symptoms experienced by the individuals. In some embodiments, the population of individuals can be stratified based on a treatment regime such as chemotherapy taken by the subject.

In some embodiments, the subjects amenable to the methods of any aspects described herein can include, but are not limited to, individuals suffering or having a risk for a musculoskeletal disease or disorder, athletes, aging individuals, individuals having a chronic disease or disorder (e.g., but not limited to, cancer, chronic obstructive pulmonary disease (COPD), chronic kidney disease (CKD), chronic liver failure (CLF), and chronic infections), individuals suffering from malnutrition, individuals afflicted with HIV infection, cancer survivors, individuals showing excessive weight loss, individuals that have previously shown non-responsiveness or resistance to at least one or more anabolic agents, or any combinations thereof.

The agent(s) included in the test compositions can include a therapeutic agent that has already been indicated for anabolic treatment, and/or a candidate agent to be assessed for its anabolic efficacy. The test compositions used in the assay described herein can each independently comprise one or more agents selected to increase and/or maintain muscle and/or bone growth. In some embodiments, at least some of the test compositions can comprise two or more agents selected to increase and/or maintain muscle and/or bone growth. The agent(s) included in the test compositions can include a therapeutic agent that has already been indicated for anabolic treatment (e.g., FDA-approved anabolic drugs or over-the-counter anabolic drugs), off-label FDA-approved drugs or over-the-counter drugs, an anabolic supplement, a candidate agent to be assessed for its anabolic efficacy, or any combinations thereof. As such, while in some embodiments, the assays described herein can be used to select or optimize a treatment regimen for a subject with a musculoskeletal condition, in some embodiments, the assays described herein can be used to identify a novel anabolic compound specific for a subject's or a population subgroup's musculoskeletal condition, e.g., as shown in Example 3.

Selection of Subjects with a Musculoskeletal Disease or Disorder

In accordance with some embodiments described herein, subjects amenable to assays, methods, compositions, and kits described herein are subjects who are in need of muscle augmentation and/or mitigation of muscle and/or bone loss. As used herein, by the term “muscle augmentation” is meant increasing the muscle mass and/or strength. In some embodiments, the muscle mass and/or strength can be increased by at least about 10%, at least about 20%, at least about 30%, at least about 40%, at least about 50%, at least about 60%, at least about 70%, at least about 80%, at least about 90% or higher, when a subject is administered with an anabolic agent selected based on its anabolic efficacy as determined in the assay described herein, as compared to treatment without the selected anabolic agent. In some embodiments, the term “muscle augmentation” can also refer to decreasing, reducing or alleviating at least one symptom associated with a loss in muscle mass and/or strength. For example, an increased muscle mass and/or strength can reduce a decline in functional mobility due to a loss in the muscle mass. Thus, in some embodiments, muscle augmentation can be characterized by an increase in functional mobility.

In some embodiments, the muscle loss experienced by the subject can be due to aging. Accordingly, in some embodiments, the subject who is in need of muscle augmentation and/or mitigation of muscle and/or bone loss can be an aging subject. Loss of muscle mass is increasingly common in aging. There are currently 70 million individuals born in the United States (US) from 1946 to 1964 (age 49-to-67) referred to as the “baby boomers” and 40.3 million people who are 65+ years old. As the US population ages, there is an increasing prevalence of muscle loss, raising the risk for frailty, declines in functional mobility, and early mortality. The National Health and Nutrition Examination Survey (NHANES) estimates a prevalence of sarcopenia (decline in muscle mass and function) of approximately 7-10%.

Age-associated declines in muscle mass and muscle strength is increasingly common. Skeletal muscle accounts for 40-50% of total body mass, and is critical for both mobility and bioenergetic metabolism. Muscle homeostasis represents a dynamic balance between anabolism and catabolism of muscle, mediated by the actions of growth factors and cytokines, as well as their respective regulators, with a potential role for vasculature in providing growth maintenance cues (e.g., insulin). The loss of muscle mass and/or strength is a universal feature of aging. In many cases, a loss in muscle mass can raise the risk for frailty and thus impair functional capacity leading to disability and even early mortality. Accordingly, in some embodiments, the subject who is in need of muscle augmentation and/or mitigation of muscle and/or bone loss can be an aging subject who is seeking to mitigate age-associated functional decline.

The term “sarcopenia” as used herein is used to describe the wasting effects of age on skeletal muscle, typically characterized by a loss of muscle mass and/or function, metabolic dysregulation, and/or an overall increase in vulnerability to stressors, e.g., in the context of co-morbidities.

In some embodiments, the subject who is in need of muscle augmentation and/or mitigation of muscle and/or bone loss can be a subject suffering from malnutrition. Malnutrition can contribute to muscle loss. For example, there can be an imbalance between energy intake and energy expenditure, with a growing deficit in protein intake. Accordingly, in some embodiments, the assays, methods, systems, and/or kit described herein can be used to identify and/or optimize a nutritional option for treatment of a muscle loss and/or bone loss. Without wishing to be bound by theory, this anabolic resistance can be due to a “desynchronization” of pathways for protein utilization and thus result in loss in muscle mass. The anabolic profiles as generated in the assay described herein can facilitate determining one or more specific pathways that appear to be desynchronized from others or that are defective or non-responsive and thus identify a more effective approach to counter muscle loss.

In some embodiments, the subject who is in need of muscle augmentation and/or mitigation of muscle and/or bone loss can be an athlete who needs to strengthen and/or build more muscles.

In some embodiments, the subject who is in need of muscle augmentation and/or mitigation of muscle and/or bone loss can be diagnosed with or suspected of having or developing a musculoskeletal disease or disorder. The phrase “suspected of having or developing a musculoskeletal disease or disorder” refers to a subject that presents one or more symptoms indicative of a risk for a musculoskeletal disease or disorder (e.g., muscle wasting, bone loss, fatigue, pain, tenderness, impairment in mobility, soft tissue swelling, or bony swelling etc.). Accordingly, in some embodiments, subjects that have been diagnosed or suspected of having or developing with a musculoskeletal disease or disorder are selected prior to subjecting them to the assays, methods, compositions and kits described herein. In some embodiments, a subject selected for the assays, methods, compositions and kits described herein is being treated for the diagnosed musculoskeletal disease or disorder. For example, where a subject with a musculoskeletal disease or disorder is being administered with a therapeutic agent for treatment of the musculoskeletal disease or disorder, the subject can be selected for the assays, methods, and kits described herein, e.g., for optimizing the current treatment regimen (e.g., dosage and administration frequency) and/or selecting an alternative treatment regimen (e.g., a different anabolic agent) to increase the therapeutic outcome. Accordingly, a subject amenable to the assays, methods and/or compositions described herein is specifically selected for any musculoskeletal disease or disorder before performing the assays and/or methods described herein and/or administering the compositions described herein.

By “musculoskeletal disease or disorder” is meant a disease or disorder of the muscles, ligaments, bones, joints, cartilage, and/or other connective tissue. Among the most commonly-occurring musculoskeletal disorders are various forms of arthritis, e.g., osteoarthritis, rheumatoid arthritis, juvenile rheumatoid arthritis, and gout. Other musculoskeletal disorders include acquired hyperostosis syndrome, acromegaly, ankylosing spondylitis, Behcet's disease, bone diseases, bursitis, cartilage diseases, chronic fatigue syndrome, compartment syndromes, congenital hypothyroidism, congenital myopathies, dentigerous cyst, dermatomyositis, diffuse idiopathic skeletal hyperostosis, Dupuytren's contracture, eosinophilia-myalgia syndrome, fasciitis, Felty's syndrome, fibromyalgia, hallux valgus, infectious arthritis, joint diseases, Kabuki make-up syndrome, Legg-Perthes disease, lupus, Lyme disease, Melas syndrome, metabolic bone diseases, mitochondrial myopathies, mixed connective tissue disease, muscular diseases, muscular dystrophies, musculoskeletal abnormalities, myositis, myositis ossificans, necrotizing fasciitis, neurogenic arthropathy, osteitis deformans, osteochondritis, osteomalacia, osteomyelitis, osteonecrosis, osteoporosis, Paget's disease, Pierre Robin syndrome, polymyalgia rheumatica, polymyositis, postpoliomyelitis syndrome, pseudogout, psoriatic arthritis, reactive arthritis, Reiter disease, relapsing polychondritis, renal osteodystrophy, rhabdomyolysis, rheumatic diseases, rheumatic fever, scleroderma, Sever's disease (calceneal apophysitis), Sjögren's syndrome, spinal diseases, spinal stenosis, Still's disease, synovitis, temporomandibular joint disorders, tendinopathy, tennis elbow, tenosynovitis, Tietze's syndrome, and Wegener's granulomatosis, muscle wasting associated with HIV-infection, cachexia, muscular dystrophy, osteopenia, sarcopenia, an age-related musculoskeletal disease or disorder, or a musculoskeletal disease or disorder associated with anabolic assistance.

In some embodiments, a musculoskeletal disease or disorder amenable to the methods of treatment described herein is associated with an immune response. In some embodiments, a musculoskeletal disease or disorder amenable to the methods of treatment described herein includes muscle wasting associated with HIV infection. In some embodiments, a musculoskeletal disease or disorder amenable to the methods of treatment described herein includes cachexia, muscular dystrophy, osteopenia, osteoporosis, sarcopenia, an age-related musculoskeletal disease or disorder, or a musculoskeletal disease or disorder associated with anabolic resistance.

In some embodiments, a musculoskeletal disease or disorder amenable to the methods of treatment described herein can include a “muscle wasting associated disorder or condition,” which can encompass any disorders or conditions in which muscle wasting or loss of muscle is one of the symptoms or is one of the primary symptoms. For example, loss of lean muscles can be a comorbid condition in multiple chronic, acute and/or psychophysiological diseases or disorders such as muscular dystrophy, spinal cord injury, neurodegenerative diseases, anorexia, sarcopenia, cachexia, cancer cachexia, HIV-associated weight loss, inflammatory sepsis, muscular atrophy due to immobilization, prolonged bed rest, or weightlessness, and the like, as well as disorders in which an abnormally high fat-to-muscle ratio is implicated in a disease or pre-disease state, e.g., Type II diabetes or Syndrome X. Accordingly, in some embodiments, subjects who are in need of muscle augmentation and/or mitigation of muscle and/or bone loss are subjects having a comorbid condition of increased risk for wasting/cachexia as a side effect of a chronic disease state. In some embodiments, the subjects with a managed chronic disease but suffering from or having a risk for wasting/cachexia can be amenable to the methods described herein, thereby determining an optimum treatment option for them to maintain and/or increase their muscle mass.

The term “cachexia” as used herein is derived from the Greek meaning “bad condition”. This condition is generally recognized as a metabolic syndrome associated with underlying illness and substantial loss in muscle mass with or without loss in fat mass. Cachexia can be a severe condition in association with diseases such as cancer, chronic obstructive pulmonary disease (COPD) chronic kidney disease (CKD), chronic liver failure (CLF) and chronic infections. In the case of cancer cachexia, the syndrome can include loss in muscle and optionally fat mass that cannot be restored by nutritional support. Mortality associated with cachexia generally ranges between 20-40%. Protein catabolism in cachexia can offset gains in muscle due to anabolic activity. The initial protein targets that are catabolized in muscle appear to be the myofilaments, a threadlike structure that contains myofibrils composed of striated muscle fibers. Single nucleotide polymorphisms (SNPs) in IL-1, IL-6, IL-10 have been discussed to be associated with prevalence of cachexia. The 1082G allele in the IL-10 promoter has been discussed to be associated with a procachectic genotype and reduced survival, presumably due to altered IL-10 levels. In ˜40% of cancer patients that are obese, there is an observed sarcopenic underlaying phenotype, sarcopenic obesity. Sarcopenic obesity is also observed in other conditions such as Type 2 Diabetes Mellitus (T2DM). This phenomenon can represent a vicious cycle wherein adipose tissue produces inflammatory factors that drive muscle catabolic pathways. Without wishing to be bound by theory, several signaling pathways have been discussed to contribute to loss in muscle mass associated with cachexia, including, but are not limited to, (1) the ubiquitin proteasome system, (2) the calpain protease system, (3) the lysosomal proteolysis pathway, (4) increased myostatin expression, (5) reduced MyoD expression and reduced IGF1 expression, and any combinations thereof.

In some embodiments, subjects who are amenable to the assays, methods, compositions and kits described herein are subjects who have shown anabolic non-responsiveness or resistance to at least one or more (including, e.g., at least two, at least three or more) anabolic agents previously administered to them. Patient response to anabolic agents is variable and at present there is no way to identify patients in advance that are likely to respond favorably (or not) to a particular treatment. In 2011, consumers spent approximately $1.6 billion on prescription testosterone therapies. However, nearly 20% of patients may not respond fully to testosterone supplementation. Similarly, the use of human growth hormone (GH) has also been characterized by considerable variability in muscle growth response. Factors that can contribute to treatment variability include, but are not limited to, when the treatment is initiated, the severity of disease, genetic variation in anabolic signaling intermediates (e.g., functional polymorphisms) and/or genetic variability in physical function capacity in young subjects and older subjects. Embodiments of various aspects described herein provide information on muscle anabolic profiling, which can be in turn used in decision support to better match patients with optimally effective treatment options.

Methods for assessing the condition of muscle and bone in a subject are known in the art. For example, plasma creatine phosphokinase (CPK) is measured by ELISA, with confirmatory cases using muscle biopsy to measure CPK and phosphocreatine (PCr) by magnetic resonance spectroscopy (MRS) to measure muscle wasting. Urine urea is used to infer rapid loss of muscle. Electromyography (EMG) is used to measure neuromuscular function using a surface electrode. Standardized reference values for body composition (e.g., NHANES) are used in assessing loss of skeletal muscle. Body composition assessments through the use of CT image analysis or dual x-ray absorptiometry (DXA) may be used to quantify loss of skeletal muscle or bone, respectively.

Additionally, currently available techniques for the noninvasive assessment of the skeleton for the diagnosis of a musculoskeletal disease or disorder in various tissues (e.g., hip, spine, trabecular bone, peripheral skeleton) or the evaluation of an increased risk of fracture include dual x-ray absorptiometry (DXA), e.g., for measuring bone mineral density (Eastell et al. (1998) New Engl J. Med 338:736-746); quantitative computed tomography (QCT) (Cann (1988) Radiology 166:509-522); peripheral DXA (PDXA) (Patel et al. (1999) J Clin Densitom 2:397-401); peripheral QCT (PQCT) (Gluer et. al. (1997) Semin Nucl Med 27:229-247); x-ray image absorptiometry (RA) (Gluer et. al. (1997) Semin Nucl Med 27:229-247); and quantitative ultrasound (QUS) (Njeh et al. “Quantitative Ultrasound: Assessment of Osteoporosis and Bone Status” 1999, Martin-Dunitz, London England; U.S. Pat. No. 6,077,224, incorporated herein by reference). (See, also, WO 9945845; WO 99/08597; and U.S. Pat. No. 6,246,745).

In some embodiments, subjects amenable to assays, methods, compositions and kits described herein are subjects that have been diagnosed with or suspected of having or developing a musculoskeletal disease or disorder described herein, e.g., HIV infection-associated muscle wasting.

In some embodiments, subjects amenable to assays, methods, compositions and kits described herein are subjects that have been diagnosed with or suspected of having or developing anabolic resistance (e.g., which develops with aging and/or defects or “desynchronization” of pathways for protein utilization and/or muscle growth) that represents a barrier to therapeutic intervention.

In some embodiments, the subject selected for the assays, methods, compositions and kits described herein have been in remission from a musculoskeletal disorder and is now diagnosed with a relapse or a predisposition to a relapse. In other embodiments, the subject selected for the assays, methods and compositions described herein have been diagnosed with a musculoskeletal disease or disorder and is currently taking at least one anabolic agent.

As used herein, a “subject” can mean a human or an animal. Examples of subjects include primates (e.g., humans, and monkeys). Generally, the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. A patient or a subject includes any subset of the foregoing, e.g., all of the above, or includes one or more groups or species such as humans, primates or rodents. In certain embodiments of the aspects described herein, the subject is a mammal, e.g., a primate, e.g., a human. The terms, “patient”, “individual”, and “subject” are used interchangeably herein.

In some embodiments, the human subjects amenable to the assays, methods, compositions and kits described herein can be of any age. In some embodiments, the human subjects amenable to the assays, methods, compositions and kits described can be at an age of at least 18 years old. In other embodiments, human subjects below 18 years can also be subjected to the assays, methods, compositions and kits described herein.

### Treatment Regimen Comprising a Test Composition Selected Based on its Ranking in an Assay Described Herein

A selected test composition in a treatment regimen can be administered together via a single dosage form or by separate administration of each active ingredient or agent encompassed by the selected test composition. In certain embodiments, the selected test composition can be administered together in a single dosage form. For example, the single dosage form can be administered as a single tablet, pill, capsule for oral administration or a solution for parenteral administration. Alternatively, the selected test composition comprising more than one anabolic agents can be administered as separate components, e.g., as separate tablets or solutions. In some embodiments where the selected test composition is administered in separate components, different components can be administrated by the same or different routes. For example, one component can be administered by intravenous or intramuscular injection while another component can be administered orally, or vice versa. Alternatively, for example, all the separate components can be administered together by the same route, e.g., but not limited to, intravenous or intramuscular injection or by oral administration.

In some embodiments, the treatment regimen can further comprise life-style advice, including, e.g., prescribing an exercise regime, dietary advice, and/or administering another pharmaceutical agent effective in treatment of a musculoskeletal disease or disorder.

Examples of the Test Compositions and/or Anabolic Agents

The term “test composition” as used herein refers to a composition comprising at least one or more anabolic agents. As used herein, the term “anabolic agent” refers to an agent selected to increase and/or maintain muscle and/or bone growth in at least one subject or at least one population subgroup. In some embodiments, the anabolic agents included in the test compositions can be agents that have been indicated for anabolic treatment. In some embodiments, the anabolic agents included in the test compositions can be off-label FDA-approved and/or over-the-counter drugs or supplements. In some embodiments, the anabolic agents included in the test compositions can be candidate agents for anabolic treatment. In some embodiments, the anabolic agents included in the test compositions can be anabolic/nutritional supplements, e.g., which can be found in a health food store. The anabolic agent can include, but are not limited to, nutritional supplementation, FDA-approved drugs, NIH compounds, over-the-counter drugs, off-label prescriptions of anabolic agents, pharmaceutical development-stage compounds, and any combinations thereof. Examples of the anabolic agents include, but are not limited to, vitamin supplements (e.g., but not limited to, Vitamin D), protein or peptide combinations (e.g., but not limited to, whey protein, casein protein, soy protein, egg-white protein, hemp seed, rice protein, pea protein), branched-chain amino acids (e.g., leucine, isoleucine, and valine), glutamine, essential fatty acids (e.g., alpha-linolenic acid and linoleic acid), amino acids, prohormones, creatine, weight loss products, testosterone boosters (e.g., Fenugreek, Eurycoma longifolia, D-aspartic acid, boron, L-carnitine, Tribulus terrestris), synthetic anabolic steroids or anabolic-androgenic steroids (e.g., testosterone propionate, testosterone enanthate, testosterone cypionate, testosterone, nandrolone decanoate, nandrolone phenpropionate, oxandrolone, oxymetholone, methytestosterone, danazol, fluxymesterone, methandrostenolone, boldenone undecylenate, stanozolol, and fluoxymesterone), natural forms of anabolic steroids (e.g., but not limited to, methyltestosterone or android, DHT, and DHEA), growth factors or hormones, IGF1, retinoids, resveratrol, catabolic antagonists, natural or synthetic small molecules, anti-fibrotic compounds, selective androgen receptor modulators (SARMs), anabolic pathway modulators (e.g., 114, 115), and any combinations thereof. In some embodiments, the test compositions can comprise inflammatory activators (e.g., but not limited to, ceramide) to identify anabolic deficiencies.

In some embodiments, the anabolic agents can be pro-anaoblic on muscle. In some embodiments, the anabolic agents can be pro-anabolic on bone.

In some embodiments, the anabolic agents can be ligands that are involved in at least one or more anabolic pathways including, but not limited to, androgen pathway, androgen receptor: testosterone pathway, insulin/IGF-1 pathway, amino acid transport, prostaglandin G-protein coupled, the Activin receptor, the growth hormone pathway, Wnt pathway, calcium pathway, follistatin pathway, adhesion GPCR pathway, myostatin pathway, FGF pathway, NFkB pathway, and any combinations thereof. Most or all of these pathways have ligands that are commercially available as FDA-approved drugs or over-the-counter (OTC) supplements, and can be used as anabolic agents in the assays, methods, systems and/or kits described herein.

For example, in some embodiments, at least one of the anabolic agents can include an amino acid or branched-chain amino acid (e.g., leucine, isoleucine, and valine), including its derivative or variant thereof, that is involved in the amino acid pathway. Amino acids, such as leucine, can activate mTOR through different solute carrier (SLC) family of transporters. Activated mTOR can then induce p70S6k, which can promote protein synthesis. Branched-chain amino acids (BCAA) (e.g., Leucine, isoleucine, valine) and leucine use different SLC transporters. Leucine has been discussed to increase myogenesis.

In some embodiments, at least one of the anabolic agents can include an anabolic steroid and/or an androgen receptor modulator. An example of an anabolic steroid can be testosterone or any derivative or variant thereof that is involved in the androgen receptor (AR): testosterone (T) pathway. An exemplary derivative or variant of the testosterone can be dihydrotestosterone (DHT). Testosterone can stimulate muscle growth in vivo and also in vitro. Muscle precursor stem cells isolated from muscle biopsies display increased myogenic activity when treated with T or DHT. Testosterone can promote growth through multiple pathways. For example, in some embodiments, testosterone can bind androgen receptor (AR), which as a dimer has DNA binding activity. In some embodiments, testosterone can also suppress Redd1, an inducer of a TSC complex that can suppress mTORC1. In some embodiments, testosterone can suppress GSK3, releasing beta-catenin:TCF/LEF driven growth. In some embodiments, testosterone can stimulate Akt, in effect de-repressing mTORC1, through suppression of the TSC complex. The mTORC1 kinase can then induce S6K1, which can promote protein synthesis.

In some embodiments, at least one of the anabolic agents can include a Wnt ligand (e.g., Wnt5a, Wnt5b, and Wnt7a). Wnt ligands (e.g., Wnt5a, Wnt5b, and Wnt7a) can activate Dsh1, a suppressor of GSK3, thereby liberating beta-catenin:TCF/LEF driven growth. In some embodiments, Wnt3a can negatively regulate muscle growth and stimulate fibrosis through upregulation of TGFb. In these embodiments, Wnt3a may not be considered as an anabolic agent.

In some embodiments, at least one of the anabolic agents can include a ligand that activates calcium pathway. Examples of ligands that can activate calcium pathway include, but are not limited to, essential fatty acids (e.g., omega-6 fatty acid), prostaglandin F2a (PGF-2a), interleukin-4 (IL-4). PGF-2a and the calcium activated NFATc pathway can promote muscle fusion and growth. Increases in intracellular calcium can trigger the activation of a phosphatase, calcinuerin. The phosphatase can then dephosphorylate NFAT, which is localized in the cytoplasm. Dephosophorylation of NFAT can then allow for translocation into the nucleus and activation of target genes. Without wishing to be bound by theory, in muscle, the NFATc2 isoform can regulate muscle, as knockout mice have smaller myotubes. A target gene of NFATc2 can be interleukin-4 (IL-4). IL-4 likely plays a director role in muscle growth since antibodies against IL-4 can suppress muscle differentiation, while IL-4 addition to muscle cells from the NFATc2 knockout mouse (which do not express IL-4) can be stimulated to undergo fusion and growth. IL-4 receptor-alpha knockout mice also have defective muscle growth. Prostaglandin F2a can also activate NFATc2 and promote muscle growth, but the pathway for induced muscle growth can be independent of IL-4. Prostaglandins are derived from arachidonic acid, which is an omega-6 fatty acid that is often found in phospholipids of cellular membranes. PGF2a is one of many other fatty acids stimulated by inflammatory conditions. Therefore, regulated inflammation can stimulate muscle growth, while excessive or chronic inflammation can promote catabolism.

In some embodiments, at least one of the anabolic agents can include IGF1 or a derivative or variant thereof. IGF can induce PI3K, which in turn can induce PDK1, which can activate Akt driven suppression of TSC complex, thus promoting mTORC1 activity. Activation of mTORC1 can then induce S6K1, which can promote protein synthesis. IGF1 can be provided by CCR2 recruited macrophages in vivo to stimulate muscle regeneration in response to acute injury.

In some embodiments, at least one of the anabolic agents can include insulin or a derivative or variant thereof. While IGF1 can stimulate muscle synthesis, insulin can inhibit muscle breakdown and promote uptake of amino acids including BCAAs (e.g., Leucine). Without wishing to be bound by theory, insulin appears to operate downstream of mTORC1, in contrast with BCAAs which operate upstream of mTORC1. Anabolic resistance may in part be explained by attenuated response to AAs and reduced insulin sensitivity.

In some embodiments, at least one of the anabolic agents can include follistatin or a derivative or variant thereof. Without wishing to be bound by theory, follistatin can promote growth by competing with the negative regulator Myostatin for binding to the activin receptor IIB (ActRIIB) Follistatin can also likely induce Akt.

In some embodiments, at least one of the anabolic agents can include a peptide hormone that stimulates muscle growth. For example, growth hormone (GH), also called somatotropin, is a peptide hormone that stimulates muscle growth. GH can have direct effects through activation of tyrosine kinase signaling, as well as indirect effects through stimulation of IGF1. In terms of direct effects, the growth hormone receptor can associate with the protein-tyrosine kinase JAK2, which when activated by receptor coupling can stimulate JAK2 phosphorylation of the insulin receptor substrate (IRS) protein, resulting in mTOR kinase activation and subsequent up-regulation of the protein synthesis machinery. Growth hormone can also appear to activate the IGF-independent NFATc2 pathway to stimulate muscle fusion. In terms of indirect effects, growth hormone can stimulate the production of IGF1 in the liver in vivo. There is cross-talk between the insulin, IGF1 and GH pathways.

In some embodiments, at least one of the anabolic agents can include a phospholipid or a derivative or variant thereof that can be recognized by an adhesion G-protein coupled receptor (GPCR) such as BAl1. For example, phosphatidylserine can be an exemplary phospholipid for use as an anabolic agent. Without wishing to be bound by theory, cell death triggered by apoptosis can appear to stimulate myoblast fusion through a phagocytic pathway. Cell death is one outcome of muscle injury and may be a signaling link to promote muscle regeneration. Typically, with cell death, phagocytosis is activated. Phagocytosis a process by which phagocytic cells engulf dead cells. When cells are dying from apoptosis they express a phospholipid, phosphatidylserine (PtSer) on their outer membrane. This phospholipid can be recognized by an adhesion G-protein coupled receptor (GPCR) such as BAI1 initiating a signaling cascade. This signaling cascade can ultimately stimulate myoblast fusion. This stimulation can occur during development, as well as during muscle regeneration and repair. Without wishing to be bound by theory, events in the apopotosis induced myoblast signaling can occur as follows: binding of phosphatidylserine by BAI1 can stimulate the ELMO protein to recruit Dock180 to the plasma membrane. Once at the plasma membrane, complexes of ELMO/Dock180 can stimulate the GTPase protein Rac1. Rac1 activity can then promote phagocytosis, or in the case of myoblasts, can promote fusion of healthy muscle cells. Apoptotic cells can catalyze muscle fusion, but do not appear to directly participate in the muscle fusion process.

In some embodiments, at least one of the anabolic agents can include an antibody or a soluble receptor against myostatin. Myostatin binding to the Act R2b receptor can induce Smad activity, which subsequently inhibit Akt, thereby promoting TSC complex inhibition of mTORC1. Myostatin knockout mice have shown a substantial gain in muscle mass, although there may not be a proportional increase in muscle strength.

In some embodiments, at least one of the anabolic agents can include an anti-inflammatory molecule. Inflammatory cytokines such as TNF-alpha are shown to be elevated in cachexia and can activate NFkB pathway. Activation of NFkB (p50:p65) can induce atrogenes MuRF1 and MAfbx. Activation of NFkB can also result in nuclear translocation of a cytoplasmic precursor. Once in the nucleus, NFkB can activate several target genes, including atrogenes, which can promote muscle breakdown. Accordingly, without wishing to be bound by theory, inhibition of NFkB activation can reduce muscle breakdown.

In some embodiments, at least one of the anabolic agents can include a FGF inhibitor. During aging, FGF2 can increase in muscle stem cells that reside within the muscle stem cell niche, leading to a break in quiescence, eventual loss of self-renewal function and ultimate diminution of the stem cell pool.

The test composition used in the assay, methods, systems and/kits described herein can comprise one or more anabolic agents. In some embodiments, the test composition can comprise more than one (e.g., 2, 3, 4, 5, or more) anabolic agents. In these embodiments, more than one anabolic agents can be included in the test composition to assess any adjuvant effect, which can be additive or synergistic. The term “additive” as used herein in the context of one agent has an additive effect on a second agent, refers to an increase in effectiveness of a first agent in the presence of a second agent as compared to the use of the first agent alone. Stated in another way, the second agent can function as an agent which enhances the physiological response of an organ or organism to the presence of a first agent. Thus, a second agent will increase the effectiveness of the first agent by increasing an individual's response to the presence of the first agent. The term “synergy” or “synergistic” as used herein refers to the interaction of two or more agents so that their combined effect is greater than each of their individual effects at the same dose alone.

In some embodiments, the anabolic agent(s) included in the test composition can include a protein, peptide, nucleic acid (e.g., but not limited to DNA, RNA, shRNA, siRNA, miRNA, and modified RNA), aptamer, antibody or a portion thereof, antibody-like molecule, small molecule, or any combination thereof.

In some embodiments, the anabolic agent(s) included in the test composition can include a known therapeutic agent or a candidate agent for anabolic treatment (e.g., reducing muscle and/or bone loss; and/or inducing muscle and/or bone growth). For example, the anabolic agents included in the test composition can encompass FDA-approved compounds, natural-like molecules or synthetic small molecules from various chemical libraries.

In some embodiments, the anabolic agent(s) included in the test composition can include one or more compounds as shown in FIG. 2B. In some embodiments, the anabolic agent(s) included in the test composition can include one or more compound classes as shown in FIG. 2B.

In some embodiments, a test composition can comprise at least one agent that has been indicated for stimulating muscle and/or bone growth, e.g., a known anabolic agent such as acidic and basic fibroblast growth factors (aFGF and bFGF); epidermal growth factor (EGF); insulin-like growth factor-1 (IGF-1); platelet derived growth factor (PDGF); transforming growth factor β or α (TGF-β or TGF-α); and nerve growth factor (NGF); an agent commonly administered for treatment of a musculoskeletal disease or disorder, e.g., non-steroidal anti-inflammatory drugs (NSAIDs) such as indomethacin, ketoprofen, ibuprofen, acetylsalicylic acid (ASA), and flurbiprofen, local analgesic therapies, corticosteroid; immunosuppressors (e.g., but not limited to, rapamycin and FK-506); nutritional supplements and anabolic supplements such as testosterone and analogs thereof (e.g., DHT), growth hormone and analog thereof, vitamin D, bisphosphonates, bone resorption antagonists (e.g., denosumab), and any art-recognized agents for treatment of a musculoskeletal disease or disorder, e.g., the ones described in U.S. Pat. App. No. US 2007/0213308, and US 2008/0119426, and U.S. Pat. No. 7,897,588, the contents of which are incorporated herein by reference.

One embodiment of a panel of the test compositions used to produce optimized and personalized or stratified anabolic profiles in one or more embodiments of the assays, methods, systems and/or kits described herein is shown in Table 1 below. This exemplary panel of the test compositions is provided as an illustrative example and is not construed to be limiting. In some embodiments, the panel of the test compositions used in the assays, methods, systems and/or kits described herein can comprise at least one or any combinations of the following classes, including, but not limited to, growth factors or hormones (e.g., but not limited to molecules 1-12 in Table 1), synthetic anabolic steroids (e.g., but not limited to molecules 14-32 in Table 1), natural anabolic steroids (e.g., but not limited to molecules 33-35 in Table 1), catabolic antagonists (e.g., but not limited to molecules 36-44, 56-58 in Table 1), vitamins (e.g., but not limited to molecule 45 in Table 1), synthetic or natural small molecules (e.g., but not limited to, molecules 46-48, and 114 in Table 1), amino acids, protein or peptide combinations (e.g., but not limited to, molecules 49-55 in Table 1), anti-fibrotic molecules (e.g., but not limited to molecule 61 in Table 1), selective androgen receptor modulators (SARMs) (e.g., but not limited to molecules 70-77 in Table 1), inflammatory activators to identify deficiencies (e.g., but not limited to molecules 81 and 83 in Table 1), other inhibitors (e.g., but not limited to, molecules 108-109 in Table 1), known or potential anabolic pathway modulators (e.g., but not limited to, molecules 112-113 in Table 1).

TABLE 1
An exemplary panel of test compositions used
in one or more embodiments of the assay, method,
system and/or kit described herein.

| | |
|---|---|
| 1 | Insulin/Slin |
| 2 | HGH/Somatotropin, |
| 3 | HGH/Somatotropin derivatives |
| 4 | GHRP-2 |
| 5 | GHRP-6 |
| 6 | hexarelin |
| 7 | HGF Frag 176-191 |
| 8 | IGF DES(1-3) |
| 9 | IGF-1 LR3 |
| 10 | MGF |
| 11 | Modified GRF 1-29 |
| 12 | PEG-MGF |
| 13 | oxymetholone/Anadrol-50 |
| 14 | Boldenone (Equipoise/EQ) |
| 15 | Methenolone (Primobolan) |
| 16 | Drostanolone (Masteron) |
| 17 | Testosterone blend (Sustanon/Omnadren) |
| 18 | Testosterone enanthate |
| 19 | Methandrostenolone (Dianabol/D-bol) |
| 20 | Chlorodehydromethyl testosterone (Turinabol) |
| 21 | Oxymetholone/Anadrol-50 |
| 22 | Nandrol/nandrolone |
| 23 | decanoate/deca-durabolin/deca, |
| 24 | oxandrin/oxandralone/anavar, |
| 25 | danazol, |
| 26 | trenbolone acetate/Tren A/Fina, |
| 27 | Sustanon5000/250/mixed |
| 28 | testo |
| | propionate/phenylpropionate/isocaproate/decanoate, |
| 29 | testo/testosterone cypionate, |
| 30 | testoterone/androgel |
| 31 | stanozolol/Winstrol/Winny/Win-V, |
| 32 | Fluoxymesterone (Halotestin) |
| 33 | Methyltestosterone/Android |
| 34 | DHT |
| 35 | DHEA |
| 36 | Rapamycin |
| 37 | FK506 |
| 38 | Pentoxyphilin |
| 39 | Thalidomoide |
| 40 | GSH/Glutathione |
| 41 | NAC |
| 42 | NSAIDs |
| 43 | EPA |
| 44 | JQ1 |
| 45 | vitamins (e.g., Vitamin D) |
| 46 | creatine, |
| 47 | L-carnitine, |
| 48 | L-glutamine |
| 49 | Protein powder (whey, egg, soy, rice) |
| 50 | EAA/9, |
| 51 | BCAA/Leucine + Isoleucine + Valine), |
| 52 | Leucine, |
| 53 | Leucine metabolite HMB, |
| 54 | Arginine |
| 55 | RGD peptide: Cyclo [Arg-Gly-Asp-D-Phe-Val] |
| 56 | Follistatin |
| 57 | Follistatin 344 |
| 58 | FLRG-1 (Follistatin related gene) |
| 59 | Erythropoietin |
| 60 | CRH |
| 61 | Losartan |
| 62 | calstabin1/2 |
| 63 | Halofuginone |
| 64 | Clenbuterol |
| 65 | tibolone |
| 66 | Des-Acyl Ghrelin |
| 67 | ghrelin |
| 68 | zilpaterol |
| 69 | Resveratrol |
| 70 | Andarine, |
| 71 | Ostarine, |
| 72 | AC-262356 |
| 73 | BMS-564929 |
| 74 | LGD-4033, LGD-2226, LGD-2941 |
| 75 | propionalide, |
| 76 | S-40542, |
| 77 | MK-0773 |
| 78 | 10-Hydroxycamptothecin |
| 79 | AG-1296 |
| 80 | Tyrosine kinase inhibitor: Beclomethasone |
| 81 | Dihydroceramide(prodrug)/Ceramide |
| 82 | CMLD004378 |
| 83 | Ebselen |
| 84 | CDK/GSK3b inhibitor: Indirubin, Indirubin-3′- |
| | monoxime |
| 85 | GASP-1 (GDF assoc serum protein) |
| 86 | Mycophenolic acid/Mycophenolate mofetil(prodrug) |
| 87 | Parthenolide |
| 88 | Prostaglandin F2alpha (PGF2a), 17-phPGF2a,17- |
| | phenyl trinor PGF2a |
| 89 | Retinoids (1st gen: Retinoic acid/13-cis Retinoic |
| | acid/9-cis Retinoic acid; 2nd gen: Etretinate; 3rd |
| | gen: Bexarotene; synthetic: 4- |
| | Hydroxyphenylretinamide; Derivatives: AM-580; |
| | TTNPB) |
| 90 | ROLIPRAM |
| 91 | Forskolin |
| 92 | phytoandrogens (daidzein, gutta-percha |
| | triterpenoids) |
| 93 | Xenoandrogens (modified tocopherols, modified |
| | nicotinamide) |
| 94 | Phytoecdysteroids (25S)-20,22-0-(R- |
| | ethylidene)inokosterone |
| 95 | ursolic acid |
| 96 | Wnt7a |
| 97 | Clemastine fumarate |
| 98 | Suramin sodium |
| 99 | Procainamide |
| 100 | Fluphenazine 2HCl |
| 101 | Mevastatin |
| 102 | Sotalol HCl/formoterol/salmeterol |
| 103 | Ipamorelin |
| 104 | CJC-1295 |
| 105 | Thymosin beta 4 (TB-500) |
| 106 | Sermorelin |
| 107 | CoQ |
| 108 | Activin A |
| 109 | MSTN |
| 110 | IL-4 |
| 111 | Calcium |
| 112 | PtdSer |
| 113 | zVAD |
| 114 | Arachidonic Acid (ARA) |

Due to an increasingly diverse population showing different anabolic responsiveness, a panel of anabolic agents can be selected for pharmacologic diversity in order to achieve a broad anabolic landscape, and thus provide a diagnostic tool to gauge therapeutic effectiveness by measuring anabolic activity in muscle cells or muscle stem cells based on an in vitro cell-based assay described herein. By identifying patient subgroups who are more likely to benefit from a particular treatment or by identifying a personalized treatment for a specific subject, muscle anabolic diagnostics described herein can provide treatment options as decision support to maintain muscle mass and to counter muscle loss, thus reducing risk for functional decline.

One skilled in the art would be able to readily determine recommended dosage levels for the selected test composition and/or anabolic agents included therein, e.g., based on the anabolic response of the musculoskeletal cells or precursor cells thereof to different concentrations of the anabolic agents used in one or more embodiments of the assay described herein, and/or by consulting appropriate references such as drug package inserts, FDA guidelines, and the Physician's Desk Reference. One of skill in the art can readily adjust dosage, depending on a number of factors such as types and/or potency of anabolic agents, severity of a musculoskeletal disease or disorder, physical condition of a subject (e.g., ages, genders, and weights), administration routes, other medications taken by a subject, and any combinations thereof.

In some embodiments, the test compositions can be present in a growth medium or differentiation medium added to the musculoskeletal cells or precursor cells thereof at a concentration of about 1 nM to about 500 μM, about 10 nM to about 400 μM, about 100 nM to about 300 μM, about 1 μM to about 200 μM, about 5 μM to about 100 μM. In some embodiments, the test compositions can be present in a growth medium or differentiation medium added to the musculoskeletal cells or precursor cells thereof at a concentration of at least about 1 μM, at least about 5 μM, at least about 10 μM, at least about 25 μM, at least about 50 μM or more.

Pharmaceutical Compositions for Treatment/Prevention of a Musculoskeletal Disease or Disorder, or for Muscle Augmentation or Mitigation of Muscle and/or Bone Loss

For in vivo administration to subjects with the selected test composition or anabolic agent/composition for treatment/prevention of a musculoskeletal disease or disorder, or for muscle augmentation or mitigation of muscle and/or bone loss, one aspect provided herein relates to pharmaceutical compositions comprising a therapeutically effective amount of a selected test composition or anabolic agent/composition (based on its ranking in the assay described herein) or a pharmaceutically acceptable salt thereof, and a pharmaceutically acceptable carrier.

In various embodiments, the therapeutically effective amount of a selected test composition and/or anabolic agent is sufficient to decrease at least one symptom of a musculoskeletal disease or disorder (e.g., muscle wasting/bone loss) by at least about 10%, at least about 15%, at least about 20%, at least about 30%, at least about 40%, or at least about 50%. In another embodiment, the therapeutically effective amount of a selected test composition and/or anabolic agent is sufficient to decrease at least one symptom of a musculoskeletal disease or disorder (e.g., muscle wasting/bone loss) by more than 50%, e.g., at least about 60%, or at least about 70%. In one embodiment, the therapeutically effective amount of a selected test composition and/or anabolic agent is sufficient to decrease at least one symptom of a musculoskeletal disease or disorder (e.g., muscle wasting/bone loss) by at least about 80%, at least about 90% or greater, as compared to a control (e.g., a subject having the same condition as the treated subject is administered without the test composition, or a subject whose anabolic profile does not recommend the test composition is administered with the test composition). In one embodiment, the therapeutically effective amount of a selected test composition and/or anabolic agent is sufficient to decrease at least one marker associated with a musculoskeletal disease or disorder (e.g., creatine kinase) by at least about 10%, at least about 15%, at least about 20%, at least about 30%, at least about 40%, or at least about 50%. In another embodiment, the therapeutically effective amount of a selected test composition and/or anabolic agent is sufficient to decrease at least one marker associated with a musculoskeletal disease or disorder (e.g., creatine kinase) by more than 50%, e.g., at least about 60%, or at least about 70%. In one embodiment, the therapeutically effective amount of a selected test composition and/or anabolic agent is sufficient to decrease at least one marker associated with a musculoskeletal disease or disorder (e.g., creatine kinase) by at least about 80%, at least about 90% or greater, as compared to a control (e.g., a subject having the same condition as the treated subject is administered without the test composition, or a subject whose anabolic profile does not recommend the test composition is administered with the test composition).

In various embodiments, the therapeutically effective amount of a selected test composition and/or anabolic agent is sufficient to increase muscle and/or bone mass by at least about 10%, at least about 15%, at least about 20%, at least about 30%, at least about 40%, or at least about 50%. In another embodiment, the therapeutically effective amount of a selected test composition and/or anabolic agent is sufficient to increase muscle and/or bone mass by more than 50%, e.g., at least about 60%, or at least about 70%. In one embodiment, the therapeutically effective amount of a selected test composition and/or anabolic agent is sufficient to increase muscle and/or bone mass by at least about 80%, at least about 90% or greater, as compared to a control (e.g., a subject having the same condition as the treated subject is administered without the test composition, or a subject whose anabolic profile does not recommend the test composition is administered with the test composition).

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (i) sugars, such as lactose, glucose and sucrose; (ii) starches, such as corn starch and potato starch; (iii) cellulose, and its derivatives, such as sodium carboxymethyl cellulose, methylcellulose, ethyl cellulose, microcrystalline cellulose and cellulose acetate; (iv) powdered tragacanth; (v) malt; (vi) gelatin; (vii) lubricating agents, such as magnesium stearate, sodium lauryl sulfate and talc; (viii) excipients, such as cocoa butter and suppository waxes; (ix) oils, such as peanut oil, cottonseed oil, safflower oil, sesame oil, olive oil, corn oil and soybean oil; (x) glycols, such as propylene glycol; (xi) polyols, such as glycerin, sorbitol, mannitol and polyethylene glycol (PEG); (xii) esters, such as ethyl oleate and ethyl laurate; (xiii) agar; (xiv) buffering agents, such as magnesium hydroxide and aluminum hydroxide; (xv) alginic acid; (xvi) pyrogen-free water; (xvii) isotonic saline; (xviii) Ringer's solution; (xix) ethyl alcohol; (xx) pH buffered solutions; (xxi) polyesters, polycarbonates and/or polyanhydrides; (xxii) bulking agents, such as polypeptides and amino acids (xxiii) serum component, such as serum albumin, HDL and LDL; (xxiv) C2-C12 alchols, such as ethanol; and (xxv) other non-toxic compatible substances employed in pharmaceutical formulations. Wetting agents, coloring agents, release agents, coating agents, sweetening agents, flavoring agents, perfuming agents, preservative and antioxidants can also be present in the formulation.

Pharmaceutically acceptable carriers can vary in a composition of the invention, depending on the administration route and formulation. For example, the pharmaceutically acceptable composition of the invention can be delivered via injection. These routes for administration (delivery) include, but are not limited to, subcutaneous or parenteral including intravenous, intraarterial, intramuscular, intraperitoneal, intramyocardial, and infusion techniques. In one embodiment, the pharmaceutical acceptable composition is in a form that is suitable for injection. In another embodiment, the pharmaceutical composition is formulated for delivery by a catheter.

When administering a pharmaceutical composition of the invention parenterally, it will be generally formulated in a unit dosage injectable form (solution, suspension, emulsion). The pharmaceutical formulations suitable for injection include sterile aqueous solutions or dispersions. The carrier can be a solvent or dispersing medium containing, for example, water, cell culture medium, buffers (e.g., phosphate buffered saline), polyol (for example, glycerol, propylene glycol, liquid polyethylene glycol, and the like), suitable mixtures thereof. In some embodiments, the pharmaceutical carrier can be a buffered solution (e.g. PBS).

In some embodiments, the pharmaceutical composition can be formulated in an emulsion or a gel.

In some embodiments, the pharmaceutical compositions described herein can be formulated for oral administration or for inhalation. For oral administration, suitable dosage forms can include tablets, troches, cachets, caplets, and capsules, including hard and soft gelatin capsules.

In some embodiments, the pharmaceutical compositions can be formulated for sustained release. In some embodiments, the pharmaceutical compositions can be formulated in controlled-release drug-delivery systems.

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it may be desirable to include isotonic agents, for example, sugars, sodium chloride, and the like.

The compositions can also contain auxiliary substances such as wetting or emulsifying agents, pH buffering agents, gelling or viscosity enhancing additives, preservatives, colors, binders, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation. With respect to compositions of the invention, however, any vehicle, diluent, or additive used should have to be biocompatible with the selected test composition and/or anabolic agents.

The pharmaceutical compositions can be isotonic, i.e., they can have the same osmotic pressure as blood and lacrimal fluid. The desired isotonicity of the compositions of the invention can be accomplished using sodium chloride, or other pharmaceutically acceptable agents such as dextrose, boric acid, sodium tartrate, propylene glycol or other inorganic or organic solutes. In one embodiment, sodium chloride is used in buffers containing sodium ions.

Viscosity of the compositions can be maintained at the selected level using a pharmaceutically acceptable thickening agent. In one embodiment, methylcellulose is used because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount which will achieve the selected viscosity. Viscous compositions are normally prepared from solutions by the addition of such thickening agents.

Typically, any additives (in addition to the selected test composition and/or anabolic agents) can be present in an amount of 0.001 to 50 wt % solution, e.g., in a buffered solution (e.g., phosphate buffered saline), and the active ingredient is present in the order of micrograms to milligrams to grams, such as about 0.0001 to about 5 wt %, about 0.0001 to about 1 wt %, about 0.0001 to about 0.05 wt % or about 0.001 to about 20 wt %, about 0.01 to about 10 wt %, and about 0.05 to about 5 wt %. For any therapeutic composition to be administered to a subject in need thereof, and for any particular method of administration, it is preferred to determine toxicity, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan.

Those skilled in the art will recognize that the components of the compositions should be selected to be biocompatible with respect to the active agent, e.g., the selected test composition and/or anabolic agents. This will present no problem to those skilled in chemical and pharmaceutical principles, or problems can be readily avoided by reference to standard texts or by simple experiments (not involving undue experimentation).

The compositions of the invention can be prepared by mixing the ingredients following generally-accepted procedures. For example, the ingredients can be mixed in an appropriate pharmaceutically acceptable carrier and the mixture can be adjusted to the final concentration and viscosity by the addition of water or thickening agent and possibly a buffer to control pH or an additional solute to control tonicity. Generally the pH can vary from about 3 to about 7.5. In some embodiments, the pH of the composition can be about 6.5 to about 7.5. Compositions can be administered in dosages and by techniques well known to those skilled in the medical and veterinary arts taking into consideration such factors as the age, sex, weight, and condition of the particular patient, and the composition form used for administration (e.g., liquid). Dosages for humans or other mammals can be determined without undue experimentation by a skilled artisan.

### Systems and Computer Readable Media

A further aspect provided herein relates to systems (and computer readable physical storage media for causing computer systems) to perform one or more embodiments of the assay described herein, e.g., for profiling anabolic responses of a subject or a population subgroup to a plurality of test compositions, and/or for optimizing or selecting a treatment regimen for a subject with a musculoskeletal disease or disorder based on the subject-specific (personalized) or subject-matched (stratification) anabolic profile.

A system for generating anabolic profiles for at least one or more subjects is provided herein. The system comprises a determination module configured to receive at least one or more samples each comprising a population of the musculoskeletal cells or precursor cells thereof, and subject the cells to at least one analysis or at least two analyses to quantify at least one or more anabolic responses (e.g., muscle and/or bone cell proliferation and/or differentiation) of the cells in response to test compositions exposed to the cells.

The system can further comprise a computer system, the computer system including a processor and associated memory including instructions that, when executed by the processor, cause the processor to control operation of the determination module to perform that least one analysis on one or more samples.

In some embodiments, before the musculoskeletal cells or precursor cells thereof are received in the determination module, the cells can have been placed in an assay container (e.g., a micro-titer plate) and contacted with a plurality of test compositions described herein. Accordingly, in these embodiments, the determination module is configured to receive, e.g., a plate of musculoskeletal cells or precursor cells thereof treated with different test compositions.

In other embodiments, the determination module can be configured to contact the cells with a plurality of test compositions described herein before subjecting them to the analyses. Accordingly, in some embodiments, the determination module can be configured to receive at least one or more samples each comprising a population of the musculoskeletal cells or precursor cells thereof and perform the following steps: (i) contacting the musculoskeletal cells or precursor cells thereof with a plurality of test compositions each comprising at least one agent selected to increase and/or maintain muscle and/or bone growth; and (ii) subjecting the musculoskeletal cells or precursor cells thereof to at least one analysis (including, e.g., at least two analyses) to quantify muscle growth and/or bone growth of the musculoskeletal cells or precursor cells thereof in response to the test compositions;

A sample received by the determination module can contain musculoskeletal cells or precursor cells thereof obtained or derived from a biological sample (e.g., a muscle biopsy or a blood sample) of a subject who is seeking an anabolic treatment. Alternatively, a sample can contain musculoskeletal cells or precursor cells thereof obtained or derived from a panel of tissue specimens or cells representing one or more different population subgroups. The panel of tissue specimens or cells representing one or more different population subgroups can be obtained from a tissue or cell depository. In some embodiments, the musculoskeletal cells or precursor cells thereof can contain cells from individuals that share at least one feature such as a phenotypic feature (e.g., but not limited to, age, gender, BMI, condition, and ethnicity). In some embodiments, a sample can contain musculoskeletal cells or precursor cells thereof obtained or derived from a subject who is determined to have or have a risk for a musculoskeletal disease or disorder described herein.

The determination module can be configured in any manner to accommodate different types of analyses selected to quantify muscle growth and/or bone growth of the musculoskeletal or precursor cells thereof. In some embodiments, the determination module can be configured to determine the number of multi-nucleated cells formed by fusion of mononucleated musculoskeletal cells or precursor cells thereof for quantifying muscle growth. For example, the determination module can be configured to include a microscope and an imaging system that permit examining and/or capturing images of the phenotypes and/or morphology of the musculoskeletal cells or precursor cells thereof for muscle growth analysis (e.g., quantifying formation of multi-nucleated cells and/or fusion of mononucleated muscle cells).

In some embodiments, the determination module can be further configured to determine the number of bone cells differentiated from the musculoskeletal cells or precursor cells thereof (e.g., muscle cells or bone precursor cells) for quantifying bone growth. By way of example only, the determination module can be configured to perform immunostaining, protein expression analysis, and/or nucleic acid expression analysis on the cells, e.g., to detect the bone cells based on expression of a bone marker. In one embodiment, the bone marker is alkaline phosphatase (ALP). Other examples of a bone marker can include, but are not limited to type I collagen propetides and/or osetocalcin. The images and/or data collected by the determination module can be stored in the storage device for subsequent analyses.

In one embodiment, bone cell proliferation and/or differentiation can be quantified by imaging the cells after they are stained for a bone cell marker, e.g., alkaline phosphatase (ALP), to determine the number of cells that express a bone marker. In such embodiments, the determination module can be configured to include an automated immunohistochemistry apparatus that performs cell immunostaining Examples of such automated immunohistochemistry apparatus are commercially available, for example such Autostainers 360, 480, 720 and Labvision PT module machines from LabVision Corporation, which are disclosed in U.S. Pat. Nos. 7,435,383; 6,998,270; 6,746,851, 6,735,531; 6,349,264; and 5,839; 091 which are incorporated herein in their entirety by reference. Other commercially available automated immunohistochemistry instruments are also encompassed for use in the present invention, for example, but not are limited BOND™ Automated Immunohistochemistry & In Situ Hybridization System, Automate slide loader from GTI vision. Automated analysis of immunohistochemistry can be performed by commercially available systems such as, for example, IHC Scorer and Path EX, which can be combined with the Applied spectral Images (ASI) CytoLab view, also available from GTI vision or Applied Spectral Imaging (ASI) which can all be integrated into data sharing systems such as, for example, Laboratory Information System (LIS), which incorporates Picture Archive Communication System (PACS), also available from Applied Spectral Imaging (ASI) (see world-wide-web: spectral-imaging.com). Other a determination module can be an automated immunohistochemistry systems such as NexES® automated immunohistochemistry (IHC) slide staining system or BenchMark® LT automated IHC instrument from Ventana Discovery SA, which can be combined with VIAS™ image analysis system also available Ventana Discovery. BioGenex Super Sensitive MultiLink® Detection Systems, in either manual or automated protocols can also be used as the determination module, e.g., using the BioGenex Automated Staining Systems. Such systems can be combined with a BioGenex automated staining systems, the i6000™ (and its predecessor, the OptiMax® Plus), which is geared for the Clinical Diagnostics lab, and the GenoMx 6000™, for Drug Discovery labs. Both systems BioGenex systems perform “All-in-One, All-at-Once” functions for cell and tissue testing, such as Immunohistochemistry (IHC) and In Situ Hybridization (ISH).

In other embodiments, other non-imaging or more quantitative methods, e.g., but not limited to, polymerase chain reaction (PCR) method, Western blot, and ELISA, can be used to determine the presence or absence of muscle or bone cell proliferation and/or differentiation, e.g., based on expression of a muscle or bone marker. In these embodiments, the determination module can further comprise an amplification device (e.g., a PCR machine), a robotic module (e.g., to perform transfer of a sample from one chamber to another, and/or to add a reagent to a sample), a signal detection device (e.g., a spectrophotometer), and any combinations thereof. Exemplary systems for automated protein expression analysis of a specific muscle and/or bone marker, can include, for example, but not limited to, Mass Spectrometry systems including MALDI-TOF, or Matrix Assisted Laser Desorption Ionization—Time of Flight systems; SELDI-TOF-MS ProteinChip array profiling systems, e.g. Machines with Ciphergen Protein Biology System II™ software; systems for analyzing gene expression data (see for example U.S. 2003/0194711); systems for array based expression analysis, for example HT array systems and cartridge array systems available from Affymetrix (Santa Clara, Calif. 95051) AutoLoader, Complete GeneChip® Instrument System, Fluidics Station 450, Hybridization Oven 645, QC Toolbox Software Kit, Scanner 3000 7G, Scanner 3000 7G plus Targeted Genotyping System, Scanner 3000 7G Whole-Genome Association System, GeneTitan™ Instrument, GeneChip® Array Station, HT Array; an automated ELISA system (e.g. DSX® or DS2® form Dynax, Chantilly, Va. or the ENEASYSTEM III®, Triturus®, The Mago® Plus); Densitometers (e.g. X-Rite-508-Spectro Densitometer®, The HYRYS™ 2 densitometer); automated Fluorescence in situ hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, Becton Dickinson); radio isotope analyzers (e.g. scintillation counters).

Alternatively, determination modules 40 for determining the presence or absence of muscle or bone cell proliferation and/or differentiation, e.g., based on expression of a muscle or bone marker may include known systems for automated detection of nucleotide sequences (i.e. RNA expression) of corresponding muscle and/or bone markers, including sequence analysis including but not limited to Hitachi FMBIO® and Hitachi FMBIO® II Fluorescent Scanners (available from Hitachi Genetic Systems, Alameda, Calif.); Spectrumedix® SCE 9610 Fully Automated 96-Capillary Electrophoresis Genetic Analysis Systems (available from SpectruMedix LLC, State College, Pa.); ABI PRISM® 377 DNA Sequencer, ABI® 373 DNA Sequencer, ABI PRISM® 310 Genetic Analyzer, ABI PRISM® 3100 Genetic Analyzer, and ABI PRISM® 3700 DNA Analyzer (available from Applied Biosystems, Foster City, Calif.); Molecular Dynamics Fluorlmager™ 575, SI Fluorescent Scanners, and Molecular Dynamics Fluorlmager™ 595 Fluorescent Scanners (available from Amersham Biosciences UK Limited, Little Chalfont, Buckinghamshire, England); GenomyxSC™ DNA Sequencing System (available from Genomyx Corporation (Foster City, Calif.); and Pharmacia ALF™ DNA Sequencer and Pharmacia ALFexpress™ (available from Amersham Biosciences UK Limited, Little Chalfont, Buckinghamshire, England).

Embodiments of the system described herein also comprises a storage device configured to store data output from the determination module; and a display module for displaying a content based in part on the data output from said determination module and/or an analysis module. The content displayed in the display module can comprise a signal indicative of a partial or entire ranking of the anabolic efficacy of the test compositions, or a signal indicative of at least one test composition recommended for the subject's treatment, or a signal indicative of no test composition recommended for the subject.

The storage device can be separated from the computer system and/or located remotely over a network. In these embodiments, the computer system can control a remote system to access data stored in the storage device and/or save data to the storage device. Alternatively or additionally, the storage device can be integrated within the local computer system.

In some embodiments, the system and/or computer system can further comprise an analysis module configured to rank anabolic efficacy of the test compositions as a function of the data output from said determination module.

In some embodiments, the analysis module can comprise at least one image analysis algorithm to quantify muscle growth and/or bone growth based on the images of cells captured by the determination module and stored in the storage device. The image analysis algorithm can be programmed to quantify the number of multi-nucleated cells formed by fusion of mononucleated musculoskeletal cells or precursor cells thereof in each image and compute the corresponding fusion index or fusion distribution as described above. Alternatively or additionally, the image analysis algorithm can be programmed to quantify the number of bone cells present in each image, e.g., based on expression of a bone marker described herein.

In some embodiments, the analysis module can further comprise a comparison algorithm adapted to compare the data output from the determination module with reference data stored on the storage device. The reference data can include anabolic data (e.g., muscle and/or bone growth) from a negative control (e.g., in the absence of the test composition(s)), anabolic data (e.g., muscle and/or bone growth) from a positive control (e.g., in the presence of an anabolic agent that is known to stimulate muscle and/or bone cell proliferation and/or differentiation), anabolic data (e.g., muscle and/or bone growth) of one or more subjects from at least one previous time point; and/or anabolic data (e.g., muscle and/or bone growth) of one or more normal healthy subjects without any known muscle or bone loss.

A computer readable physical storage medium having computer readable instructions recorded thereon to define software modules for implementing a method on a computer is also described herein. The computer readable storage medium comprises: (a) instructions for analyzing the data stored on a storage device that in part comprises data indicative of anabolic responses of musculoskeletal cells or precursor cells thereof to a plurality of test compositions comprising at least one agent selected to maintain and/or increase at least muscle or bone growth; wherein the data analysis ranks anabolic efficacy of the test compositions based on the data stored on the storage device; and (b) instructions for displaying a content based in part on the data stored on the storage device. In some embodiments, the content to be displayed can comprise a signal indicative of at least a partial ranking of the anabolic efficacy of the test compositions. In some embodiments, the content to be displayed can comprise a signal indicative of at least one test composition recommended for the subject's treatment. In other embodiments, the content to be displayed can comprise a signal indicative of no test composition recommended for the subject.

Embodiments of the systems have been described through functional modules, which are defined by computer executable instructions recorded on computer readable media and which cause a computer to perform method steps when executed. The modules have been segregated by function for the sake of clarity. However, it should be understood that the modules need not correspond to discrete blocks of code and the described functions can be carried out by the execution of various code portions stored on various media and executed at various times. Furthermore, it should be appreciated that the modules may perform other functions, thus the modules are not limited to having any particular functions or set of functions.

The computer readable media can be any available tangible, non-transitory media that can be accessed by a computer. Computer readable media includes volatile and nonvolatile, removable and non-removable tangible media implemented in any method or technology for storage of information such as computer readable instructions, data structures, program modules or other data. Computer readable media includes, but is not limited to, RAM (random access memory), ROM (read only memory), EPROM (eraseable programmable read only memory), EEPROM (electrically eraseable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

In some embodiments, the computer readable storage media 200 can include the “cloud” system, in which a user can store data on a remote server, and later access the data or perform further analysis of the data from the remote server. For example, the database comprising anabolic information for a plurality of population subgroups stratified or characterized by at least one feature such as phenotypic feature as described herein can be stored in the “cloud” system, which can be later retrieved to a computer system or other processing device such as a tablet PC or mobile phone in accordance with the instructions contained in the computer readable storage media, wherein the anabolic information for each of the population subgroups can comprise rankings of a plurality of anabolic agents based on their anabolic efficacy in each of the population subgroups. By way of example only, in a health food store, a customer can use such stratification profile database to choose an anabolic/nutritional supplement, for example, by matching his/her phenotypic profile (e.g., age, gender, ethnicity, condition, and/or BMI) to one of the population subgroups stored in the database. Based on the anabolic profile of the associated population subgroup, the corresponding anabolic/nutritional supplement can then be recommended for the customer. In one embodiment, the anabolic/nutritional supplement can be recommended based on the customer's age and gender. Alternatively, a mobile phone application program can be developed to permit access of the stratification profile database and to output a recommended anabolic product based on the input of the subject's information such as at least one phenotypic feature, including, but not limited to, age, gender, ethnicity, condition, and/or BMI, and association of the subject's information to one of the population subgroups.

Computer-readable data embodied on one or more computer-readable media, or computer readable medium 200, may define instructions, for example, as part of one or more programs, that, as a result of being executed by a computer, instruct the computer to perform one or more of the functions described herein (e.g., in relation to system 10, or computer readable medium 200), and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of system 10, or computer readable medium 200 described herein, may be distributed across one or more of such components, and may be in transition there between.

The computer-readable media can be transportable such that the instructions stored thereon can be loaded onto any computer resource to implement various aspects described herein. In addition, it should be appreciated that the instructions stored on the computer readable media, or computer-readable medium 200, described above, are not limited to instructions embodied as part of an application program running on a host computer. Rather, the instructions may be embodied as any type of computer code (e.g., software or microcode) that can be employed to program a computer to implement various aspects described herein. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments described herein can include a determination module, a storage device, and a display module. In some embodiments, certain embodiments described herein can further include an analysis module. The functional modules can be executed on one, or multiple, computers, or by using one, or multiple, computer networks. The determination module 40 has computer executable instructions to provide sequence information in computer readable form.

Information about anabolic responses (e.g., muscle and/or bone cell proliferation and/or differentiation) of cells to a plurality of test compositions determined in the determination module can be read by the storage device. As used herein the “storage device” 30 is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage devices 30 also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device 30 is adapted or configured for having recorded thereon information about anabolic responses. Such information may be provided in digital form that can be transmitted and read electronically, e.g., via the Internet, on diskette, via USB (universal serial bus) or via any other suitable mode of communication, e.g., the “cloud”.

As used herein, “information about anabolic responses of cells” refers to any information about muscle cell proliferation, growth and/or differentiation, bone cell proliferation, growth and/or differentiation, or both, including but not limited to images and scoring indices/indicators of cell morphology/phenotype of muscle cells and/or bone cells, information related to expression of at least one muscle cell-marker or at least one bone cell-specific marker in the cells (e.g., in protein level or in mRNA level), information related to specific molecules (e.g., cytokines, extracellular matrix molecules, growth factors, MMPs) secreted by muscle cells or bone cells, and any combinations thereof. Moreover, information “related to” anabolic responses of cells includes functions of muscle cells (e.g., contractility) or bone cells, detection of the presence or absence of a muscle- or bone-specific marker or specific molecules secreted by muscle cells or bone cells (e.g., presence or absence of an amino acid sequence, nucleotide sequence, or post translational modification), determination of the concentration of a muscle- or bone-specific marker or specific molecules secreted by muscle cells or bone cells in the sample (e.g., amino acid sequence levels, or nucleotide (RNA or DNA) expression levels, or level of post translational modification), and the like. In some embodiments, the information related to anabolic responses also includes arithmetic manipulation of expression levels of at least two or more specific cell markers.

As used herein, “stored” refers to a process for encoding information on the storage device 30. Those skilled in the art can readily adopt any of the presently known methods for recording information on known media to generate manufactures comprising the information of anabolic responses.

A variety of software programs and formats can be used to store the information of anabolic responses on the storage device. Any number of data processor structuring formats (e.g., text file or database) can be employed to obtain or create a medium having recorded thereon the information of anabolic responses.

By providing in computer-readable form information about anabolic responses of cells to a plurality of test compositions, one can use the information in the readable form in the analysis module 80 to rank anabolic efficacy of the test compositions, e.g., based on the ability of each individual test composition to stimulate muscle and/or bone cell proliferation and/or differentiation. For example, in the analysis of muscle cell proliferation and/or differentiation, images of the cells treated with various test compositions can be analyzed, e.g., with imaging analysis programs such as ImageJ or MATHLAB, for the presence or absence of multi-nucleated muscle cells (e.g., more than 2 nuclei in a cell) formed from mononucleated cells. The analysis module can assign a fusion or anabolic efficacy index to each test composition, e.g., based on the number of multi-nucleated cells (e.g., more than 2 nuclei in a cell). In some embodiments, a fusion or anabolic efficacy index is a ratio of the number of nuclei involved in cells with at least 2 nuclei to total number of nuclei of all the cells. For example, as shown in FIG. 2A, a higher fusion or anabolic efficacy index assigned to a test composition indicates that the test composition is capable of inducing a larger fraction of mononucleated cells to fuse together to form multi-nucleated cells. Accordingly, based on the fusion index or fusion distribution determined for each test composition as described above, the analysis module can provide a ranking of the anabolic efficacy of the test compositions to stimulate muscle cell proliferation or differentiation. The analysis module can also be configured to rank the test compositions based on a quantifier as determined by other methods for quantifying muscle growth as described herein.

In the analysis of bone cell proliferation and/or differentiation, the analysis module can provide a ranking of the test compositions with respect to their individual abilities to stimulate bone cell proliferation and/or differentiation. The analysis module can be configured to rank the test compositions based on bone growth as determined by various methods for quantifying bone growth as described herein. In one embodiment, the ranking of the test compositions can be determined based on expression of at least one bone marker (e.g., ALP) in the cells upon the contact of the cells with the test compositions.

While the analysis module can be configured to output two separate anabolic profiles or anabolic ranking of the test compositions (e.g., one based on the test composition's ability to induce muscle proliferation and/or differentiation, and another based on the test composition's ability to induce bone proliferation and/or differentiation), in some embodiments, the analysis module can be further configured to generate a combined anabolic profile or anabolic ranking of the test compositions based on the two separate anabolic profiles or anabolic rankings of the test compositions. For example, in some embodiments, the analysis module can be configured to compute a weighted average of the muscle growth and bone growth for generating a combined anabolic profile or anabolic ranking of the test compositions.

In some embodiments, by providing in computer-readable form information about anabolic responses of cells to a plurality of test compositions, one can use the information in the readable form in the analysis module 80 and compare with the reference data within the storage device 30. In some embodiments, search programs can be used to perform comparison of the determined anabolic profile with the reference data. The comparison made in computer-readable form provides a computer readable comparison result which can be processed by a variety of means. Content 140 based on the comparison result can be retrieved from the determination module 40 or the analysis module 80, or it can be displayed on a display module 110.

In one embodiment, the reference data stored in the storage device 30 to be read by the determination module 40 or the analysis module 80 includes information about anabolic responses from a control, e.g., of the same type as the subject or the population subgroup to be tested. Alternatively, the reference data includes a database, e.g., anabolic profiles of a population of various subjects (e.g., subjects having various types of a musculoskeletal disease or disorder, and apparently healthy/normal subjects), that is used to facilitate selecting or optimizing a treatment regimen for a subject suffering from a similar type of musculoskeletal disease or disorder, and/or for diagnosing the type of a musculoskeletal disease or disorder.

In one embodiment, the reference data are one or more reference polynucleotide, or polypeptide sequences. In some embodiments, the reference polynucleotide sequences can be derived from nucleotide sequences of markers or molecules specific for muscle and/or bone cells, e.g., ALP or a portion there of. In some embodiments, the reference polypeptide sequences can be derived from amino acid sequences of markers or molecules specific for muscle and/or bone cells, e.g., ALP, or a portion thereof.

In one embodiment, the reference data are electronically or digitally recorded and annotated from databases including, but not limited to GenBank (NCBI) protein and DNA databases such as genome, ESTs, SNPS, Traces, Celara, Ventor Reads, Watson reads, HGTS, and the like; Swiss Institute of Bioinformatics databases, such as ENZYME, PROSITE, SWISS-2DPAGE, Swiss-Prot and TrEMBL databases; the Melanie software package or the ExPASy WWW server, and the like; the SWISS-MODEL, Swiss-Shop and other network-based computational tools; the Comprehensive Microbial Resource database (available from The Institute of Genomic Research). The resulting information can be stored in a relational data base that may be employed to determine homologies between the reference data or genes or proteins within and among genomes.

The “analysis module” 80 can use a variety of available software programs and formats to analyze the information about anabolic responses of test cells to the test compositions determined in the determination module, e.g., to analyze the images of cells taken by the determination module and store on the storage device, and/or to compute a fusion or anabolic efficacy index, e.g., based on a pre-determined equation, for each test composition. The analysis module 80 can also use a variety of available software programs and formats to perform ranking of the anabolic efficacies of different test compositions based on the computed fusion or anabolic efficacy indices.

The analysis module 80 can also use a variety of available software programs and formats to compare anabolic profiles determined in the determination module 40 to reference data. In one embodiment, the analysis module 80 is configured to use pattern recognition techniques to compare anabolic profiles from one or more entries to one or more reference data patterns. The analysis module 80 may be configured using existing commercially-available or freely-available software for comparing patterns, and may be optimized for particular data comparisons that are conducted. The analysis module 80 provides computer readable information related to anabolic responses of test cells to one or more test compositions that can include, for example, information regarding muscle cell proliferation and/or differentiation, and/or bone cell proliferation and/or differentiation, e.g., fusion or anabolic efficacy indices described earlier, detection of the presence or absence of a marker or molecule specific for muscle and/or bone cells, determination of the concentration of the marker or molecule, or determination of an expression profile. The analysis or comparison result can be further processed by calculating ratios. For example, anabolic or expression profiles can be discerned.

The analysis module 80, or any other module described herein, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment, users can directly access data (via Hypertext links for example) residing on Internet databases using a HTML interface provided by Web browsers and Web servers. In another embodiment, users can directly access data residing on the “cloud” provided by the cloud computing service providers.

Various algorithms are available which are useful for comparing data, ranking data and/or identifying the predictive anabolic signatures for a specific musculoskeletal disease or disorder. For example, algorithms such as those identified in Odibat O, and Reddy C K. (2012) “Ranking differential hubs in gene co-expression networks.” J Bioinform Comput Biol. 10: 1240002; Nel A et al. (2012) “Nanomaterial Toxicity Testing in the 21st Century: Use of a Predictive Toxicological Approach and High-Throughput Screening.” Acc Chem Res.; Nobels I. et al. (2011) “Toxicity ranking and toxic mode of action evaluation of commonly used agricultural adjuvants on the basis of bacterial gene expression profiles.” PLoS One; 6:e24139.

In one embodiment, the analysis module 80 can further compare anabolic profiles, e.g., protein expression profiles, with reference data. Any available comparison software can be used, including but not limited to, the Ciphergen Express (CE) and Biomarker Patterns Software (BPS) package (available from Ciphergen Biosystems, Inc., Freemont, Calif.). Comparative analysis can be done with protein chip system software (e.g., The Proteinchip Suite (available from Bio-Rad Laboratories, Hercules, Calif.). Algorithms for identifying expression profiles can include the use of optimization algorithms such as the mean variance algorithm (e.g. JMP Genomics algorithm available from JMP Software Cary, N.C.).

In one embodiment, pattern comparison software is used to determine whether patterns of anabolic profiles are indicative of the presence or the absence of a musculoskeletal disease or disorder in a test sample of a subject.

In various embodiments of the system or computer system described herein, the analysis module 80 can be integrated into the determination module 40.

The analysis module 80 provides computer readable comparison result that can be processed in computer readable form by predefined criteria, or criteria defined by a user, to provide a content based in part on the analysis result that may be stored and output as requested by a user using a display module 110. The display module 110 enables display of a content 140 based in part on the analysis and/or comparison result for the user. In one embodiment, the content 140 includes a signal indicative of at least a partial ranking of the anabolic efficacy of the test compositions. In another embodiment, the content 140 includes a signal indicative of at least one test composition recommended for the subject's treatment. In some embodiments, the content 140 includes a signal indicative of no test composition recommended for the subject. Any signal or medium can be used for displaying the content, for example, a display of content 140 on a computer monitor, a printed page of content 140 from a printer, or a light or sound encoding the content 140.

In one embodiment, the content 140 based on the analysis/comparison result is displayed on a computer monitor. In one embodiment, the content 140 based on the analysis/comparison result is displayed through printable media. The display module 110 can be any suitable device configured to receive from a computer and display computer readable information to a user. Non-limiting examples include, for example, general-purpose computers such as those based on any type of processors or microprocessors, e.g., INTEL® microprocessors, visual display devices such as flat panel displays, cathode ray tubes and the like, as well as computer printers of various types.

In one embodiment, a World Wide Web browser is used for providing a user interface for display of the content 140 based on the analysis/comparison result. It should be understood that other modules of the invention can be adapted to have a web browser interface. Through the Web browser, a user may construct requests for retrieving data from the analysis module. Thus, the user will typically point and click to user interface elements such as buttons, pull down menus, scroll bars and the like conventionally employed in graphical user interfaces. The requests so formulated with the user's Web browser are transmitted to a Web application which formats them to produce a query that can be employed to extract the pertinent information related to the anabolic information, e.g., display of a content comprising at least a partial ranking of the anabolic efficacy of the test compositions, or display of an indication comprising at least one test composition recommended for the subject's treatment, or display of an indication of no test composition recommended for the subject, or any display of information based thereon. In one embodiment, the reference data employed during the analysis is also displayed.

In any embodiments, the analysis module can be executed by a computer implemented software as discussed earlier. In such embodiments, a result from the analysis module can be displayed on an electronic display. The result can be displayed by graphs, numbers, characters or words. In additional embodiments, the results from the analysis module can be transmitted from one location to at least one other location. For example, the analysis/comparison results can be transmitted via any electronic media, e.g., internet, fax, phone, a “cloud” system, and any combinations thereof. Using the “cloud” system, users can store and access personal files and data or perform further analysis on a remote server rather than physically carrying around a storage medium such as a DVD or thumb drive.

The system 10, and computer readable medium 200, is merely illustrative embodiments of some aspects described herein, e.g., for performing one or more embodiments of the assays and/or methods described herein, or for using one or more kits described herein, and is not intended to limit the scope of the invention. Variations of system 10, and computer readable medium 200, are possible and are intended to fall within the scope of the invention.

The modules of the machine, or used in the computer readable medium, may assume numerous configurations. For example, function may be provided on a single machine or distributed over multiple machines.

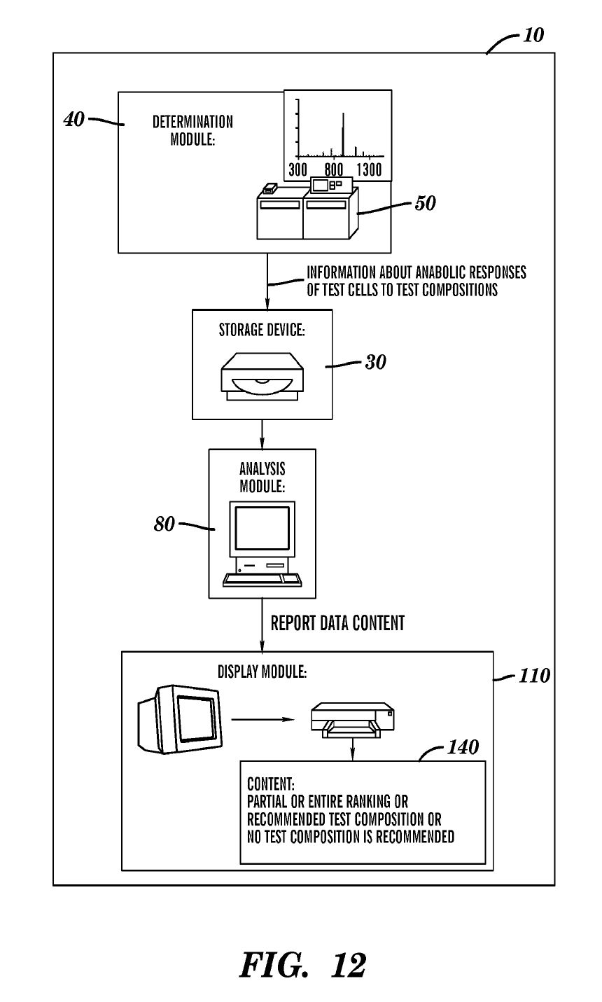

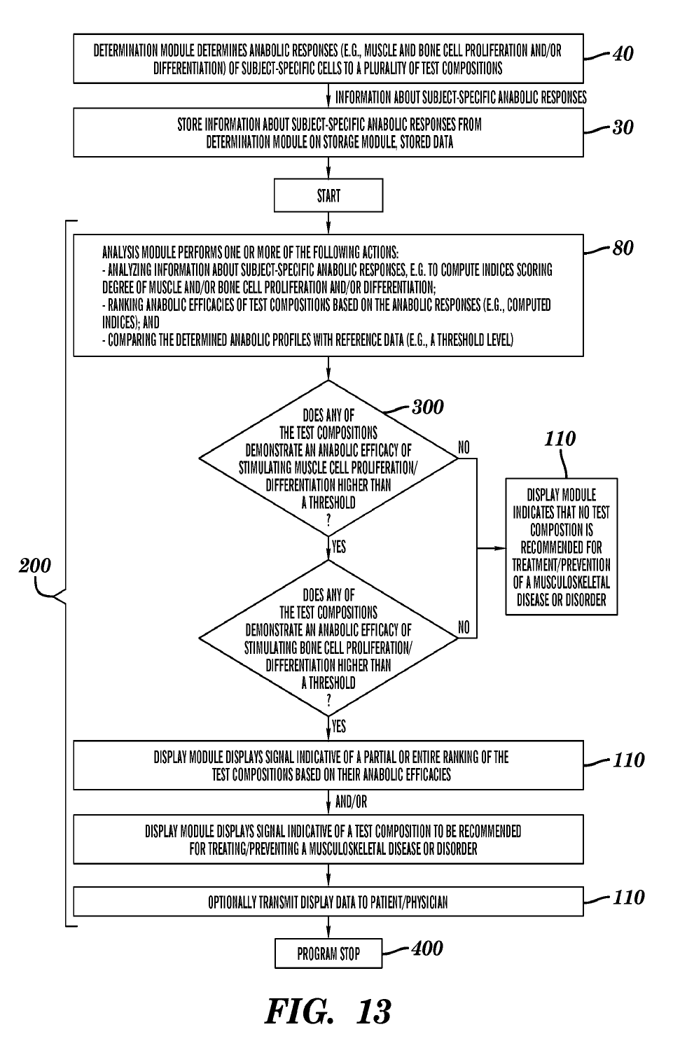

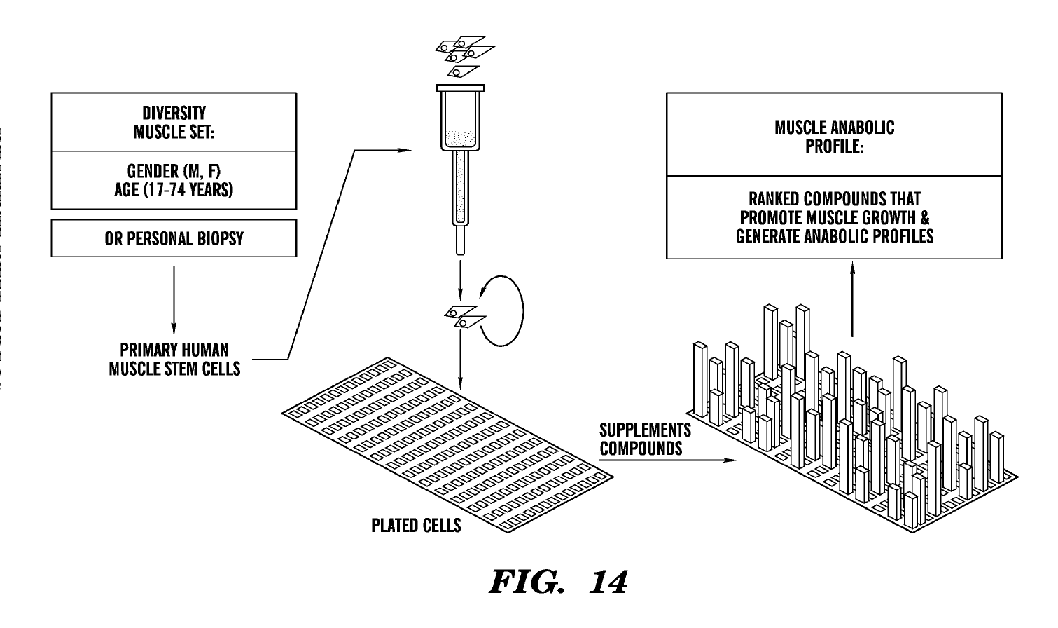

FIG. 13 is a block diagram of a computer readable media 200 according to one embodiment described herein. The system 10 shown in FIG. 12 may be a general purpose computer used alone or in connection with a specialized processing computer. Such processing may be performed by a single platform or by a distributed processing platform. In addition, such processing and functionality can be implemented in the form of special purpose hardware or in the form of software being run by a general purpose computer. Any data handled in such processing or created as a result of such processing can be stored in a temporary memory, such as in the RAM of a given computer system or subsystem. In addition, or in the alternative, such data may be stored in longer-term storage devices, for example, magnetic disks, rewritable optical disks and so on.

The system 10 (FIG. 12) may include a computer system having an operating system (e.g., UNIX) on which runs a relational database management system, a World Wide Web application, and a World Wide Web server. The software on the computer system may assume numerous configurations. For example, it may be provided on a single machine or distributed over multiple machines.

A World Wide Web browser may be used for providing a user interface. Through the Web browser, a user may construct search requests for retrieving data from a sequence database and/or a genomic database. Thus, the user will typically point and click to user interface elements such as buttons, pull down menus, scroll bars, etc. conventionally employed in graphical user interfaces. The requests so formulated with the user's Web browser are transmitted to a Web application which formats them to produce a query that can be employed to extract the pertinent information from relevant databases, e.g. reference level databases. When network employs a World Wide Web server, it supports a TCP/IP protocol. Local networks such as this are sometimes referred to as “Intranets.” An advantage of such Intranets is that they allow easy communication with public domain databases residing on the World Wide Web (e.g., the GenBank World Wide Web site). Thus, in a particular preferred embodiment of the present invention, users can directly access data (via Hypertext links for example) residing on Internet databases using a HTML interface provided by Web browsers and Web servers.

Typically the assays, methods, systems, computer-readable media and kits as disclosed herein can be used in profiling anabolic responses of musculoskeletal cells or precursor cells thereof to a plurality of test compositions. In some embodiments, the assays, methods, systems, computer-readable media and kits as disclosed herein can be used in identifying, selecting or optimizing a treatment regimen for a subject determined to have a musculoskeletal disease or disorder. In some embodiments, the assays, methods, systems, computer-readable media, and kits as disclosed herein can be used in facilitating or providing guidance for treatment and/or prevention of a musculoskeletal disease or disorder in a subject. In some embodiments, the assays, methods, systems, computer-readable media, and kits as disclosed herein can be used in screening or identifying a novel anabolic agent. In some embodiments, the assays, methods, systems, computer-readable media, and kits as disclosed herein can be used in providing guidance on a personalized treatment of a musculoskeletal disease or disorder in a subject. In some embodiments, the assays, methods, systems, computer-readable media, and kits as disclosed herein can be used to generate a personalized anabolic profile specific for a subject or patient; or stratified anabolic profiles for different population subgroups stratified by at least one or more features such as phenotypic features as described herein.

The systems and computer-readable physical medium as described above are described in the context of application on a computer system as an illustrative example, similar systems and processor-readable medium can also be developed for other processing systems or devices such as a personal digital assistant (PDA), smart-phone, cellular telephone, a tablet PC, and any other mobile devices. In some embodiments, the system does not require a determination module to quantify anabolic responses of the musculoskeletal cells or precursor cells thereof in the presence of different test compositions. Instead, in these embodiments, the system can comprise a storage device, which can be separated from or integrated into a processing system. The storage device can store a database comprising anabolic information, wherein the anabolic information for each of the population subgroups can comprise rankings of a plurality of anabolic agents based on their anabolic efficacy in each of the population subgroups. Alternatively or additionally, the system can comprise a processor-readable medium including instructions that, when executed by a processing device, can cause the processing device to access a database stored in the “cloud” system, wherein the database comprises anabolic information for each of the population subgroups stratified by at least one feature such as a phenotypic feature as described herein. Thus, based on the input of the subject's specific information comprising at least one feature such as phenotypic feature, the processor-readable medium can cause the processing device to search the database for anabolic information of an associated population subgroup characterized by the input feature (e.g., a phenotypic feature) and/or map the subject to one of a plurality of population subgroups in the database and thus output the anabolic profile of the matching or associated population subgroup and/or anabolic agents/compositions recommended for the matching or associated population subgroup.

Accordingly, provided herein is also system comprising: a computer system or processing device comprising a processor and associated memory and/or computer or processor-readable medium including instructions that, when executed by the processor, cause the computer system or processing device to perform a method comprising: (a) receiving subject-specific information comprising at least one subject's feature or phenotypic feature; (b) mapping or associating, by the processing device, a subject to one of a plurality of population subgroups in a database based on the at least one feature such as phenotypic feature, wherein the database comprises anabolic information for the plurality of the population subgroups stratified by the at least one feature such as phenotypic feature, and wherein the anabolic information for each of the population subgroups comprises rankings of a plurality of anabolic agents based on their anabolic efficacy in each of the population subgroups; and (c) displaying a content based in part on the anabolic information of the associated population subgroup, wherein the content comprises a signal indicative of at least a partial ranking of the anabolic efficacy of the anabolic agents, or a signal indicative of at least one anabolic agent recommended for the subject, or a signal indicative of no anabolic agent recommended for the subject.

In some embodiments, the mapping or associating process can comprise searching the data for anabolic information of the associated population subgroup characterized by the received feature or phenotypic feature.

The database can be stored remotely in another system over a network or stored locally in the computer system or processing device. In some embodiments where the database is stored remotely over a network, the computer system or processing device can control the operation of the remote system to transfer data between the two systems (e.g., to access data stored in the remote system and/or to save data to the remote system).

In some embodiment, the content can be displayed on a screen, a monitor, or a label, or paper. In some embodiments, the computer system or processing device can be a personal digital assistant (PDA), smart-phone, cellular telephone, a computer, a tablet PC, or any combinations thereof.

### Controls/References

A variety of appropriate controls or reference for the assays, methods, systems, kits described herein are available for use or can otherwise be generated by the skilled practitioner.

In some embodiments, a control or reference can be a negative control, which is expected to show substantially no anabolic effect (e.g., substantially no muscle or bone cell proliferation/growth or differentiation). For example, a negative control can be a group of cells that has not been administered or treated with any test composition. Alternatively, a negative control can be a group of cells treated with a composition that does not produce any anabolic effect (e.g., substantially no muscle or bone cell proliferation/growth or differentiation). Thus, a negative control can be used to account for any background signal or effect that is not contributed by the test composition. The group of cells used in a negative control can be obtained from the same test subject, a different subject or a group of subjects. In some embodiments, it is desirable to have the negative control with a similar musculoskeletal condition as the test subject.

In some embodiments, a control or reference can be a positive control, which is expected to show a significant anabolic effect (e.g., a significant increase in muscle or bone cell proliferation/growth or differentiation) and/or is used to provide a threshold level for comparison. For example, a positive control can be a group of cells treated with an agent that is known to produce a significant anabolic effect (e.g., a significant increase in muscle or bone cell proliferation/growth or differentiation). Thus, if a test composition demonstrates anabolic effects comparable to or greater than the positive control, the test composition can be considered to be an effective anabolic agent. The group of cells used in a positive control can be obtained from the same test subject, a different subject or a group of subjects. In some embodiments, it is desirable to have a positive control with a similar musculoskeletal condition as the test subject. Alternatively, a positive control can be a group of cells collected from a normal/healthy subject (e.g., a subject with no apparent symptoms for a musculoskeletal disease or disorder). These control cells (e.g., normal/healthy cells) can be administered with or without a test composition. For example, if a test composition demonstrates anabolic effects comparable to or greater than those control cells (e.g., normal/healthy cells) administered without a test composition, the test composition can be considered to be capable of restoring normal anabolic functions.

In some embodiments, a control or reference can be a collection of anabolic profiles determined from a group of subjects (e.g., subjects without any apparent symptoms for a musculoskeletal disease or disorder, and/or subjects with different kinds of musculoskeletal diseases or disorders). In these embodiments, a test subject's anabolic profiles can be compared with these control anabolic profiles, e.g., to determine or diagnose a specific musculoskeletal disease or disorder. For example, when a test subject shows similar an anabolic profile as that of particular group of control subjects having a certain musculoskeletal disease or disorder, there is a likelihood that the test subject suffers from the same musculoskeletal disease or disorder as those control subjects. However, anabolic responses (e.g., muscle cell proliferation, growth and/or differentiation, bone cell proliferation, growth and/or differentiation) can vary with various factors. Such factors may be specific to the individual (e.g. weight, age, overall health, medications or treatments undergone, prior anabolic responses, etc.). Accordingly, in some embodiments, the diagnosis can be appropriately adjusted for such factors by the skilled practitioner, when anabolic profiles or responses of a test subject are compared to outside controls (another subject or a group of subjects).

As discussed above, anabolic profiles can be determined and compared to earlier determinations in the same subject to provide useful information to the skilled practitioner in diagnosis and prognosis of the individual, regarding conditions of a musculoskeletal disease or disorder, or health conditions of a subject's muscle and/or bone. Such tracking of anabolic profiles in an individual can be useful for establishing a baseline and determining the progression of the individual with respect to muscle and bone condition, as it relates to the progressing health of the individual over the course of the various determinations of the anabolic profiles. Such determinations of anabolic profiles in a biological subject are particularly suited for tracking the progression (i.e. prognosis) or risk of a musculoskeletal disease or disorder in an individual, and also in tracking the progression or recovery of an individual following treatment or therapy. In some embodiments, such determinations of anabolic profiles in a biological subject are also particularly suited for monitoring severity of a disease in an individual (e.g., musculoskeletal deterioration) in an individual, prior to, during or following a treatment or therapy.

In one embodiment, a baseline or reference anabolic profile can be obtained from a subject at a first time point (i.e. t0) which can be prior to development of symptoms. In another embodiment, a baseline or reference anabolic profile is established after the development of symptoms (e.g. early on, midstage, in later stages) of one or more disorder, to track the progression of that disorder(s). The existence of a baseline can be useful in determining if preventative measures or existing therapies of the disease or disorder are having the desired effect in the individual. Such tracking can also indicate whether therapies or preventative measure are having a negative or no effect in the individual. Such an indication may provide the necessary feedback, to recommend other therapeutic intervention.

### Kits

A further aspect provides kits that can be used in the assays, systems, and methods of any aspects described herein. For example, in some embodiments, the kits can be used to generate a personalized diagnostic report that ranks each subject's response to the test compositions. In other embodiments, the kits can be used as diagnostic kits for optimizing or selecting an anabolic treatment of a musculoskeletal disease or disorder. In one embodiment, a kit comprises (a) a plurality of test compositions each comprising at least one agent selected to maintain and/or increase muscle and/or bone cell proliferation and/or differentiation; (b) a first container containing a first substrate material optimized for promoting muscle cell proliferation and/or differentiation; and (c) a second container containing a second substrate material optimized for promoting bone cell proliferation and/or differentiation.

The plurality of test compositions included in the kit described herein can be selected based on screening one or more libraries of compounds or small molecules (e.g., from FDA-approved compounds or NIH compounds, e.g., for any indications, but not limited to anabolic treatment, to small molecules with unknown function). See, e.g., Darcy et al., 2012 Bone. 50: 1294 for an exemplary method of a library screen to identify an anabolic agent. For example, screening one or more libraries of compounds or small molecules can identify various agents, to which subjects with distinct types of musculoskeletal disorder exhibit differential anabolic responses. When these various agents are included in the kit for profiling anabolic responses of a specific subject, not only can the subject-specific anabolic profile be used to optimize or select a personalized treatment regimen, but it can also be used to diagnose or determine a specific type of a musculoskeletal disorder based on the subject's anabolic profile. In one embodiment, the kit can comprise the plurality of test compositions as shown in Table 1 earlier.

The kit can comprise more than one test compositions, e.g., 2, 3, 4, 5, 6, 7, 8, 9, 10, 15, 20, 25, 30, 35, 40, 45, 50, 60, 70, 80, 90, 100, 150, 200, 250, 300 or more test compositions (including any value between 2 and 300). In some embodiments, the kit can comprise a range of about 30 to about 200 test compositions. In some embodiments, the kit can comprise a range of about 50 to about 150 test compositions. In some embodiments, the kit can comprise a range of about 30 to about 50 test compositions. In other embodiments, the kit can comprise at least about 40 test compositions.

Each test composition can comprise at least one agent (including 1, 2, 3, 4, 5, or more agents) selected to maintain and/or increase muscle and/or bone cell proliferation and/or differentiation. For example, in some embodiments, some test compositions can comprise more than one agents (e.g., 2, 3, 4, 5, or more agents) selected to maintain and/or increase muscle and/or bone cell proliferation and/or differentiation. In such embodiments, a combination of at least two agents (e.g., 2, 3, 4, 5, or more agents) can be included in a test composition to determine any synergistic response.

While, in some embodiments, the first substrate material and the second substrate material can be contained in a vial or a tube as a stock, in other embodiments, the first substrate material and the second substrate material can be pre-aliquoted or disposed into individual wells of a cell-culture micro-titer plate, e.g., for ease of use.

In some embodiments, each of the test compositions can be pre-distributed into individual wells of a micro-titer plate for cell culture. In some embodiments, the test compositions can be each pre-mixed into individual aliquots of the first and second substrate material.

In some embodiments, the kit can further comprise at least one micro-titer plate. In some embodiments, the kit can further comprise at least one reagent, e.g., but not limited to, cell culture medium, a cell stain (e.g., DAPI), an agent for detecting a bone marker (e.g., an antibody to a bone marker such as ALP).

In some embodiments, the kit can further comprise an agent to facilitate purification or isolation of muscle cells or precursor cells thereof from a subject's specimen (e.g., a muscle biopsy or a blood sample). For example, anti-CD45 and anti-CD46 magnetic beads can be included in the kit for use in purification or isolation of muscle cells from a muscle biopsy. In another embodiment, the kit can be used with a blood sample. Using induced pluripotent stem (iPS) cell technology, blood cell-derived muscle and bone cells are then used to generate patient specific muscle and bone cells for ex vivo therapeutics. In these embodiments, the kit can further comprise stem cell differentiation factors to generate iPS cells.

Embodiments of various aspects described herein can be defined in any of the following numbered paragraphs:
1. An assay comprising:
   (a) contacting a population of musculoskeletal cells or precursor cells thereof with a plurality of test compositions each comprising at least one agent selected to maintain or increase at least muscle growth or bone growth, to profile anabolic responses of the cells to the test compositions, wherein the musculoskeletal cells or precursor cells thereof are obtained or derived from a subject, or from a panel of cells representing at least one population subgroup;
   (b) subjecting the musculoskeletal cells or precursor cells thereof to at least two analyses to quantify muscle growth and bone growth of the cells in response to the test compositions; and
   (c) ranking anabolic efficacy of the plurality of the test compositions based on the quantified muscle growth and bone growth, thereby providing anabolic profiles for muscle and bone growth that are specific for the subject or the at least one population subgroup.
2. The assay of paragraph 1, wherein the subject is in need of anabolic augmentation or muscle loss reduction, or the subject has or has a risk for a musculoskeletal disease or disorder.
3. The assay of paragraph 1 or 2, further comprising selecting at least one of the test compositions for administration to the subject, wherein the at least one of the test compositions is selected based on the rankings of the anabolic efficacy of the plurality of the test compositions.
4. The assay of any of paragraphs 1-3, further comprising identifying or diagnosing an anabolic deficiency or anabolic resistance in the subject based on the rankings of the anabolic efficacy of the plurality of the test compositions.
5. The assay of any of paragraphs 1-4, wherein the muscle growth of at least a subset of the musculoskeletal cells or precursor cells thereof induced by each of the test compositions is quantified by an increase in the number of multi-nucleated cells formed by fusion of the musculoskeletal cells or precursor cells thereof, as compared to muscle growth in the absence of the test compositions.
6. The assay of paragraph 5, wherein at least the subset of the musculoskeletal cells or precursor cells thereof to be subjected to quantification of the muscle growth are cultured in a muscle cell-specific condition during the contact with the plurality of the test compositions.
7. The assay of paragraph 6, wherein the muscle cell-specific condition includes culturing in a first substrate material with a stiffness of about 5 kPa to about 50 kPa, or about 10 kPa to about 20 kPa.
8. The assay of any of paragraphs 1-7, wherein the bone growth of at least a subset of the musculoskeletal cells or precursor cells thereof induced by each of the test compositions is quantified by an increase in the number of bone cells differentiated from the musculoskeletal cells or precursor cells thereof, as compared to bone growth in the absence of the test compositions.
9. The assay of paragraph 8, wherein the bone cells is identified by detecting expression of a bone marker.
10. The assay of paragraph 9, wherein the bone marker includes alkaline phosphatase (ALP).
11. The assay of any of paragraphs 8-10, wherein at least the subset of the musculoskeletal cells or precursor cells thereof to be subjected to quantification of the bone growth are cultured in a bone cell-specific condition during the contact with the plurality of the test compositions.
12. The assay of paragraph 11, wherein the bone cell-specific condition includes culturing in a second substrate material with a stiffness of about 10 kPa to about 150 kPa, or about 20 kPa to about 100 kPa.
13. The assay of paragraph 12, wherein the bone cell-specific condition further includes culturing in the presence of a bone formation-inducing agent.
14. The assay of paragraph 13, wherein the bone formation-inducing agent is selected from the group consisting of bone morphogenic factor (BMP), transforming growth factor (TGF), insulin-like growth factor (IGF), basic fibroblast growth factor (bFBF), osteogenic protein (OP), and any combinations thereof.
15. The assay of any of paragraphs 1-14, wherein the musculoskeletal cells or precursor cells are obtained or derived from a muscle biopsy.
16. The assay of any of paragraphs 1-14, wherein the musculoskeletal cells or precursor cells are obtained or derived from a blood sample.
17. The assay of any of paragraphs 1-16, wherein the at least one population subgroup is stratified based on at least one phenotypic feature.
18. The assay of paragraph 17, wherein the at least one phenotypic feature is selected from the group consisting of age groups, gender, condition, ethnicity, body types, body mass index (BMI), blood types, activity levels, chronic diseases, acute diseases, genetic polymorphisms, diet, drug resistance, treatment regime, drastic/abnormal weight loss, geographical location, and any combinations thereof.
19. The assay of any of paragraphs 1-18, wherein said at least one agent selected to maintain or increase at least muscle or bone growth includes a known therapeutic, a FDA-approved drug, an over-the-counter drug or supplement, a candidate agent for anabolic treatment, or any combination thereof.
20. A method of optimizing or selecting a treatment regimen for a subject determined to have, or have a risk for, a musculoskeletal disease or disorder, the method comprising performing the assay of any of paragraphs 1-19, wherein the musculoskeletal cells or precursor cells thereof are obtained or derived from a subject determined to have, or have a risk for, a musculoskeletal disease or disorder, or from a panel of cells representing a matching population subgroup as the subject based on at least two phenotypic features, and wherein if anabolic efficacy of at least one of the test compositions is determined to be above a threshold, said at least one of the test compositions is ranked based on its ability to stimulate muscle and bone growth, and a treatment regimen comprising a test composition selected on the basis of its ranking in the assay is recommended; and wherein if none of the test compositions is determined to have anabolic efficacy above the threshold, none of the test compositions is selected or recommended for the treatment regimen.

21. A method of treating a subject determined to have, or have a risk for, a musculoskeletal disease or disorder, the method comprising, performing the assay of any of paragraphs 1-19, wherein the musculoskeletal cells or precursor cells thereof are obtained or derived from a subject determined to have, or have a risk for, a musculoskeletal disease or disorder, or from a panel of cells representing a matching population subgroup as the subject based on at least two phenotypic features, and wherein if anabolic efficacy of at least one of the test compositions is determined to be above a threshold, said at least one of the test compositions is ranked based on its ability to stimulate muscle and bone growth, and a treatment comprising a test composition selected on a basis of its ranking in the assay is recommended; and wherein if none of the test compositions is determined to have anabolic efficacy above the threshold, none of the test compositions is selected or recommended for the treatment.
22. A method of preventing a musculoskeletal disease or disorder in a subject, the method comprising performing the assay of any of paragraphs 1-19, wherein the musculoskeletal cells or precursor cells thereof are obtained or derived from a subject who is determined to have a risk for, or is at the onset of, a muscle or bone loss, or from a panel of cells representing a matching population subgroup as the subject based on at least two phenotypic features, and wherein if anabolic efficacy of at least one of the test compositions is determined to be above a threshold, said at least one of the test compositions is ranked based on its ability to reduce or delay the onset of the muscle loss or the bone loss, and a preventive treatment comprising a test composition selected on a basis of its ranking in the assay is recommended; and wherein if none of the test compositions is determined to have anabolic efficacy above the threshold, none of the test compositions is selected or recommended for the preventive treatment.
23. A method of determining an anabolic resistance in a subject comprising performing the assay of any of paragraphs 1-19, wherein the musculoskeletal cells or precursor cells thereof are obtained or derived from a subject who is in need of muscle augmentation or muscle reduction loss, or from a panel of cells representing a matching population subgroup as the subject based on at least two phenotypic features, and wherein anabolic efficacy of at least one of the test compositions determined to be below a threshold is indicative of the subject having an anabolic resistance to the at least one of the test compositions.
24. The method of any of paragraphs 20-22, further comprising administering the selected test composition to the subject.
25. The method of any of paragraphs 20-24, wherein the threshold is anabolic response of the musculoskeletal cells or precursor cells thereof in the absence of the test compositions.
26. The method of any of paragraphs 20-24, wherein the threshold is anabolic response of musculoskeletal cells or precursor cells thereof that are obtained or derived from one or more normal healthy subjects.
27. A method of treating a musculoskeletal disease or disorder in a subject, the method comprising administering an effective amount of a test composition to the subject determined to have, or have a risk for a musculoskeletal disease or disorder, wherein the test composition was selected based upon its ranking in the assay of any of paragraphs 1-19.
28. A method of maintaining or improving muscle and/or bone health in a subject, the method comprising administering an effective amount of a test composition to the subject in need of anabolic augmentation or muscle loss reduction, wherein the test composition was selected based upon its ranking in the assay of any of paragraphs 1-19.
29. The method of any of paragraphs 20-28, wherein the musculoskeletal disease or disorder is selected from the group consisting of muscle wasting associated with HIV infection, muscle wasting associated with an eating disorder, muscle wasting associated with a metabolic disorder, muscle wasting diagnosed in cancer survivors, cachexia, muscular dystrophy, osteopenia, osteoporosis, sarcopenia, an age-related musculoskeletal disease or disorder, a musculoskeletal disease or disorder associated with anabolic resistance, and any combinations thereof.
30. A system for generating anabolic profiles for one or more subjects, the system comprising:
   (a) a computer system comprising a processor and associated memory including instructions that, when executed by the processor, cause the processor to control operation of a determination module to perform at least one analysis on one or more samples to quantify muscle growth or bone growth of cells;
   (b) the determination module configured to receive the one or more samples each comprising a population of musculoskeletal cells or precursor cells thereof, wherein the musculoskeletal cells or precursor cells thereof are in contact with a plurality of test compositions each comprising at least one agent selected to maintain or increase at least muscle growth or bone growth, and is further configured to subject the musculoskeletal cells or precursor cells thereof to at least two analyses to quantify muscle growth and bone growth of the cells in response to the test compositions;
   (c) a storage device configured to store data output from said determination module;
   (d) an analysis module configured to rank anabolic efficacy of the test compositions as a function of the data output from said determination module; and
   (e) a display module for displaying a content based in part on the data output from said analysis module, wherein the content comprises a signal selected from the group consisting of a signal indicative of at least a partial ranking of the anabolic efficacy of the test compositions, a signal indicative of at least one test composition recommended for the subject's treatment, a signal indicative of no test composition recommended for the subject, and any combination thereof.
31. The system of paragraph 30, wherein the musculoskeletal cells or precursor cells thereof in each of the samples are obtained or derived from a subject, or from a panel of cells representing a population subgroup;
32. The system of paragraph 30 or 31, wherein said an analysis module further comprises a comparison algorithm adapted to compare said data output from said determination module with reference data stored on said storage device.
33. The system of any of paragraphs 30-32, wherein the determination module is configured to determine the number of multi-nucleated cells formed by fusion of the musculoskeletal cells or precursor cells thereof for quantifying the muscle growth.
34. The system of any of paragraphs 30-33, wherein the determination module is configured to determine the number of bone cells differentiated from the musculoskeletal cells or precursor cells thereof for quantifying the bone growth.
35. The system of paragraph 34, wherein the determination module is configured to identify the bone cells based on detecting expression of a bone marker.

36. The system of paragraph 35, wherein the bone marker includes alkaline phosphatase (ALP).
37. The system of any of paragraphs 30-36, further comprising a microscope and an imaging system.
38. The system of any of paragraphs 30-37, wherein the determination module is further configured to contact the musculoskeletal cells or precursor cells thereof with the plurality of test compositions.
39. A computer readable storage medium having computer readable instructions recorded thereon to define software modules for implementing a method on a computer, said computer readable storage medium comprising:
   (a) instructions for analyzing the data stored on a storage device that in part comprises data indicative of anabolic responses of musculoskeletal cells or precursor cells thereof to a plurality of test compositions comprising at least one agent selected to maintain or increase at least muscle or bone growth; wherein the analysis ranks anabolic efficacy of the test compositions based on the data stored on the storage device;
   (b) instructions for displaying a content based in part on the data stored on the storage device, wherein the content comprises a signal indicative of at least a partial ranking of the anabolic efficacy of the test compositions, or a signal indicative of at least one test composition recommended for the subject's treatment, or a signal indicative of no test composition recommended for the subject.
40. A method of selecting an anabolic agent for a subject in need of anabolic augmentation or migitation of muscle loss or bone loss comprising:
   providing a computer system, the computer system including a processor and associated memory, a user input component and an output component;
   connecting the computer system to a database, the database comprising anabolic information for a plurality of population subgroups characterized by at least one phenotypic feature, wherein the anabolic information for each of the population subgroups comprises rankings of a plurality of anabolic agents based on their anabolic efficacy in each of the associated population subgroups;
   inputting into the computer system at least one phenotypic feature associated with a subject in need of anabolic augmentation or mitigation of muscle loss or bone loss;
   searching the database for anabolic information of an associated population subgroup characterized by the input phenotypic feature; and
   selecting at least one anabolic agent for the subject based on the ranking of the anabolic agents in the associated population subgroup.
41. A method of treating a subject who is in need of anabolic augmentation or mitigation of muscle loss or bone loss comprising:
administering at least one selected anabolic agent to a subject who is in need of anabolic augmentation or mitigation of muscle loss or bone loss, wherein the at least one selected anabolic agent is determined based on a process comprising:
   (a) providing a computer system, the computer system including a processor and associated memory, a user input component and an output component;
   (b) connecting the computer system to a database, the database comprising anabolic information for a plurality of population subgroups characterized by at least one phenotypic feature, wherein the anabolic information for each of the population subgroups comprises rankings of a plurality of anabolic agents based on their anabolic efficacy in each of the associated population subgroups;
   (c) inputting into the computer system at least one phenotypic feature associated with a subject in need of anabolic augmentation or mitigation of muscle loss or bone loss;
   (d) searching the database for anabolic information of an associated population subgroup characterized by the input phenotypic feature; and
   (e) selecting at least one anabolic agent for the subject based on the ranking of the anabolic agents in the associated population subgroup.
42. The method of paragraph 40 or 41, wherein the phenotypic features comprise age, gender, condition, ethnicity, body types, body mass index (BMI), blood types, activity levels, chronic diseases, acute diseases, genetic polymorphisms, diet, drug resistance, treatment regime, drastic/abnormal weight loss, geographical location, or any combinations thereof.
43. The method of any of paragraphs 40-42, wherein the anabolic agents are selected from the group consisting of FDA-approved drugs, over-the-counter drugs, anabolic supplements and any combinations thereof.
44. The method of any of paragraphs 40-43, wherein the anabolic efficacy of the anabolic agents is determined based on the effect of the anabolic agents on fusion of muscle precursor cells to form multi-nucleated cells.
45. The method of any of paragraphs 40-44, wherein the anabolic efficacy of the anabolic agents is determined based on the effect of the anabolic agents on differentiation of the muscle cells or bone precursor cells to bone cells.
46. The method of any of paragraphs 40-45, wherein the database is created by
   (a) for each of the plurality of the population subgroups, quantifying muscle growth and bone growth of the musculoskeletal cells or precursor cells obtained or derived from the population subgroup, upon contacting the musculoskeletal cells or precursor cells with the plurality of the anabolic agents; and
   (b) ranking anabolic efficacy of the plurality of the anabolic agents based on the quantified muscle growth and bone growth for each of the plurality of the population subgroups.
47. A system comprising: a computer system comprising a processor and associated memory including instructions that, when executed by the processor, cause the processor to perform a method comprising:
   (a) receiving subject-specific information of at least one phenotypic feature;
   (b) searching a database for anabolic information of an associated population subgroup characterized by the received phenotype feature, wherein the database comprises anabolic information for a plurality of population subgroups stratified by the at least one phenotypic feature, wherein the anabolic information for each of the population subgroups comprises rankings of a plurality of anabolic agents based on their anabolic efficacy in each of the population subgroups; and
   (c) displaying a content that comprises a signal indicative of the anabolic information of the population subgroup associated with the at least one phenotypic feature of the subject, wherein the signal is selected from the group consisting of a signal indicative of at at least a partial ranking of the anabolic efficacy of the test compositions, a signal indicative of at least one test composition recommended for the subject's treatment, a signal indicative of no test composition recommended for the subject, and any combination thereof.
48. The system of paragraph 47, wherein the database is stored remotely over a network.
49. The system of paragraph 48, wherein the instructions, when executed by the processor, further cause the processor to control operation of another system to search the database.
50. The system of paragraph 47, wherein the database is stored in the computer system.
51. The system of any of paragraphs 47-49, wherein the computer system is a personal digital assistant (PDA), smart-phone, cellular telephone, a computer, a tablet PC, and any combinations thereof
52. The system of any of paragraphs 47-51, wherein the content is displayed on a screen, a monitor, or paper.

### SOME SELECTED DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

In one respect, the present invention relates to the herein described compositions, methods, and respective component(s) thereof, as essential to the invention, yet open to the inclusion of unspecified elements, essential or not (“comprising”). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

As used herein, the term “administer” refers to the placement of a composition into a subject by a method or route which results in at least partial localization of the composition at a desired site such that desired effect is produced. Routes of administration suitable for the methods of the invention include both local and systemic administration. Generally, local administration results in a higher amount of a selected test composition and/or anabolic agent being delivered to a specific location as compared to the entire body of the subject, whereas, systemic administration results in delivery of a selected test composition and/or anabolic agent to essentially the entire body of the subject. In some embodiments, the compositions described herein are administered to subjects with a musculoskeletal disease or disorder orally. In other embodiments, the compositions described herein can be administered to subjects with a musculoskeletal disease or disorder by injection.

### Examples

The examples presented herein, in part, relate to one or more embodiments of an assay described herein to profile anabolic responses of subject-specific cells (e.g., patient-specific cells) to one or more test compositions. Selection of a test composition to be recommended for treatment and/or prevention of a musculoskeletal disease or disorder is, in part, based on the anabolic ranking of the test compositions in the assay. The examples presented herein also relate to methods to identify novel anabolic agents for treatment/prevention of a musculoskeletal disease or disorder. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the paragraphs to the invention, but are rather intended to be exemplary of certain embodiments. Any variations in the exemplified methods which occur to the skilled artisan are intended to fall within the scope of the present invention.

### Example 1

### An Exemplary Muscle Cell Screen to Identify and Rank Anabolic Compounds for their Efficiency in Stimulating Muscle Growth

There is a need for profiling muscle and bone response to known and novel anabolic compounds. First, human genetic variation and life history influence, often unpredictably, the response to therapeutic intervention. For example, HIV progression and response to drugs can be influenced by genetic polymorphisms (A. Telenti et al., 2008 Annu Rev. Pharmacol. Toxicol. 48: 227). Cancer cachexia can vary in severity and response (Tan B. H. et al. 2011 J. Genet 90: 165). Muscular dystrophy can vary in progression and responsiveness (Pegoraro E. et al., 2010 Neurology 76: 219). Second, although anabolic compounds promote gains in muscle, they do so with varying efficacy that depends on many factors, including age (See, e.g., Banerjee C. et al., 2011 Immun. Aging 8: 5). Accordingly, this Example illustrates an exemplary method to perform a muscle cell screen, which can be used to provide a patient-specific anabolic profile for making therapeutic decisions based on relative anabolic efficiency.

A total of about 5405 compounds were evaluated in about 14 plates for their efficacy in promoting muscle growth. FIG. 1 is a schematic of an exemplary methodological approach for evaluating a panel of human muscle cells from a number of donors (e.g., eight donors) for their muscle anabolic profile. Human muscle cells can be isolated or purified, e.g., from a biopsy sample or a blood sample, with magnetic beads for CD45-CD56+ cells and plated in a 96 well plate, in duplicate, containing an extracellular matrix of defined stiffness optimal to muscle growth.

In order to measure muscle-growth response of a cell to a certain test composition, the cell can be cultured in any condition that is appropriate for muscle growth. For example, in some embodiments, the cell can be cultured in an extracellular matrix (ECM) scaffold that is of a defined stiffness optimal to promote muscle differentiation, e.g., between about 5 kPa and about 40 kPa, or between about 10 kPa and about 20 kPa. See, e.g., Engler A. J. et al., 2006 Cell 126: 677. In one embodiment, the cell can be cultured in an ECM scaffold with a stiffness between about 10 kPa and about 20 kPa. The cells are usually all mononucleated (e.g., one nucleus per cell as shown in FIG. 1) before treatment with any compound. Each well of the plate can be seeded with any number of cells. For example, in some embodiments, about 1000 cells can be used in each well. Next, the plated cells in the appropriate extracellular matrix are treated with an array of anabolic compounds (including known and candidate compounds). After incubation of the cells with the compound for a period of time (e.g., at least about 24 hours, or at least about 48 hours), plates containing the treated cells can be subjected to an analysis for muscle growth and/or differentiation. For example, the cells can be stained with DAPI and imaged with a microscope (e.g., CYNTELLECT CELIGO). The images are then analyzed for distribution of nuclei per cell (see, e.g., FIG. 1) to quantify fusion index and/or fusion distribution as described above, and the efficacy of the evaluated compounds for stimulating muscle growth can ranked as a function of the frequency of cells having 2 or more nuclei per cell. In some embodiments, the efficacy of the evaluated compounds for stimulating muscle growth can be ranked as a function of the frequency of cells having 3 or more nuclei per cell.

As shown in FIG. 2A, there is a wide range in efficacy for each compound (only plate 1 is shown with 384 compounds, and the results for other plates are not shown). In some embodiments, based on the screening data, a single plate with the top 10, 20, 30, 40, 50, or more compounds, based on efficacy and representation from different classes of anabolic compounds, can be designed to provide a broad anabolic profile for each subject using the kit or assay. Muscle cells from different subjects can respond differentially to the anabolic array and each subject can have a unique anabolic profile showing a rank list of compound efficacy that is specific for their own individual cells. In some embodiments, a skilled practitioner (e.g., a clinical advisor) can provide insight into the analysis of pro-anabolic compounds influencing muscle growth and any potential contra-indications of selected compounds, based on their rank in the resultant anabolic profile.

### Example 2

### An Exemplary Bone Cell Screen to Identify and Rank Anabolic Compounds for their Efficiency in Stimulating Bone Growth

Similar to unpredictability observed in cell responses to muscle growth-inducing compounds, variation in response to pro-osteogenic compounds to treat osteopenia and osteoporosis, and more generally bone loss, can be unpredictable (Palomba S. et al., 2003 Clin. Endocrinol. 58: 365). Such anabolic responses are not well quantified, and can benefit from patient-specific profiling of pro-anabolic compounds that provide each individual utilizing the kit with a ranked list of anabolic efficacy that is specific to the subject or patient. Accordingly, this Example describes an exemplary method to perform a bone cell screen, which can be used to provide patient specific anabolic profiles, e.g., for tailoring therapy for bone loss that is specific to each patient.

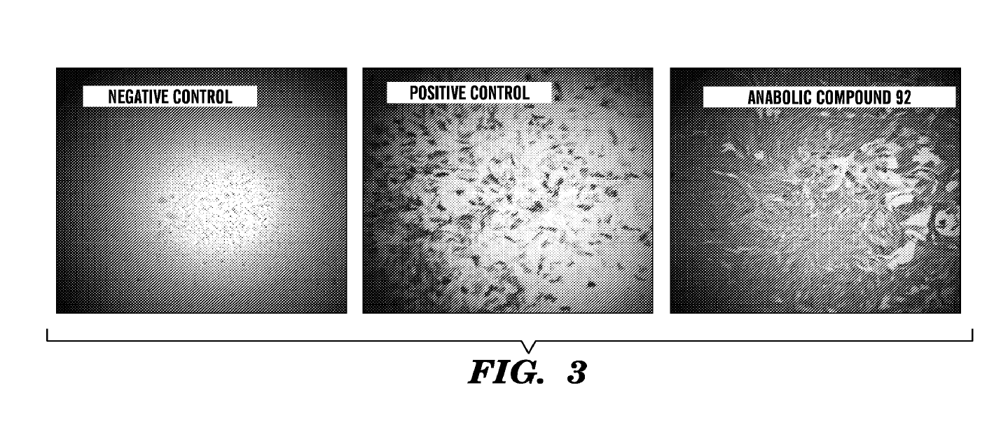

FIG. 3 shows a single result from a bone screen to identify pro-anabolic compounds stimulating bone growth. This Example employs a bone marker with essentially no background activity (negative control), but is robustly induced upon exposure to bone growth-inducing factor, e.g., bone morphogenetic protein-2 (BMP-2). While this Example illustrates the use of a specific bone marker (e.g., alkaline phosphatase (ALP)) for detection and/or quantification of bone cells, any other art-recognized bone markers can be used as well.

The influence of anabolic compounds, such as compound 92, is robust and clearly identifiable in visual screening of wells using a colorimetric assay measuring alkaline phosphatase (ALP)—a bone marker with no background in the context of the purified cell system

A panel of human cells from different donors is evaluated for their bone anabolic profile. Human muscle cells can be isolated or purified, e.g., from a biopsy sample or a blood sample, with magnetic beads for CD45-CD56+ cells and plated in a 96 well plate, in duplicate, containing an extracellular matrix of defined stiffness optimal to promoting bone growth, optionally in the presence of a bone growth-inducing factor, e.g., bone morphogenetic protein-2 (BMP-2).

In order to measure bone-growth response of a cell to a certain test composition, the cell can be cultured in any condition that is appropriate for bone growth. For example, in some embodiments, the cell can be cultured in an extracellular matrix (ECM) scaffold that is of a defined stiffness optimal to promote bone differentiation, e.g., between about 10 kPa and about 150 kPa, or between about 20 kPa and about 100 kPa. See, e.g., Engler A. J. et al., 2006 Cell 126: 677. In one embodiment, the cell can be cultured in an ECM scaffold with a stiffness between about 20 kPa and about 100 kPa. Each well of the plate can be seeded with any number of cells. For example, in some embodiments, about 1000 cells can be used in each well. Next, the plated cells in the appropriate extracellular matrix are treated with an array of anabolic compounds (including known and candidate compounds). After incubation of the cells with the compound for a period of time (e.g., at least about 24 hours, or at least about 48 hours), plates containing the treated cells can be subjected to an analysis for bone growth and/or differentiation. For example, the ALP intensity obtained from each well containing the treated cells can be determined. The date is then analyzed for ALP intensity above a threshold for BMP2 alone (e.g., a positive control) and a bone-anabolic profile specific to each subject is generated.

Bone-derived cells from different subjects can respond differentially to the anabolic array and each subject can have a unique anabolic profile showing a rank list of compound efficacy that is specific for their own individual cells. In some embodiments, a skilled practitioner (e.g., a clinical advisor) can provide insight into the analysis of pro-anabolic compounds influencing bone growth and any potential contra-indications of selected compounds, based on their rank in the resultant anabolic profile. In some embodiments, a skilled practitioner (e.g., a clinical advisor) can also interpret which compounds might be optimally useful for both muscle and bone.

### Example 3

### Demonstration of a Bone Screen in Mouse Cells

The bone screen has been demonstrated in mouse cells as described in Example 3 (Darcy et al. 2012 Bone 50:129). Novel anabolic compounds that promote bone growth were identified, in addition to known bone anabolic pro-osteogenic compounds.

Bone homeostasis can be compromised by an increase in osteoclast-mediated resorption and/or a decrease in osteoblast-mediated bone deposition. While many efforts have focused on treating osteoclast resorption, there has been less emphasis on identifying strategies for promoting osteoblast function. This Example describes a high-throughput screening assay to select for small molecules that augment bone morphogenetic protein-2 (BMP-2)-mediated osteoblast lineage commitment. After an initial screen of 5405 compounds, consisting of FDA-approved drugs, known bioactives, and compounds with novel chemical makeup, 45 small molecules that promoted osteoblast commitment were identified. Of the 45 candidates, there was a broad array of classes that included nine retinoid analogs/derivatives and four immunosuppressants, notably rapamycin and FK-506. Based on the library screen, treatment of osteoblast precursor cells thereof with rapamycin or FK-506, either alone, or synergistically with BMP-2, increased levels of phospho-Smad 1/5/8 protein and transcription of Runx-2, Osx and Smad-7, consistent with a role in promoting osteoblast differentiation. Only FK-506 was able to enhance osteocalcin transcripts and Alizarin Red staining, both late markers for differentiation. When osteoblast differentiation was suppressed with exogenous TGF-β1 treatment, rapamycin (but not FK-506) was able to rescue expression of differentiation markers, indicating distinct but overlapping activity of these compounds. Collectively, these data add to an understanding of pathways engaged in osteoblastogenesis, support a role for non-redundant immunosuppressant signaling, and provide a novel approach for the discovery of potentially therapeutic compounds that affect bone remodeling.

Loss of bone mass is an increasingly common morbidity in our aging society and is associated with an increased risk of fracture and frailty and, alarmingly, increased prevalence in chronic but treated disorders such as long-term HIV-1 infection and diabetes mellitus [1, 2]. Understanding the mechanism through which bone mass is regulated and the risk factors for bone dysregulation is a critical challenge for developing new and effective therapeutics. There are substantial data supporting crosstalk between bone forming osteoblasts and bone resorbing osteoclasts, which allow for dynamic bone remodeling necessary for bone maintenance, strength and structural integrity [3-5]. Net bone loss can occur if there is a loss in osteoblast activity or if there is an increase in osteoclast activity [6,7]. The therapeutic focus has more recently been on blocking osteoclast activity (e.g., antibody to RANKL, Denosumab [8]), and while these advances are promising, efforts to promote osteoblast activity may also be useful in establishing an arsenal of therapeutic options in countering bone loss.

High-throughput screens are becoming increasingly more common in their use to identify novel compounds as therapeutic. A clear advantage of this approach is that small molecule libraries have the capacity to probe cellular pathways to identify novel modulatory nodes with desirable effects. For example, a screen was done to identify anti-inflammatory compounds for use in cystic fibrosis and several compounds were discovered [9]. Provided herein is a novel strategy to identify osteoblast promoting compounds using a complex set of chemical libraries that range from FDA approved compounds to small molecules with unknown function.

Bone homeostasis is maintained by the proliferation and differentiation of osteoprogenitor cells, which will eventually guide the production of mineralized bone [10,11]. Osteoblast differentiation can be induced by bone morphogenetic protein (BMP)-2, initiating a signal cascade that promotes osteoblast specific genes; including the transcription factor Runx2, a critical regulator, as well as the down-stream transcription factor osterix (Osx) [12-15]. Mature osteoblasts then produce alkaline phosphatase (ALP) that can be measured via cell staining [16] and were used in this Example to identify compounds that promote osteoblastogenesis. Late differentiation is induced by another transcription factor, osteocalcin (Ocn) and this allows the cells to eventually secrete proteins that form the mineralized extra-cellular matrix [12-15].

To find novel drugs that promote osteoblast differentiation, the C2C12 cell line that tends to become muscle but can be induced to differentiate along the osteoblast lineage in the presence of BMP-2 [17] was used. The essentially no background ALP staining can help to find drugs that would enhance this efficiency and therefore potentially enhance osteoblast differentiation of pre-osteoblasts. Once the libraries were screened, 45 compounds were identified as “positive hits” that enhanced ALP expression. Two hits; FK506 and rapamycin, were selected for further evaluation because their effect on the BMP-2 pathway was unknown. Both of these immunosuppressive drugs have previously been implicated in osteoblastogenesis, however the results have been contradictory and the mechanisms of action remain unclear [18-23]. Additionally, transforming growth factor (TGF)-β1 is an anti-inflammatory cytokine that can function as both an antagonist [24-27] and agonist [28-30] on bone differentiation, depending on context. TGFβ1 was utilized under antagonistic conditions to further explore rapamycin and FK506 activity under conditions that attenuate bone.

### Results

### High-Throughput Screen Identifies Osteoblast-Inducing Compounds

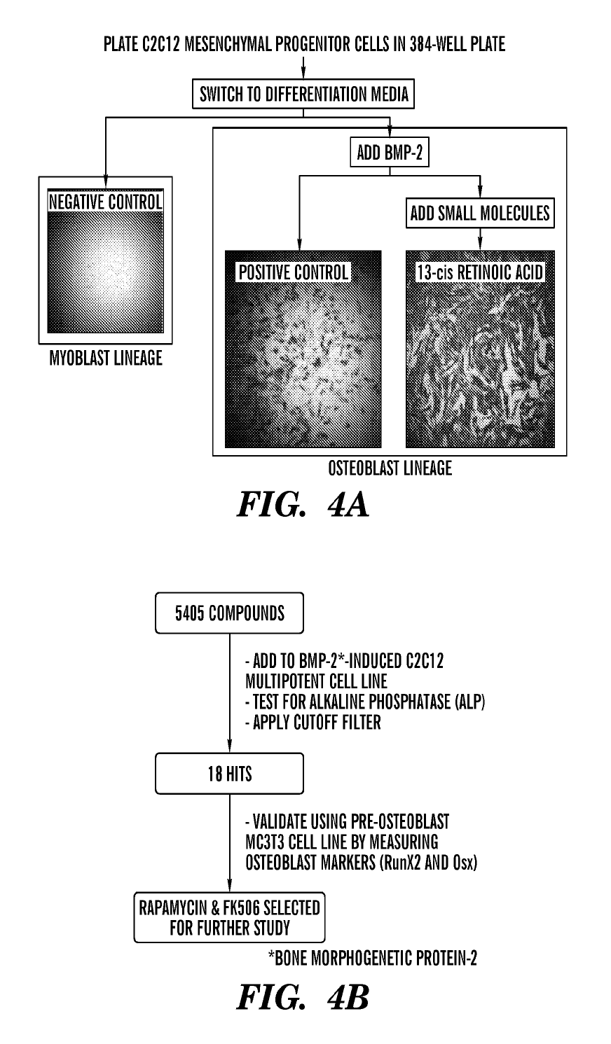

In the course of the studies on muscle differentiation using the C2C12 cell line, incomplete BMP-2-mediated conversion of C2C12 cells to osteoblasts was observed based on ALP staining with virtually no background in untreated cells. Accordingly, this system was used to screen a library of compounds that augment BMP-2 mediated osteoblast conversion. C2C12 cells were plated at 750 cells per well in a 384-square well plate and were allowed to adhere for 24 h in growth media (GM). The GM was replaced with either low serum differentiation media (DM) (negative control), DM+BMP-2 (positive control), or DM+BMP-2+ small molecules (FIG. 4A). After 72 h, cells were fixed and stained for alkaline phosphatase (ALP) and DAPI for nuclei visualization. The negative control wells expressed close to zero ALP, demonstrating the specificity of this assay. Positive control wells consistently had ALP expression in 20-30% of the cells. Initially, a pilot study of 240 FDA approved compounds was performed on this system; a number of the small molecules added in combination with BMP-2 strikingly enhanced conversion to osteoblast (ALP+) cells. Notably, there were several phenotypes observed including: no obvious effect on conversion, attenuated conversion to osteoblasts, morphological changes and enhancement or suppression of proliferation of cells. It was next sought to screen a larger set of small molecules from more diverse backgrounds to identify enhancement of osteoblast conversion.

Results from Screen of 5405 Compounds

Those compounds that enhanced osteoblast formation beyond BMP-2 alone were sought to be identified due to their therapeutic potential to promote bone formation. To ensure a robust detection of augmenters (i.e., those that promoted osteoblast differentiation), three search strategies were performed to measure the effects of added compounds' on ALP expression. After screening 5405 compounds in duplicate from five different chemical libraries, a cutoff filter was applied, which revealed 45 compounds with enhanced ALP expression. From these 45 hits (18 strongest hits and 27 potential hits, Table 2), two unexpected compounds, rapamycin and FK-506 (FK-520 was also identified but was not among the top hits), were selected to further evaluate for their effect on the pre-osteoblast mouse cell line MC3T3 (FIG. 4B). Rapamycin and FK-506 were selected to validate the screen because they are not widely recognized as osteoblast potentiators, and because their role in osteoblasts is unclear. Collectively, it was determined that screening a large number of compounds has led to discover several expected and unexpected compound hits among the libraries tested.

TABLE 2
Small compound libraries. 5 different chemical libraries were used
and the number of compounds in each library is indicated in the
table. The number of hits per library is broken down into “strongest
hits” and “potential hits.” Strongest hits were characterized
as compounds that were positive in all three analyses, whereas
potential hits were characterized as compounds that were positive
in two of the three approaches. The BU-CMLD is composed of
stereochemically and structurally complex chemical libraries
(sprioketal, epoxyquinol, oxime, macrodiolide, diketopiperazine,
cyclic ether). This library uniquely probes three-dimensional space
by employing stereochemical and positional variation within the
molecular framework as diversity elements for library design. The
NIH and FDA approved drug library, and ICCB are comprised of small
molecules that are all known bioactives. These collections were
assembled to affect a wide variety of biological pathways. The
ChemBridge represent drug-like small molecules, rationally selected
based on 3D pharmacophore analysis to cover the broadest part of
biologically relevant pharmacophore diversity space.

| Library name | Number of compounds | Strongest hits | Potential hits |
|---|---|---|---|
| NIH | 446 | 1 | 6 |
| ICCB | 480 | 13 | 13 |
| FDA | 640 | 4 | 3 |
| BUCMLD | 1839 | 0 | 1 |
| ChemBridge | 2000 | 0 | 4 |
| Total | 5405 | 18 | 27 |

Comparison and Characteristics of Results from Three Different Search Strategies (ImageJ, Digilab, Visual Inspection)

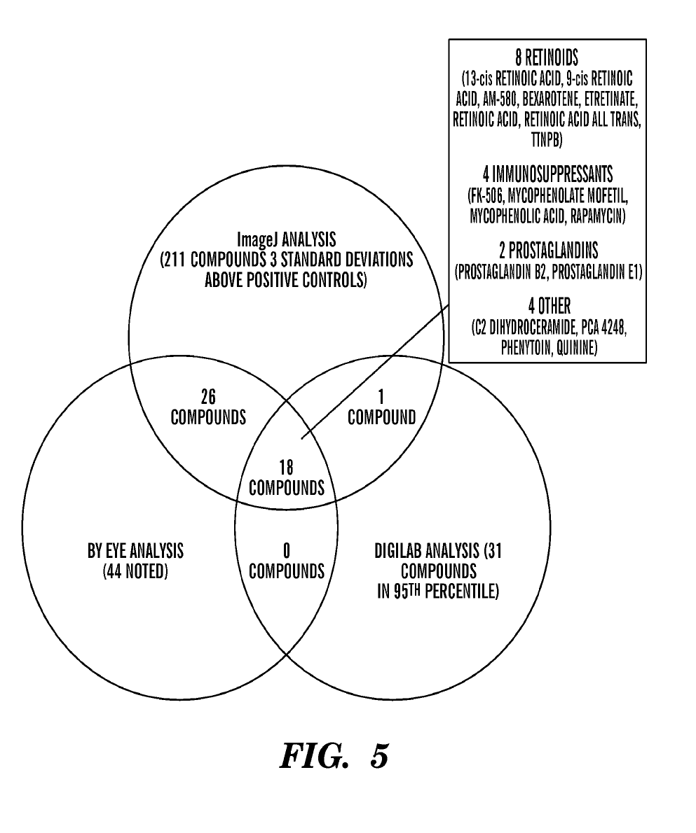

To ensure true positivity, images of wells from the initial screen of chemical libraries were processed using three different search strategies based on method of analysis (ImageJ, Digilab and visual inspection) described below to ensure reproducibility and reduce false positives. Resultant images were analyzed using ImageJ software, Digilab eaZYX Image Analyzer software, and systematically scanned, e.g., by eyes. ImageJ: analysis using ImageJ software (see Exemplary materials and methods section below) indicated 211 compounds with ALP expression greater than three standard deviations above the average (99%) of positive controls. Digilab: analysis using the eaZYX Image Analysis software from Digilab indicated 31 compounds that had ALP expression in the 95th percentile above the positive controls (see Exemplary materials and methods section below). Visual inspection: image scanning by eye revealed 44 compounds that appeared to enhance ALP expression above representative positive control wells. There were also 32 compounds that were noted by eye to have morphologic changes as compared to positive controls (data not shown). If a compound well was detected as an augmenter of ALP expression in at least two of the three analysis search strategies, the compound was considered for further analysis. Cross validation of the results from the three analyses revealed thirty-one compounds that appeared in only two out of three searches and eighteen compounds that were identified in all three searches (FIG. 5 and Table 2). Therefore, using three different methods of analysis can be used to determine the strongest positive hits.

In order to identify the functional class types of compounds represented in the top 18 hits and evaluate whether they included known and/or novel effectors, a literature search was conducted on the eighteen compounds that were identified in all three analyses and organized them into groups based on common features. Of the eighteen compounds, eight were retinoid derivatives/analogs (13-cis retinoic acid, bexarotene, TTNPB, etc.), four known immunosuppressant drugs (FK-506, mycophenolate mofetil, mycophenolic acid, and rapamycin), two prostaglandins (prostaglandin B2 and prostaglandin E1), one fatty acid (C2 dihydroceramide), one platelet-activating factor (PAF) receptor antagonist (PCA 4248), one anticonvulsant medication (phenytoin), and one antimalarial medication (quinine) (FIG. 5). There were also notable hits among the top 45 compounds including resveratrol (data not shown), which has been implicated in pro-osteogenic activity [32,33].

### Validation of Rapamycin and FK-506 Induced Osteoblastogenesis Using MC3T3 Pre-Osteoblast Cells

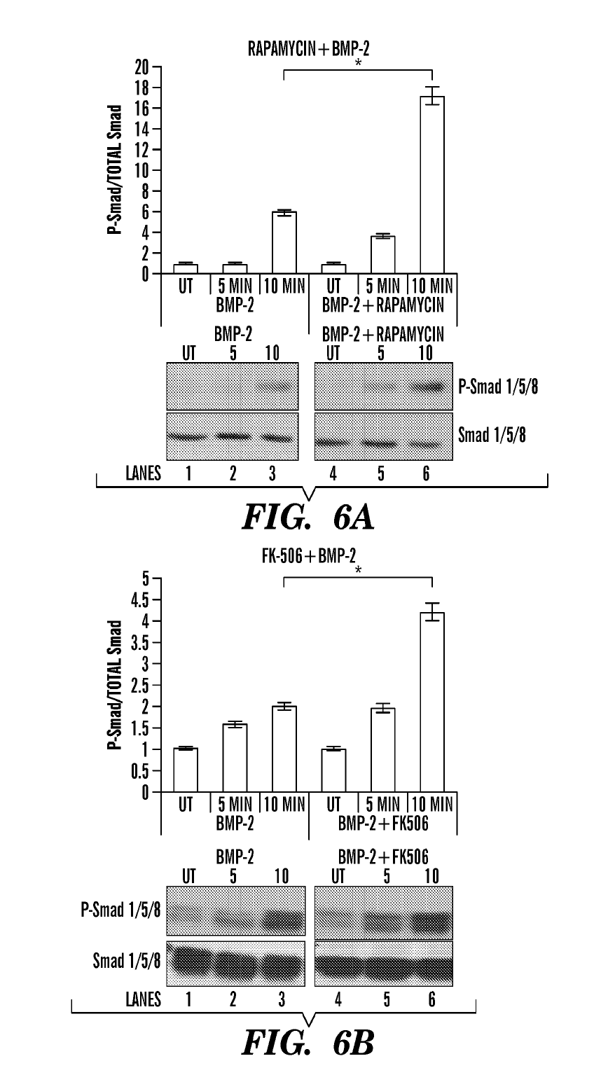

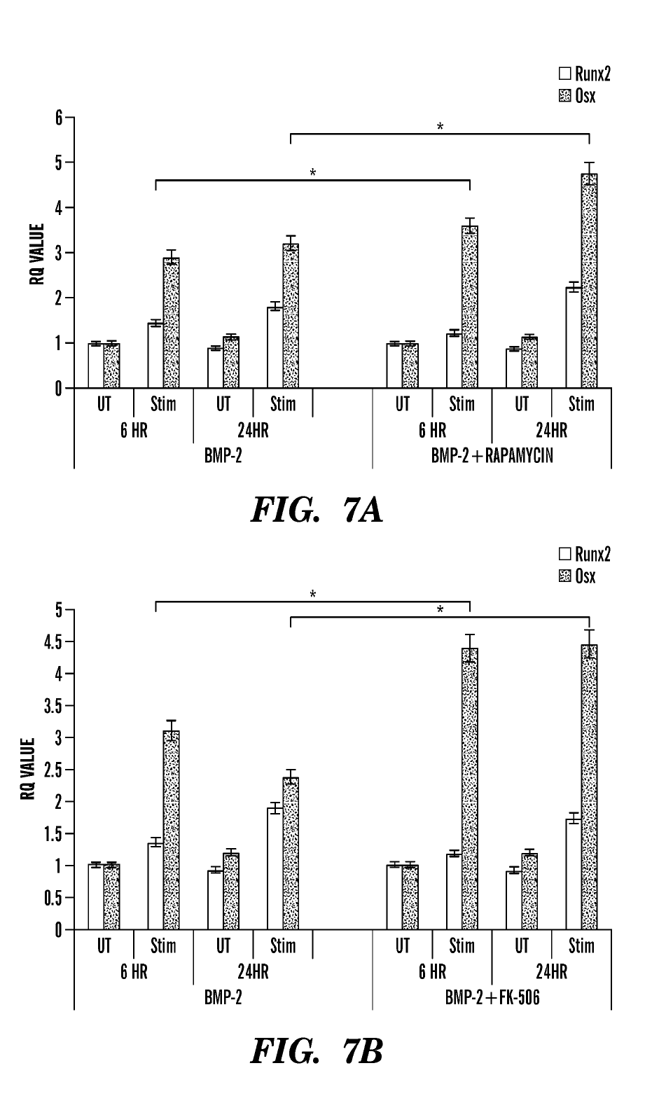

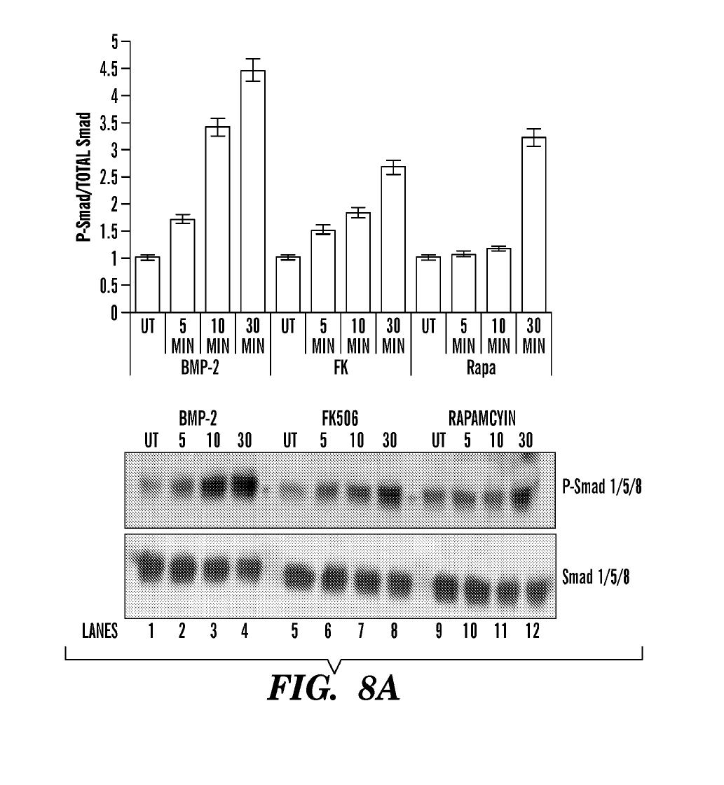

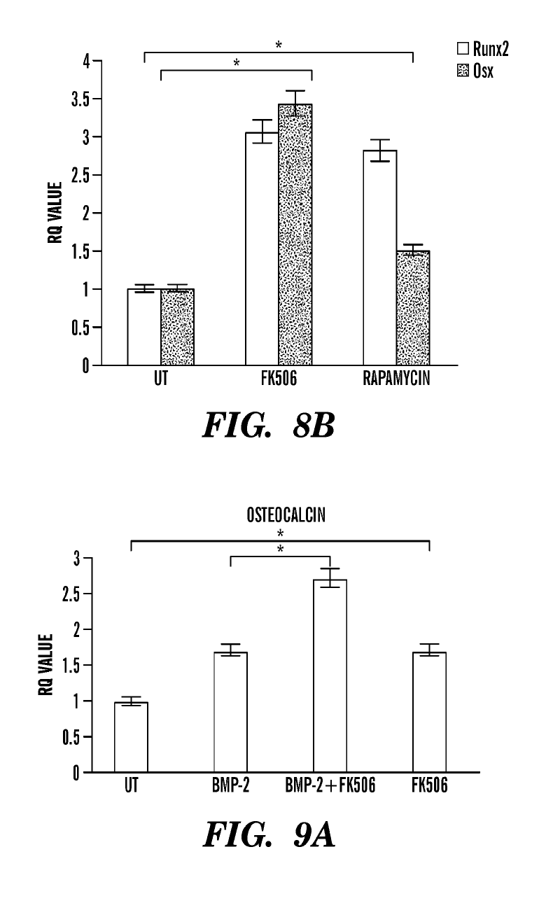

To validate the screening assay with functional assays, an osteoblast precursor cell line, MC3T3, was utilized with the two compounds rapamycin and FK506. These compounds were initially added in addition to BMP-2, which upon engagement with BMPR-I/II promotes phosphorylation of Smad 1/5/8 (P-Smad 1/5/8) and downstream activation of osteoblastogenesis such as Runx2 and Osx [4,10,14]. As shown in FIG. 6A, rapamycin was added to MC3T3-E1 cells in addition to BMP-2 and protein was collected 5 and 10 min after stimulation. Phospho-Smad 1/5/8 levels were measured via western blot and compared to total Smad 1/5/8 levels. Rapamycin significantly increased phosphorylation (lanes 3 and 6). The same experiment was carried out with FK-506 and an increase in phosphorylation was also observed (FIG. 6B, lanes 3 and 6), indicating that both immunosuppressants increase signaling of BMP-2. To determine whether or not osteoblast specific genes were increased with rapamycin and FK-506, the compounds were added to the cells with BMP-2 and then RNA was collected 6 h and 24 h after stimulation to quantify Runx-2 and Osx transcripts using quantitative real-time-PCR (qRT-PCR). Both compounds significantly increased Runx2 and Osx transcripts, although Osx had a much more robust change, especially at 24 h, both transcription factors were upregulated in reproducible experiments (FIGS. 7A-7B). Since Runx2 levels are tightly regulated and must be shut down at certain time points soon to allow for differentiation to continue, it is possible that at 6 h there is less of a change compared to 24 h. We were also interested in whether rapamycin and FK-506 require BMP-2 to augment osteoblastogenesis. To address this, bone markers in response to rapamycin and FK-506 alone was evaluated. As shown in FIGS. 8A-8B, both FK506 and rapamycin were sufficient to increase P-Smad 1/5/8 ratios (FIG. 8A, lanes 4, 8, 12) and Osx and Runx2 mRNA levels (FIG. 8B). However, the level of induction was not as dramatic as it was in the presence of exogenous BMP-2 but did reach levels similar to BMP-2 alone. Since the compounds had an effect on early differentiation capability, their influence on late differentiation was also assessed. Osteocalcin (Ocn) is an osteoblast-specific marker of late differentiation and is often observed at later time points, e.g., 14 days and later. Similar to previous experiments, FK-506 and rapamycin were added to the cells with BMP-2, with new media and compounds added every two days. Ocn transcripts were significantly increased when FK-506 was present, with or without BMP-2 (FIG. 9A), but not with rapamycin (data not shown). To assess late differentiation Alizarin-Red staining was conducted to measure mineralization. Staining was done after 21 days and images were captured demonstrating an increase in the level of mineralization with FK-506 treatment, with or without BMP-2 (FIG. 9B).

Rapamycin but not FK506 Rescues TGFβ1 Mediated Inhibition of BMP-2 Induced Runx2 and Osx and Augments Smad 7 mRNA Induction Via BMP-2

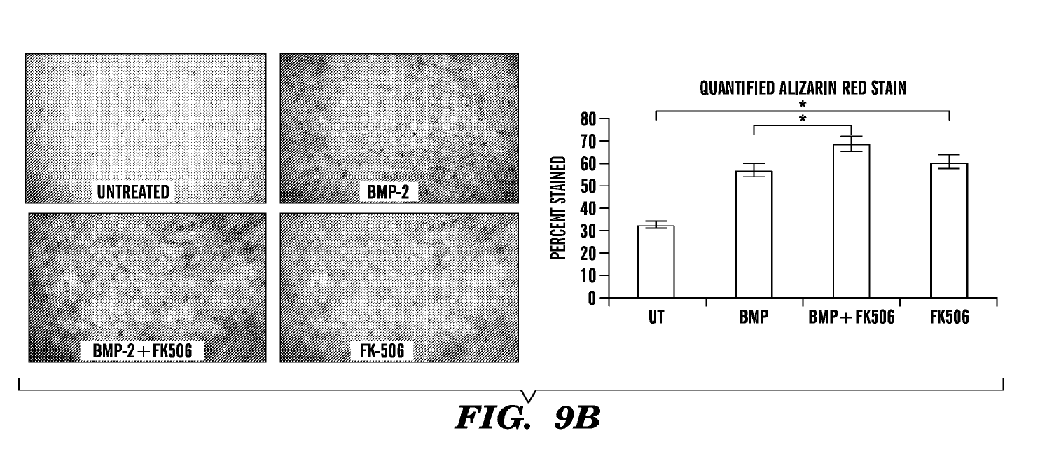

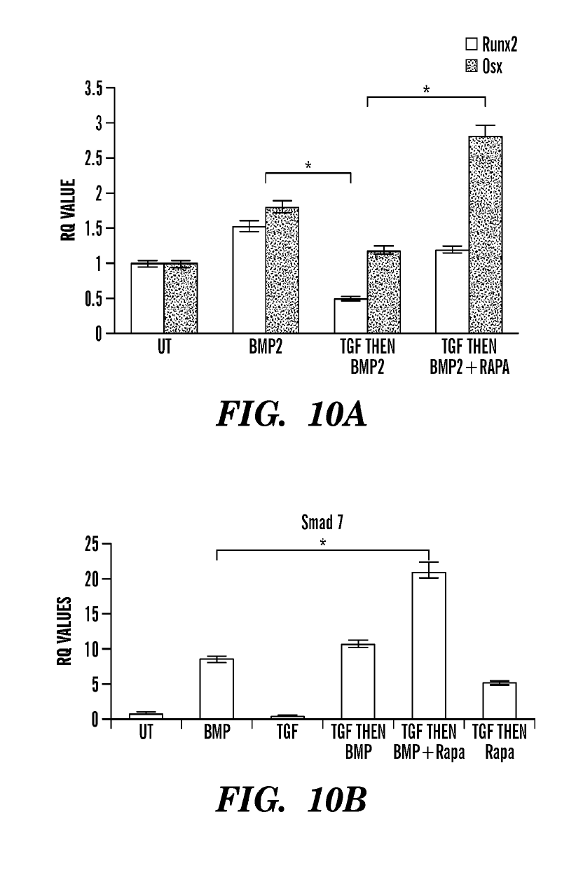

It was next sought to determine whether rapamycin and FK506 were equivalently capable of attenuating TGFβ1 mediated decline in osteoblastogenesis, an antagonistic effect that has been previously reported [7,25,34]. When TGFβ1 was added to MC3T3 cells, it reduces basal levels of Runx2 and Osx mRNA as well as preventing the induction of these transcripts by BMP-2 (FIGS. 10A-10B). However, when rapamycin was added with BMP-2 to cells after 24 h of TGFβ1 pre-treatment, the ability of TGFβ1 to inhibit osteoblast formation was attenuated (FIG. 10A). Interestingly, by contrast, when FK506 was added after TGFβ1 stimulation, there was no apparent rescue of TGFβ1 mediated suppression (data not shown). These data indicate that rapamycin interferes with the TGFβ1 consistent with these compounds having different mechanisms of action.

It was next sought to determine whether Smad-7, a suppressor of Smad-2/3 downstream of TGFβ1, might be a target of rapamycin and/or FK-506. As shown in FIG. 10B, rapamycin augmented Smad 7 expression in the context of BMP-2 and TGFβ1; whereas TGFβ1 had no apparent affect on Smad-7 levels (similar results were seen with FK-506, data not shown). Because BMP-2 alone induces Smad-7, BMP-2 signaling may involve activation of protective factors that negatively regulate Smad-2/3, further ensuring expression of bone markers, and augmented by rapamycin/FK-506. Collectively, these data indicate that while both immunosuppressants are capable of inducing bone markers, they nevertheless differ in detail.

### Discussion

Described herein is a novel high-throughput screening approach to identify bone-promoting compounds that induce osteoblastogenesis in vitro. Among over 5000 compounds from several chemical libraries that were screened, 45 compounds were identified and cross-validated using three different criteria. Two compounds, immunosuppressive drugs (i.e., rapamycin and FK506, [35]) were evaluated. The findings described herein indicate an osteogenic role for rapamycin and FK506. After validation of the use of FK506 and rapamycin in promoting osteoblastogenesis, the capacity for these compounds was evaluated to attenuate bone growth antagonists. Because TGFβ1 has a prominent role in bone loss studies and has been previously shown to inhibit osteoblastogenesis, this antagonist was selected. The findings described herein indicate a capacity for rapamycin but not FK-506 to rescue TGFβ1 mediated inhibition. By assessing these compounds in the context of antagonist perturbation that share features with common bone disease models, further insights were gained into the pathways involved in osteoblast differentiation. Notably, TGFβ1 can promote osteoblastogenesis, often in the very early stages of commitment [36]. This Example focuses on the inhibitory role due to previous reports linking increased TGFβ1 levels with decreased bone mass [37].

Among the most potent compounds identified in this screen were the retinoids, metabolically active forms of Vitamin A. Reports of in vivo experiments clearly show enhanced bone healing after injury in mice with increased Vitamin A in their diet and those studies have suggested that retinoic acid enhances osteoblast differentiation by increasing BMP2 mRNA expression[38].

Notable compounds among the strongest hits and potential hits (Table 2) included five commonly used immunosuppressant drugs (rapamycin, FK-506, FK-520, mycophenolate mofetil and mycophenolic acid) that are reported to function differently but ultimately suppress B and/or T cell proliferative responses [39-41]. Four different DNA topoisomerase inhibitors as well as two actin polymerization inhibitors were identified. Other groups that enhanced ALP expression were four prostaglandins (prostaglandins B1, E1, E2 and 13, 14-Dihydro-prostaglandin E1). It is interesting to note that the high-throughput screen described herein identified different compounds that fell into specific functional classes, indicating common mechanisms of action affecting osteoblast lineage commitment. An unexpected observation for the majority of compound hits that enhanced ALP expression in the screen was their effect on cell proliferation. A general model for an inverse relationship between proliferation and differentiation has been proposed, possibly explaining why the hits would induce differentiation at the same time they prevent proliferation[42].

It has been observed that different immunosuppressant regimens used for organ transplant have varying effects in post-transplantation bone loss [43,44]. In vitro and in vivo data show conflicting results, possibly due to the wide variation of conditions and concentrations of immunosuppressant drugs used [45]. In vitro, FK-506 has been shown to enhance osteoblast differentiation at low concentrations (10 nM-1 μM) [46]. At higher concentrations (>25 μM), FK-506 has been shown to inhibit osteoblast differentiation[47]. Interestingly, rat studies have indicated that FK-506 treatment was shown to decrease bone mineral density whereas rapamycin was shown to be bone sparing [48]. However, one study shows that FK-506 increased bone formation in alveolar bone of rats [49]. Recently, several studies have shown novel therapeutic roles for rapamycin, making it important to note that it should no longer just be thought of as an irrelevant immunosuppressive drug. One study showed that rapamycin was able to increase longevity in mice [50], whereas another recent study has shown that rapamycin can reverse the phenotype of Hutchinson-Gilford Progeria Syndrome cells [51]. For the screen described herein, the immunosuppressant drugs were tested at relatively low doses (i.e. <1 μM).

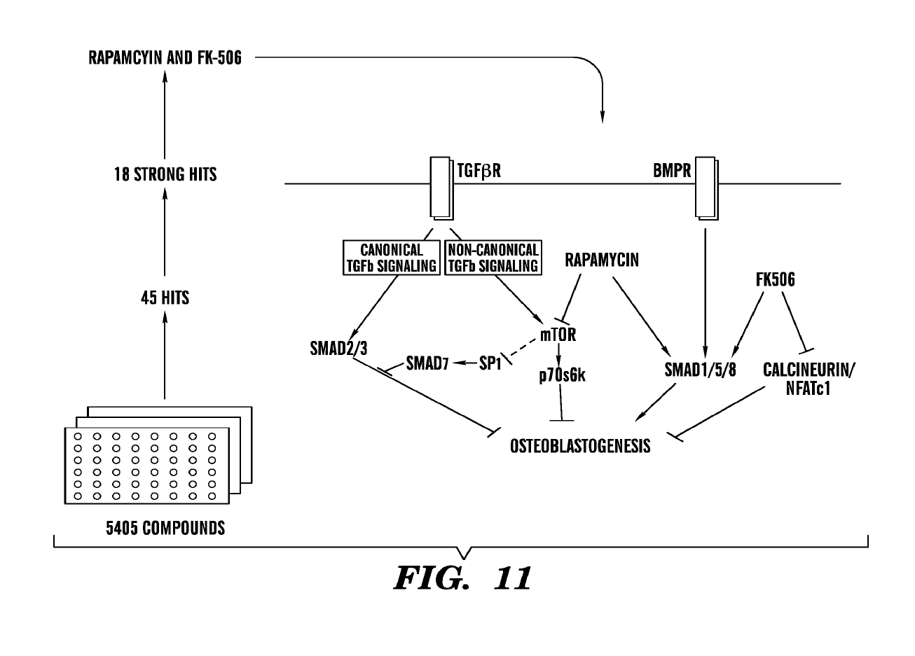

Although rapamycin and FK-506 share common signaling targets (i.e., Smad-1/5/8), they also have compound specific effects. FK-506 is a well-recognized calcineurin inhibitor and rapamycin inhibits the mammalian target of rapamycin (mTOR). Both appear to act through FKBP12 and are indicated in our graphical model (FIG. 11). Calcineurin is a phosphatase that acts upon nuclear factor of activated T cells (NFAT) allowing it to translocate into the nucleus and act as a transcription factor [52]. Constitutively active NFATc1 has been shown to inhibit osteoblast differentiation and function[53]. mTOR's role in osteoblastogenesis is not as clear but it has been shown to be critical in the differentiation of mesenchymal stem cells [18,20,54]. It is possible that rapamycin's suppression of mTOR contributes to the rescue from TGFβ1 suppression.

The capacity for both rapamycin and FK-506 to enhance osteoblastogenesis independently of BMP-2 may be due to low but detectable levels of BMP-2 present in unstimulated cells (see FIGS. 6A-6B). A model describing the augmentation of BMP-2 consistent with the findings described herein is shown in FIG. 11. In this model, both FK-506 and rapamycin promote phosphorylation of Smad 1/5/8. This, then, allows for the downstream signaling leading to activation of osteoblast specific genes. Either alone or in the presence of exogenous BMP-2, these compounds, or more potent derivatives, show promise for promoting osteoblast differentiation. Interestingly, only FK-506 was observed to enhance late differentiation, indicating that FK-506 and rapamycin signaling are non-redundant. One possible explanation may be differential effects on Runx2 levels during late differentiation, explaining why rapamycin did not enhance Ocn and mineralization. Although FK-506 also enhances Runx2, it is likely that it enhances osteoblastogenesis through a Runx2-independent mechanism as well.

TGFβ1 has been demonstrated to decrease osteoblast differentiation[27,55] and in vivo experiments have shown that blockade of TGFβ1 results in increased bone mineral density accompanied by increased osteoblast numbers [24]. Elevated TGFβ1 has been implicated in several disease states. For example, TGFβ1 is increased in the serum of HIV positive patients compared to HIV negative patients. These HIV positive patients also have increased loss of bone density compared to their HIV negative age matched controls [56,57]. Although FK-506 and rapamycin both acted to increase osteoblast formation in this evaluation system, they were not equally efficacious in attenuating TGFβ1 mediated decline in osteogenic signaling. Only rapamycin was able to attenuate the loss of differentiation. FIG. 11 shows a hypothetical model for TGFβ1 inhibition and the ability of rapamycin to rescue. Previous studies indicate that TGFβ1 can signal through two different pathways; a canonical pathway that induces Smad 2/3, which blocks osteogenesis, and a non-canonical pathway that induces mTOR, also blocking osteogenesis [58,59]. Without wishing to be bound by theory, in some embodiments, when rapamycin is introduced to pre-osteoblasts, blockage of mTOR can lead to downstream effects on the non-canonical TGFβ1 signaling pathway. The rapamycin-FKBP12 complex is known to bind mTOR and, thereby, block p70s6kinase (p70s6K) activation[60]. Rapamycin has been shown to potentiate osteoblast differentiation via a p70s6K dependent manner [61]. It has also been proposed that mTOR signaling affects Sp1 transcriptional activity [62]. Sp1 has previously been implicated in TGFβ1 signaling [63] and overexpression of Sp1 resulted in six-fold increase of basal Smad 7 promoter activity [64], indirectly enhancing Smad 7 activity, a TGFβ1 inducible antagonist [65,66]. Therefore, Smad 7 upregulation may assist but is insufficient for rapamycin to attenuate TGFβ1 induced repression of osteoblast differentiation, since FK-506 also induces Smad-7. FK-506 did not appear to significantly rescue TGFβ1 mediated decline in osteoblast differentiation.

Although this screen was focused on the identification of bone promoting compounds, an added advantage of this high-throughput approach is the utility for discovery of novel pathways with desirable outcomes. For example, the screen identified a platelet activation factor (PAF) receptor antagonist as a hit that increases osteoblast formation. Having this hit suggests the role of PAF receptors in the mechanism through which bone is formed. While there is a previous report on a potential role of PAF in bone metabolism [67], it has never been further investigated. High-throughput screens may also allow for the identification of multiple alternatives for use in patient oriented approaches by providing multiple alternative pathways that may overcome host specific deficits, such as genetic diseases in one pathway. Thus, high throughput screening is a powerful technology in translational medical research (i.e., targeted therapeutics). The more obvious benefit to high throughput screens is the ability to discover new drugs quickly and efficiently. An additional benefit is the ability to use the compound hits to explore mechanisms. For example, as shown herein, an uncommon role in promotion of osteoblast differentiation was identified for two common immunosuppressants. These two immunosuppressants and derivatives thereof can become potential therapeutics in mitigating bone loss.

### Exemplary Materials and Methods

Cell culture: C2C12. The C2C12 myoblast cell line (ATCC) was maintained in growth medium (GM) that consisted of high glucose DMEM (Gibco), 10% fetal bovine serum (Gibco), and 1% pen/strep (Invitrogen). To induce osteoblast formation, cells were allowed to reach 50% confluency, washed with PBS (1×) and switched to differentiation medium (DM) that consisted of low glucose DMEM, 2% horse serum (Gibco), 1% pen/strep and lyophilized bone morphogenetic protein-2 (BMP-2) (Genscript #Z00327), reconstituted in 20 mM acetic acid, at a concentration range of 20-200 ng/mL [17]. Negative controls were switched to DM but only received 20 mM acetic acid. Cells were passaged approximately every two days and were kept below 70% con-fluency, as per manufacturer's instructions. MC3T3-E1. MC3T3-E1 subclone 4 pre-osteoblast cells (ATCC #CRL-2593) were maintained in minimum essential medium, alpha modification (Invitrogen #A10490-01) containing 10% FBS and 1% penicillin/streptomycin. Osteoblast induction was performed by supplementing the medium with 100 ng/mL BMP-2, as previously described [31]. All cell lines used were maintained in a 37° C. incubator at 5% CO₂.

Chemical Libraries.

Chemical libraries used in this Example consisted of 640 FDA approved compounds (Enzo Life Sciences), 480 ICCB known bioactive compounds (Enzo Life Sciences), 446 compounds from the NIH Clinical Collection (BioFocus DPI), 2000 natural-like compounds, and a diversity subset of 2000 compounds (ChemBridge).

BMP-2 and Compound Stimulation.

384-square well plates (BD) were used for primary screening. PBS was added to the outermost wells to reduce edge effects. In each plate, 40 wells were used as positive controls, 28 wells were used as negative controls, and the center 240 wells were used for compound testing. All plates were screened in duplicate. The inner 308 wells received 750 cells per well and were allowed to adhere in GM for 24 h. GM was then removed, wells were washed once with PBS, and DM was added to the inner 308 wells. Negative control wells received DM. Positive control wells received BMP-2 in DM. Test wells received BMP-2 and compounds at a target concentration of 1 μM. DMSO was used as the vehicle for compound addition in this study. DMSO was also added to positive and negative control wells in equimolar amounts.

ALP and DAPI Staining.

Cells were initially fixed with a fixative solution that consisted of a 3:10:26 ratio mixture of 37% formaldehyde: citrate: acetone. Colorimetric detection of osteoblasts was achieved using the alkaline phosphatase (ALP) kit obtained from Sigma-Aldrich (Catalog #86C). Cell number was determined based on nuclei staining with DAPI nucleic acid stain as per manufacturer's instructions (Invitrogen).

Image Acquisition and Analysis.

Well images identifying ALP+ cells were acquired using a MIAS-2 plate reader, as per manufacturer's instructions (Digilab, Holliston, Mass.).

Search Strategies (ImageJ, Digilab, Visual Inspection).

Acquired images were assessed using three different search strategies (ImageJ software, Digilab software, and systematic scanning by eye); each having different sensitivities and specificities with regard to the assay.

ImageJ Analysis.

Well images were opened with the image software platform ImageJ. Images were normalized on a per plate basis and, since ALP positive cells become darker in a gray scale image, the image darkness was assessed. Images were analyzed using an Area Fraction method whereby a minimum pixel darkness threshold was applied for all image wells. All pixels that were as dark or darker than the threshold applied were converted to black. All pixels that did not meet the threshold were converted to white. The binary image was then assessed to see percentage of pixels of the image that were black (i.e. Area Fraction). Average Area Fraction from duplicate compound wells was divided by average duplicate nuclei count (as measured by DAPI count in ImageJ) to get Area Fraction on a per-cell basis. This ratio (Area Fraction: Nuclei count) was then compared to positive control wells. Ratios for the positive control wells, per plate, were determined and any compound wells that were greater than three standard deviations above the average ratio of positive control wells were considered potential augmenters of bone formation and were compared with the two other search strategies.

Digilab Analysis.

Well images were analyzed using eaZYX Image Analyzer software (Digilab, Holliston, Mass.). Cell number was determined by counting the number of DAPI stained nuclei per image. Fluorescent nuclei images were overlaid with the bright-field images to determine the number of ALP positive cells per well.

Visual Inspection Analysis.

The image of a representative positive control was compared with the images of wells that had compounds added. Wells were rated categorically on a four-point scale: (1) clear augmenter of ALP expression, (2) potential augmenter that appeared to have ALP expression above the representative positive control, (3) changes in the morphology of the cells, and (4) did not change or suppressed ALP expression. Duplicate wells that were independently confirmed for increased ALP expression were compared with the other two search strategies.

Quantitative RT-PCR.

Total RNA was extracted by the TRIzol method as recommended by the manufacturer (Invitrogen). Isolated RNA was cleaned up using the Rneasy Kit (Qiagen) and 500 ng of RNA was reverse-transcribed using the High Capacity cDNA Synthesis Kit (Applied Biosystems #4368813). Taqman expression assays were used for detection. The expression of 18S (Applied Biosystems #4319413E) was used for normalization of gene expression values. Real-time primers Runx2, Sp7 (Osx), Bglap1 (Ocn) and Smad 7 were obtained from Applied Biosystems (Mm00501580_ml, Mm00504574_ml, Mm03413826_ml and Mm00484742_ml respectively). Quantification was determined using the ΔΔCT method and normalized to the untreated sample.

Western blot. MC3T3 cells were lysed, on ice, for 20 min using lysis buffer containing 10 mM Tris, pH 7.6, 150 mM NaCl, 2 mM EDTA, 1% Triton, 0.1% SDS, 0.1 g deoxycholic acid, 1× protease inhibitor cocktail (Roche), 500 mM sodium fluoride, 100 mM sodium pyrophosphate, and 400 mM β-glycerophosphate and centrifuged at 16,000 rpm for 10 min at 4° C. Protein concentrations were determined using the BCA protein assay kit (Pierce). Equal amounts of protein (20 μg) were resolved by SDS polyacrylamide gel electrophoresis. Gels were transferred to Trans-Blot Transfer Medium Pure Nitrocellulose Membrane (Bio-Rad #162-0115) and probed with either Phospho-Smad 1/5/8 (Cell Signaling 9511S) or Smad-1/5/8 (Santa Cruz SC-6031R), as primary antibodies overnight at 4° C. in 5% milk. After washing, membranes were probed with the corresponding secondary anti-rabbit HRP-conjugated (Cell Signaling 7074S) for 1 h at room temperature. Bands were visualized by chemiluminescence using Amersham ECL Western Blotting Detection Reagents (GE Healthcare RPN2106). To observe correlating amounts of total protein and phosphoprotein, the same blot was stripped for 30 min at 65° C. in 62.5 mM Tris pH 6.8, 2% SDS and 0.6% β-mercaptoethanol. Stripped blots were washed for 30 min and then re-blocked before the primary antibody. Bands were analyzed for density with ImageJ and normalized to loading control Smad 1/5/8. Values represent fold change compared to untreated samples.

Alizarin Red Staining.

Cells were fixed with 2.5% glutaraldehyde after 21 days of stimulation and washed with PBS adjusted to a pH of 4.2. They were stained with 2% Alizarin Red S (Sigma-Aldrich A5533-25G) for 20 min at 37° C. After being washed with PBS four images were captured for each well.

### Example 4

### An Exemplary Protocol for Human Muscle Precursor Cells Thereof (MPCs) Preparation and Anabolic Screening

Below is an example protocol for human muscle precursor stem cell preparation and anabolic screening and is not construed to be limiting. Modifications to the protocol (e.g., but not limited to, cell culture medium, cell seeding density, and/or cell culture conditions) within one of skill in the art are also within the scope of the inventions described herein.
1. Transfer a ˜100-300 mg muscle micro-biopsy (e.g., collected from quadriceps or other muscles) to a container containing cell growth medium, on ice (e.g., ˜15 ml conical containing ˜10 ml DMEM+1% Penicillin/Streptomycin (P/S)).
2. Trim the biopsy sample to remove any visible fat or tendon. If none is visible, evaluate again after the digestion step later in the protocol.
3. Transfer the specimen to a container containing growth medium and digestion enzymes (e.g., a ˜50 ml conical with ˜15 ml DMEM, 1% P/S, 0.2% Collagenase type HA (Sigma)).
4. Incubate at 37° C. for about 60-90 minutes, with mild agitation.
5. Wash cells several times with a buffered solution (e.g., phosphate buffered saline (PBS)) and/or growth medium at room temperature. An example growth medium (GM) includes Ham's F10, 10% FBS, ˜5-9 ng/ml human FGFb, 1% P/S).
7. Add GM (e.g., ˜5 mL) and begin trituration. For examples, trituration can be begun with inverted, sterile glass Pasteur pipettes, to gently pull muscle apart. A Pasteur pipette can be modified by removing tip for a mid-size opening.
8. Aspirate the fragmented myofibers supernatant and transfer to a new container (e.g., ˜10 ml conical).
9. Add a buffered solution (e.g., PBS) to the plate, rinse and aspirate again—transferring supernatant to the container (e.g., ˜10 ml conical).
10. Centrifuge the container containing myofiber fragments. For example, perform the centrifugation at ˜1200 rpm on a standard tissue culture rotor for about 45-60 seconds.
11. Repeat wash with a buffered solution (e.g., PBS) and centrifuge. Use magnetic beads coated with CD45 binding molecules to remove CD45+ cells.
12. Resuspend the remaining MPCs (muscle precursor stem cells) in GM.
14. Transfer MPCs to a plate (e.g., ˜10 cm) coated with cell adhesion molecules. For example, in one embodiment, the MPCs can be seeded on a plate pre-coated with ECM and/or Matrigel. The pre-coated plates can be made with Matrigel at ˜1:100 to ˜1:250 dilution (or Sigma's Engelbreth-Holm-Swarm sarcoma ECM). Within 5-7 days observe under microscope to see activated MPCs. Within ˜6-8 days, there should be MPC outgrowth. Continue to culture the outgrowth MPC cells in GM. When passaging cells, resuspend MPCs using Ca-free PBS.
15. Use magnetic beads (e.g., coated with CD56 binding molecules) to positively select for CD56+ cells.
16. Remove fibroblasts using magnetic beads or by performing a fibroblast plate adherence depletion. An exemplary fibroblast plate adherence depletion assay includes lifting cells in PBS, pelleting by centrifugation, and replating suspended MPCs on non ECM-coated plates in GM. Fibroblast populations can attach to non-coated plates while MPCs generally do not. Thus, fibroblast cells are allowed to attach for about 10-30 minutes. The supernatant comprising the MPCs cells are then transferred to fresh ECM pre-coated plates.
16. Maintain MPCs in GM. The cells are now ready for use in the assays, methods, systems, and kits described herein.
17. To differentiate MPCs and measure muscle growth, plate about 1000 MPCs/well in a 384 well plate, each well containing a differentiation medium (DM: a low serum and 2% GM).
18. Monitor differentiation with cell imaging of nuclei distribution per cell.
19. Add test compositions, each comprising at least one anabolic agent, into the wells of the plate at a pre-determined concentration (e.g., ˜10 micromolar for each anabolic agent in DM).
20. Optionally add personalized serum from human subject blood sample. For example, 10% by volume of personalized serum can be added into DM.
21. Measure nuclei distribution of treated cells and controls after at least about 48 hours in culture.
22. Rank anabolic response based on the frequency and distribution of multi-nucleated cells, and thus provide rankings of the test compositions based on their anabolic efficacy in the muscle cells collected from a personal biopsy or from a population subgroup.

### REFERENCES

[1] Arora S, Agrawal M, Sun L, Duffoo F, Zaidi M, Iqbal J. HIV and bone loss. Curr Osteoporos Rep 2010; 8:219-26.
[2] Wongdee K, Charoenphandhu N. Osteoporosis in diabetes mellitus: possible cellular and molecular mechanisms. World J Diabetes 2011; 2:41-8.
[3] Okamoto M, Murai J, Imai Y, Ikegami D, Kamiya N, Kato S, et al. Conditional deletion of Bmpr1a in differentiated osteoclasts increases osteoblastic bone formation, increasing volume of remodeling bone in mice. J Bone Miner Res 2011; 10:2511-22.
[4] Eriksen E F. Cellular mechanisms of bone remodeling. Rev Endocr Metab Disord 2010; 11:219-27.
[5] Raggatt L J, Partridge N C. Cellular and molecular mechanisms of bone remodeling. J Biol Chem 2010; 285:25103-8.
[6] Li W, Yeo L S, Vidal C, McCorquodale T, Herrmann M, Fatkin D, et al. Decreased bone formation and osteopenia in lamin a/c-deficient mice. PLoS One 2011; 6: e19313.
[7] Rufo A, Del Fattore A, Capulli M, Carvello F, De Pasquale L, Ferrari S, et al. Mechanisms inducing low bone density in Duchenne muscular dystrophy in mice and humans. J Bone Miner Res 2011; 26:1891-903.
[8] Cummings S R, San Martin J, McClung M R, Siris E S, Eastell R, Reid I R, et al. Denosumab for prevention of fractures in postmenopausal women with osteoporosis. N Engl J Med 2009; 361:756-65.
[9] Giddings A M, Maitra R. A disease-relevant high-content screening assay to identify anti-inflammatory compounds for use in cystic fibrosis. J Biomol Screen 2010; 15:1204-10.
[10] Dallas S. Dynamics of the transition from osteoblast to osteocyte. Ann N Y Acad Sci 2010; 1192:437-43.
[11] Aubin J E. Osteoblast and chondroblast differentiation. Bone 1995; 17:77S-83S.
[12] Stein G S. Transcriptional control of osteoblast growth and differentation. Physiol Rev 1996; 76:593-618.
[13] Rosen V. BMP2 signaling in bone development and repair. Cytokine Growth Factor Rev 2009; 20:475-80.
[14] Nakashima K. The novel zinc finger-containing transcription factor osterix is required for osteoblast differentiation and bone formation. Cell 2002; 108: 17-29.
[15] Yamaguchi A. Regulation of osteoblast differentiation mediated by none morphogenetic proteins, hedgehogs, and cbfa1. Endocr Rev 2000; 21:393-411.
[16] Hoemann C D, El-Gabalawy H, McKee M D. In vitro osteogenesis assays: influence of the primary cell source on alkaline phosphatase activity and mineralization. Pathol Biol (Paris) 2009; 57:318-23.
[17] Katagiri T, Yamaguchi A, Komaki M, Abe E, Takahashi N, Ikeda T, et al. Bone morphogenetic protein-2 converts the differentiation pathway of C2C12 myoblasts into the osteoblast lineage. J Cell Biol 1994; 127:1755-66.
[18] Singha U K, Jiang Y, Yu S, Luo M, Lu Y, Zhang J, et al. Rapamycin inhibits osteoblast proliferation and differentiation in MC3T3-E1 cells and primary mouse bone marrow stromal cells. J Cell Biochem 2008; 103:434-46.
[19] Ogawa T. Osteoblastic differentiation is enhanced by rapamycin in rat osteoblast-like osteosarcoma (ROS 17/2.8) cells. Biochem Biophys Res Commun 1998; 249: 226-30.
[20] Lee K. Rapamycin promotes the osteoblastic differentiation of human embryonic stem cells by blocking the mTOR pathway and stimulating the BMP/Smad path-way. Stem Cells Dev 2010; 19:557-68.
[21] Kaihara S. Simple and effective osteoinductive gene therapy by local injection of a bone morphogenetic protein-2-expressing recombinant adenoviral vector and FK506 mixture in rats. Gene Ther 2004; 11:439-47.
[22] Kugimiya F. Mechanism of osteogenic induction by FK506 via BMP/Smad path-ways. Biochem Biophys Res Commun 2005; 338:872-9.
[23] Tang L. FK506 enhanced osteoblastic differentiation in mesenchymal cells. Cell Biol Int 2001; 26:75-84.
[24] Edwards Inhibition of TGF-beta signaling by 1D11 antibody treatment increases bone mass and quality in vivo. J Bone Miner Res; 2010.
[25] Ehnert S. TGF-b1 as possible link between loss of bone mineral density and chronic inflammation. PLoS One 2010; 5:e14073.
[26] Kang J S. Repression of Runx2 function by TGF-b through recruitment of class II histone deacetylase by Smad3. Eur Mol Biol Org 2005; 24:2543-55.
[27] Chambers T J. Regulation of the differentiation and function of osteoclasts. J Pathol 2000; 192:4-13.
[28] Centrella M. Transforming growth factor beta is a bifunctional regulator of replication and collagen synthesis in osteoblast enriched cell cultures from fetal rat bone. J Cell Biochem 1987; 262:2869-74.
[29] Centrella M. Multiple regulatory effects by transforming growth factorbeta on type I collagen levels in osteoblast-enriched cultures from fetal rat bone. Endocrinology 1992; 131:2863-72.
[30] Wrana J L. Differential effects of transforming growth factor-beta on the synthesis of extracellular matrix proteins by normal fetal rat calvarial bone cell populations. J Cell Biol 1988; 106:915-24.
[31] Takuwa Y, Ohse C, Wang E A, Wozney J M, Yamashita K. Bone morphogenetic protein-2 stimulates alkaline phosphatase activity and collagen synthesis in cultured osteoblastic cells, MC3T3-E1. Biochem Biophys Res Commun 1991; 174: 96-101.
[32] Rayalam S, Della-Fera M A, Baile C A. Synergism between resveratrol and other phytochemicals: implications for obesity and osteoporosis. Mol Nutr Food Res 2011; 55:1177-85.
[33] Uysal T, Gorgulu S, Yagci A, Karslioglu Y, Gunhan O, Sagdic D. Effect of resveratrol on bone formation in the expanded inter-premaxillary suture: early bone changes. Orthod Craniofac Res 2011; 14:80-7.
[34] Matsumoto T. TGF-b-related mechanisms of bone destruction in multiple myeloma. Bone 2011; 48:129-34.
[35] Taylor A L, Watson C J, Bradley J A. Immunosuppressive agents in solid organ transplantation: mechanisms of action and therapeutic efficacy. Crit Rev Oncol Hematol 2005; 56:23-46.
[36] Tang Y, Wu X, Lei W, Pang L, Wan C, Shi Z, et al. TGF-beta1-induced migration of bone mesenchymal stem cells couples bone resorption with formation. Nat Med 2009; 15:757-65.
[37] Maeda S, Hayashi M, Komiya S, Imamura T, Miyazono K. Endogenous TGF-beta signaling suppresses maturation of osteoblastic mesenchymal cells. EMBO J 2004; 23:552-63.
[38] Tanaka K, Tanaka S, Sakai A, Ninomiya T, Arai Y, Nakamura T. Deficiency of vitamin A delays bone healing process in association with reduced BMP2 expression after drill-hole injury in mice. Bone 47: 1006-1012.
[39] Ohdan H. Quantification of T-cell proliferation for individualizing immunosuppressive therapy for transplantation patients. Clin Pharmacol Ther 87: 23-26.
[40] Sehgal S N. Rapamune (RAPA, rapamycin, sirolimus): mechanism of action immunosuppressive effect results from blockade of signal transduction and inhibition of cell cycle progression. Clin Biochem 1998; 31:335-40.
[41] Hamawy M M. Molecular actions of calcineurin inhibitors. Drug News Perspect 2003; 16:277-82.
[42] Zhu L, Skoultchi A I. Coordinating cell proliferation and differentiation. Curr Opin Genet Dev 2001; 11:91-7.
[43] Tamler R, Epstein S. Nonsteroid immune modulators and bone disease Ann N Y Acad Sci 2006; 1068:284-96.
[44] Epstein S, Shane E, Bilezikian J P. Organ transplantation and osteoporosis. Curr Opin Rheumatol 1995; 7:255-61.
[45] Yeo H, Beck L H, McDonald J M, Zayzafoon M. Cyclosporin A elicits dose-dependent biphasic effects on osteoblast differentiation and bone formation. Bone 2007; 40: 1502-16.
[46] Tang L, Ebara S, Kawasaki S, Wakabayashi S, Nikaido T, Takaoka K. FK506 enhanced osteoblastic differentiation in mesenchymal cells. Cell Biol Int 2002; 26: 75-84.
[47] Koga T, Matsui Y, Asagiri M, Kodama T, de Crombrugghe B, Nakashima K, et al. NFAT and osterix cooperatively regulate bone formation. Nat Med 2005; 11:880-5.
[48] Romero D F, Buchinsky F J, Rucinski B, Cvetkovic M, Bryer H P, Liang X G, et al. Rapamycin: a bone sparing immunosuppressant? J Bone Miner Res 1995; 10:760-8.
[49] Andia D C, Nassar C A, Nassar P O, Guimaraes M R, Cerri P S, Spolidorio L C. Treatment with tacrolimus enhances alveolar bone formation and decreases osteoclast number in the maxillae: a histomorphometric and ultrastructural study in rats. Histol Histopathol 2008; 23:1177-84.
[50] Miller R A, Harrison D E, Astle C M, Baur J A, Boyd A R, de Cabo R, et al. Rapamycin, but not resveratrol or simvastatin, extends life span of genetically heterogeneous mice. J Gerontol A Biol Sci Med Sci 2011; 66:191-201.
[51] Cao K. Rapamycin reverses cellular phenotypes and enhances mutant protein clearance in Hutchinson-Gilford Progeria Syndrome cells. Sci Transl Med 2011; 3:1-11.
[52] Rusnak F, Mertz P. Calcineurin: form and function. Physiol Rev 2000; 80:1483-521.
[53] Choo M K, Yeo H, Zayzafoon M. NFATc1 mediates HDAC-dependent transcriptional repression of osteocalcin expression during osteoblast differentiation. Bone 2009; 45:579-89.
[54] Xiang X, Zhao J, Xu G, Li Y, Zhang W. mTOR and the differentiation of mesenchymal stem cells. Acta Biochim Biophys Sin (Shanghai) 2011; 43:501-10.
[55] Ehnert S, Baur J, Schmitt A, Neumaier M, Lucke M, Dooley S, et al. TGF-beta1 as possible link between loss of bone mineral density and chronic inflammation. PLoS One 2010; 5:e14073.
[56] Cazanave C, Dupon M, Lavignolle-Aurillac V, Barthe N, Lawson-Ayayi S, Mehsen N, et al. Reduced bone mineral density in HIV-infected patients: prevalence and associated factors. AIDS 2008; 22:395-402.
[57] Dolan S E, Kanter J R, Grinspoon S. Longitudinal analysis of bone density in human immunodeficiency virus-infected women. J Clin Endocrinol Metab 2006; 91: 2938-45.
[58] Massague J. How cells read TGF-beta signals. Nat Rev Mol Cell Biol 2000; 1: 169-78.
[59] Zhang Y E. Non-Smad pathways in TGF-beta signaling. Cell Res 2009; 19:128-39.
[60] Dumont F J, Su Q. Mechanism of action of the immunosuppressant rapamycin. Life Sci 1996; 58:373-95.
[61] Vinals F, Lopez-Rovira T, Rosa J L, Ventura F Inhibition of PI3K/p70 S6K and p38 MAPK cascades increases osteoblastic differentiation induced by BMP-2. FEBS Lett 2002; 510:99-104.
[62] Astrinidis A, Kim J, Kelly C M, Olofsson B A, Torabi B, Sorokina E M, et al. The transcription factor SP1 regulates centriole function and chromosomal stability through a functional interaction with the mammalian target of rapamycin/raptor complex. Genes Chromosomes Cancer 2010; 49:282-97.
[63] Lai C F, Feng X, Nishimura R, Teitelbaum S L, Avioli L V, Ross F P, et al. Transforming growth factor-beta up-regulates the beta 5 integrin subunit expression via Sp1 and Smad signaling. J Biol Chem 2000; 275:36400-6.
[64] Brodin G, Ahgren A, ten Dijke P, Heldin C H, Heuchel R. Efficient TGF-beta induction of the Smad7 gene requires cooperation between AP-1, Sp1, and Smad proteins on the mouse Smad7 promoter. J Biol Chem 2000; 275:29023-30.
[65] Itoh S, Landstrom M, Hermansson A, Itoh F, Heldin C H, Heldin N E, et al. Transforming growth factor beta1 induces nuclear export of inhibitory Smad7. J Biol Chem 1998; 273:29195-201.
[66] Nakao A, Afrakhte M, Moren A, Nakayama T, Christian J L, Heuchel R, et al. Identification of Smad7, a TGFbeta-inducible antagonist of TGF-beta signalling. Nature 1997; 389:631-5.
[67] Shibata Y, Ogura N, Moriya Y, Abiko Y, Izumi H, Takiguchi H. Platelet-activating factor stimulates production of prostaglandin E2 in murine osteoblast-like cell line MC3T3-E1. Life Sci 1991; 49:1103-9.

It is understood that the foregoing detailed description and examples are illustrative only and are not to be taken as limitations upon the scope of the invention. Various changes and modifications to the disclosed embodiments, which will be apparent to those of skill in the art, may be made without departing from the spirit and scope of the present invention. Further, all patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. An assay comprising:
(a) contacting a population of subject-specific musculoskeletal cells or precursor cells from a subject with a plurality of test compositions each comprising at least one agent selected to profile anabolic responses of the subject;
(b) subjecting the subject-specific cells to at least two analyses to quantify the presence or absence of anabolic response or anabolic resistance of the subject-specific cells in response to the test compositions; and
(c) ranking anabolic efficacy of the plurality of the test compositions based on the quantified anabolic response or anabolic resistance, thereby providing profiles for muscle and bone growth and/or maintenance that are personalized for the subject.

2.-30. (canceled)

31. The assay of claim 1, wherein the anabolic response or anabolic resistance of the subject-specific cells induced by the test composition is quantified based on the number of multi-nucleated cells formed by fusion of the subject-specific muscle cells, as compared to muscle growth and/or maintenance in the absence of the test composition.

32. The assay of claim 31, wherein the subject-specific cells to be subjected to the analysis for muscle anabolic response or anabolic resistance are cultured in a muscle cell-specific condition during the contact with the plurality of the test compositions.

33. The assay of claim 1, wherein the bone growth and/or maintenance of the subject-specific cells induced by the test composition is quantified based on the number of bone cells differentiated from the subject-specific cells, as compared to bone growth and/or maintenance in the absence of the test composition.

34. The assay of claim 33, wherein the bone cells is detected by expression of a bone marker.

35. The assay of claim 33, wherein the subject-specific cells to be subjected to the analysis for bone growth and/or maintenance are cultured in a bone cell-specific condition during the contact with the plurality of the test compositions.

36. The assay of claim 35, wherein the bone cell-specific condition further comprises culturing in the presence of a bone formation-inducing agent, and/or a bone maintenance agent.

37. The assay of claim 36, wherein the bone formation-inducing, and/or the bone maintenance agent comprises bone morphogenetic protein-2 (BMP-2).

38. The assay of claim 1, wherein the subject-specific cells comprise muscle cells obtained from a muscle biopsy of the subject.

39. The assay of claim 1, wherein the subject-specific cells are derived from a blood sample.

40. The assay of claim 1, wherein said at least one agent selected to induce at least muscle or bone growth, and/or prevent muscle or bone loss comprises a known therapeutic or a candidate agent for anabolic treatment.

41. A method of treating a subject determined to have a musculoskeletal disease or disorder, the method comprising, performing the assay of claim 1, wherein if anabolic efficacy of at least one of the test compositions is determined to be above a threshold, said at least one of the test compositions is ranked based on its ability to stimulate muscle and/or bone growth, or prevent muscle or bone loss, and a treatment comprising a test composition selected on a basis of its ranking in the assay is recommended; and wherein if none of the test compositions is determined to have anabolic efficacy above the threshold, none of the test compositions is selected or recommended for the treatment.

42. The method of claim 41, wherein the threshold is anabolic response of the subject-specific cells in the absence of the test composition.

43. The method of claim 41, further comprising administering an effective amount of the recommended test composition to the subject.

44. The method of claim 41, wherein the musculoskeletal disease or disorder is selected from the group consisting of muscle wasting associated with HIV infection, cachexia, muscular dystrophy, osteopenia, osteoporosis, sarcopenia, an age-related musculoskeletal disease or disorder, or a musculoskeletal disease or disorder associated with anabolic resistance.

45. A computer system for obtaining anabolic profiles from a population of musculoskeletal cells or precursor cells thereof, obtained from at least one subject, the system comprising:
(a) a determination module configured to receive muscle health data and/or a population of the musculoskeletal cells or precursor cells thereof in contact with a plurality of test compositions and to subject the cells to at least two analyses to quantify anabolic response or anabolic resistance to the test compositions;
(b) a storage device configured to store data output from said determination module;
(c) an analysis module configured to rank anabolic efficacy of the test compositions based on the data output from said determination module; and
(d) a display module for displaying a content based in part on the data output from said determination module, wherein the content comprises a signal indicative of at least a partial ranking of the anabolic efficacy of the test compositions, or a signal indicative of at least one test composition recommended for the subject's treatment, or a signal indicative of no test composition recommended for the subject.

46. A method of determining an anabolic resistance in a subject comprising performing the assay of claim 1.
